(19) United States
(12) Patent Application Publication
(10) Pub. No.: US 2026/0345574 A1
(43) Pub. Date: Nov. 5, 2026

(54) POTENT AND SELECTIVE IRREVERSIBLE INHIBITORS OF IRAK1

(71) Applicant: Dana-Farber Cancer Institute, Inc., Boston, MA (US)

(72) Inventor: John M. Hatcher, Boston, MA (US)

(21) Appl. No.: 18/880,946

(22) PCT Filed: Jul. 5, 2023
(86) PCT No.: PCT/US2023/069627

§ 371 Date: Jan. 3, 2025

Related U.S. Application Data
(60) Provisional application No. 63/367,875, filed on Jul. 7, 2022.

(51) Int. Cl. A61K 31/4545; A61K 31/4439; A61K 45/06; C07D 413/14; C07D 417/14; C07D 471/04; C07D 513/04

(52) U.S. Cl. CPC A61K 31/4545; A61K 31/4439; A61K 45/06; C07D 413/14; C07D 417/14; C07D 471/04; C07D 513/04

(57) ABSTRACT

The disclosure relates to compounds that act as irreversible inhibitors interleukin 1 (IL-1) receptor-associated kinases (IRAKs); pharmaceutical compositions comprising the compounds; and methods of treating or preventing kinase-mediated disorders, including cancer and other proliferation diseases.

### RELATED APPLICATIONS

This application claims priority to U.S. Provisional Application No. 63/367,875, filed Jul. 7, 2022, the content of which is incorporated by reference in its entirety.

### GOVERNMENT SUPPORT CLAUSE

This invention was made with government support under grant no. P50 CA100707 awarded by the National Institutes of Health. The government has certain rights in the invention.

### BACKGROUND

Interleukin 1 (IL-1) receptor-associated kinases (IRAKs) are serine/threonine kinases that play critical roles in initiating innate immune responses against foreign pathogens. Altogether there are four IRAK kinases: IRAK1 and IRAK4, which are catalytically active kinases, and IRAK2 and IRAK3, which are believed to be catalytically inactive and are hence classified as “pseudokinases” (Flannery, S., et al. Biochemical Pharmacology, 2010, 80(12), 1981-1991). IRAK1 is ubiquitously expressed with its highest expression observed in blood and immune tissues (e.g., bone marrow, lymph nodes, thymus, and peripheral blood) and hematological malignancies (Cao, Z. D., et al. Science 1996, 271(5252), 1128-1131). IRAK signaling contributes to multiple signaling pathways downstream of the Toll-interleukin receptors (TIRs) that ultimately regulate NF-κB and IFN regulatory factors (IRFs) (Rao, N., et al. Molecular and Cellular Biology 2005, 25(15), 6521-6532). In the case of NF-κB, IRAK1 mediates the downstream signals of TIRs through an interaction with MYD88 that is rapidly recruited to the receptor upon ligand binding to either IL-1R or a TLR. Subsequent phosphorylation on IRAK1 by upstream signals or through autophosphorylation is the key post-translational modification and hallmark of its activation, which allows IRAK1 to bind to TRAF6 resulting in release of the IRAK1 homodimer from MYD88 and downstream NF-κB activation (Jain, A., et al. Frontiers in Immunology 2014, 5).

The participation of IRAK1 in signaling networks of the innate immune response makes it a critical regulator of inflammation (Rinqwood, L., et al. Cytokine 2008, 42(1), 1-7), antiviral response (Wong, W., Science of Signaling 2011, 4(183), ec203), and subsequent activation of the adaptive immune response (Gottpati, S., et al., Cellular Signaling 2008, 20(2), 269-276). Consequently, an extensive investigation into physiological and pathological functions of IRAK1 in regulating these processes has been performed. In particular, these studies have implicated IRAK1 inhibition as potential treatment for myocardial contractile dysfunction following burn (Thomas, J. A., et al., American Journal of Physiology-Heart and Circulatory Physiology 2002, 283(2), H829-H836)., autoimmune conditions associated with hyper inflammation (Deng, C., et al. Journal of Immunology 2003, 170(6), 2833-2842; Jacob, C. O., Proceedings of the National Academy of Sciences of the United States of America 2,009,106(15), 6256-6261), myocardial dysfunction (Thomas J. A., et al. American Journal of Physiology—Heart and Circulatory Physiology 2003, 285(2), H597-H606), microbial septic response (Chandra, R., et al. Inflammation 2013, 36(6), 1503-1512), human myelodysplastic syndrome (MDS), and acute myeloid leukemia (AML). IRAK1 is also overexpressed and hyperphosphorylated in a subset of breast cancers: in particular, triple-negative breast cancer (TNBC).

Furthermore, in Waldenström macroglobulinemia cells, the MYD88 L265P somatic mutation is highly prevalent and responsible for malignant growth through activation of nuclear factor NF-κB. Two downstream signaling branches, one including BTK and one including IRAK1, regulate NF-κB activation in Myd88L265P expressing WM cell lines. Genetic knockdown of either BTK or IRAK1 leads to modest cell killing.

### SUMMARY

In an aspect, provided herein is a compound of Formula I:

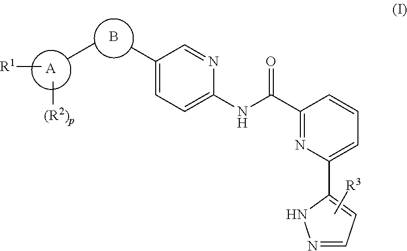

or a pharmaceutically acceptable salt thereof.

In another aspect, provided herein is a compound of Formula II:

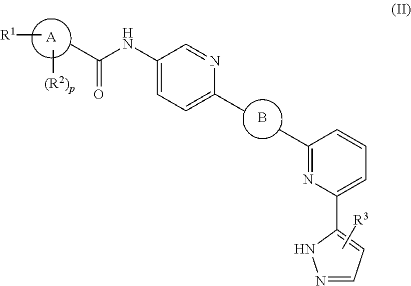

or a pharmaceutically acceptable salt thereof.

In yet another aspect, provided herein is a compound of Formula III:

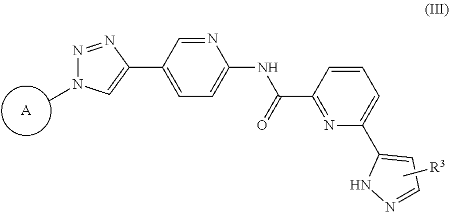

or a pharmaceutically acceptable salt thereof.

In another aspect, provided herein are pharmaceutical compositions comprising any of the compounds disclosed herein, or a pharmaceutically acceptable salt thereof, and at least one pharmaceutically acceptable carrier.

In yet another aspect, provided herein is a method of inhibiting a kinase comprising administering to a subject in need thereof an effective amount of a compound of Formula I or a pharmaceutical composition comprising a compound of the present disclosure and a pharmaceutically acceptable carrier.

In an aspect, provided herein is a method of treating a proliferation disease, comprising administering to a subject in need thereof an effective amount of a compound disclosed herein or a pharmaceutical composition comprising a compound disclosed herein and a pharmaceutically acceptable carrier.

In another aspect, provided herein is a method of treating cancer, comprising administering to a subject in need thereof an effective amount of a compound disclosed herein or a pharmaceutical composition comprising a compound disclosed herein and a pharmaceutically acceptable carrier.

The present disclosure also provides a kit comprising a compound capable of inhibiting a kinase selected from a compound disclosed herein, or a pharmaceutically acceptable salt thereof, and instructions for use in treating a proliferative disease.

### DETAILED DESCRIPTION

IRAK1 is a serine/threonine kinase that was originally identified in 1994. It is ubiquitously expressed with its highest expression observed in blood and immune tissues (for example, bone marrow, lymph nodes, thymus and peripheral blood) and hematological malignancies. IRAK signaling contributes to multiple signaling pathways downstream of the Toll-interleukin receptors (TIRs) that ultimately regulate NF-κB and IFN regulatory factors (IRFs). In the case of NF-κB, IRAK1 mediates the downstream signals of TIRs through an interaction with MYD88, which is rapidly recruited to the receptor upon ligand binding to either IL-1R or a TLR. Subsequent phosphorylation on IRAK1 by upstream signals or through autophosphorylation is the key post-translational modification and hallmark of its activation, which allows IRAK1 to bind to TRAF6 resulting in release of the IRAK1 homodimer from MYD88 and downstream NF-κB activation.

The participation of IRAK1 in signaling networks of the innate immune response has defined the enzyme as a critical regulator of inflammation, the antiviral response, and the subsequent activation of the adaptive immune response. Consequently, an extensive investigation into physiological and pathological functions of IRAK1 in regulating these processes has been performed. In particular, these studies have implicated IRAK1 inhibition as potential treatment for myocardial contractile dysfunction following burn, autoimmune conditions associated with hyper inflammation, myocardial dysfunction, microbial septic response, human myelodysplastic syndrome (MDS), and acute myeloid leukemia (AML). In Waldenström macroglobulinemia cells, the MYD88 L265P somatic mutation is highly prevalent and responsible for malignant growth through activation of nuclear factor NF-κB. Two downstream signaling branches, one including BTK and one including IRAK1, both regulate NF-κB activation in Myd88L265P expressing WM cell lines.

IRAK1 inhibitors should be pursued for the disease since genetic knockdown of either BTK or IRAK1 leads to modest cell killing; and IRAK1 is activated in viable cells isolated from WM patient currently receiving ibrutinib therapy WM cell lines, and primary patient samples treated with an IRAK1/4 inhibitor and a BTK inhibitor display augmented inhibition of NF-κB signaling and more robust cell killing. Although IRAK1 was identified over twenty years ago, and its critical function in autoimmunity and inflammation has been widely recognized, medicinal chemistry efforts directed at the development of selective inhibitors of IRAK1 have not been reported. Thus, it is important to develop selective inhibitors of IRAK (e.g., IRAK1 and IRAK4) for use as research tools as well as therapeutic agents in the treatment of diseases.

### DEFINITIONS

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, 1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with IRAK an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

An “oral dosage form” includes a unit dosage form prescribed or intended for oral administration. In an embodiment of the pharmaceutical combinations provided herein, the IRAK inhibitors disclosed herein is administered as an oral dosage form.

As used herein, the term “IRAK” refers to interleukin 1 (IL-1) receptor-associated kinases and may refer to the wild-type receptor or to a receptor containing one or more mutations.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “alkylamine” refers to the group —N(H)-alkyl or —N(alkyl)₂, wherein alkyl is defined supra.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl. As for alkyl group, haloalkyl groups can have any suitable number of carbon atoms, such as C₁₋₆.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “alkynyl” refers to a monovalent group derived from a hydrocarbon moiety containing, in certain embodiments, from two to six, or two to eight carbon atoms having at least one carbon-carbon triple bond. The alkynyl group may or may not be the point of attachment to another group. The term “alkynyl” includes, but is not limited to, ethynyl, 1-propynyl, 1-butynyl, heptynyl, octynyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, “cycloalkyl” is 3-10 membered cycloalkyl. In another embodiment, “cycloalkyl” is 3-8 membered cycloalkyl. In yet another embodiment, “cycloalkyl” is 3-6 membered cycloalkyl.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]-hexanyl, 5-azabicyclo[2.1.1]hexanyl, 6-azabicyclo[3.1.1]heptanyl, 2-azabicyclo[2.2.1]-heptanyl, 3-aza-bicyclo[3.1.1]heptanyl, 2-azabicyclo[3.1.1]heptanyl, 3-azabicyclo[3.1.0]-hexanyl, 2-azabicyclo-[3.1.0]hexanyl, 3-azabicyclo[3.2.1]octanyl, 8-azabicyclo[3.2.1]octanyl, 3-oxa-7-azabicyclo[3.3.1]-nonanyl, 3-oxa-9-azabicyclo[3.3.1]nonanyl, 2-oxa-5-azabicyclo-[2.2.1]heptanyl, 6-oxa-3-aza-bicyclo[3.1.1]heptanyl, 2-azaspiro[3.3]heptanyl, 2-oxa-6-azaspiro[3.3]heptanyl, 2-oxaspiro[3.3]-heptanyl, 2-oxaspiro[3.5]nonanyl, 3-oxaspiro[5.3]-nonanyl, 2-azaspiro[3.3]heptane, and 8-oxabicyclo[3.2.1]octanyl. In an embodiment, “heterocycloalkyl” is 3-10 membered heterocycloalkyl. In another embodiment, “heterocycloalkyl” is 3-8 membered heterocycloalkyl. In yet another embodiment, “heterocycloalkyl” is 3-6 membered heterocycloalkyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclo-penta[c]pyridinyl, 1,4,5,6-tetrahydrocyclopenta[c]pyrazolyl, 2,4,5,6-tetrahydrocyclopenta[c]-pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, “heteroaryl” is 5-10 membered heteroaryl. In another embodiment, “heteroaryl” is 5-6 membered heteroaryl.

It is to be understood that if an aryl, heteroaryl, cycloalkyl, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3-or 4-pyridinyl, the term “thienyl” means 2-or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

Provided herein are compounds that are irreversible inhibitors of interleukin 1 receptor-associated kinase (IRAK) useful in the treatment of kinase-mediated disorders, including cancer and other proliferation diseases.

In an aspect, provided herein is a compound of Formula I:

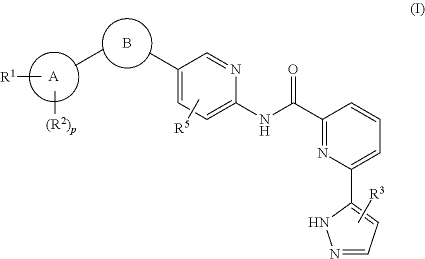

or a pharmaceutically acceptable salt thereof;
   wherein
A is 3-8 membered heterocycloalkyl;
B is oxadiazolyl;
R² is independently, at each occurrence, selected from the group consisting of halogen, C₁-C₆ alkyl, C₁-C₆ haloalkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, 5-10 membered heteroaryl, OR⁴, N(R⁴)₂, SO₂R⁴, and SR⁴;
alternatively, two R², together with the carbon atom to which they are attached, form 3-8 membered cycloalkyl or 3-8 membered heterocycloalkyl;
R³ is selected from the group consisting of hydrogen, C₁-C₆ alkyl, OH, ON, NO₂, halogen, C₁-C₆ haloalkyl, C₁-C₆ alkoxy, C₁-C₆ alkylamine, and C₁-C₆ haloalkyl-C₃-C₆ cycloalkyl;
R⁴ is independently, at each occurrence, selected from the group consisting of hydrogen, halogen, C₁-C₆ alkyl, 0(0)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R⁵ is selected from the group consisting of hydrogen, halogen, C₁-C₆ alkyl, C₁-C₆ haloalkyl, C₁-C₆ haloalkoxy, and C₁-C₆ alkoxy;
R¹ is selected from the group consisting of:

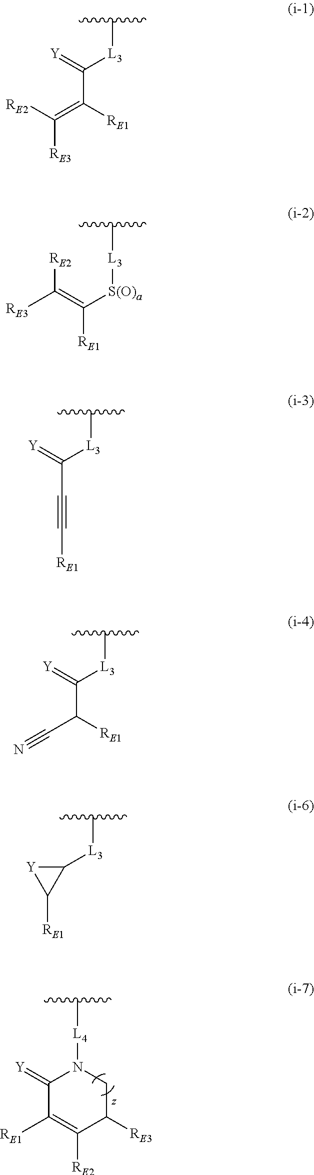

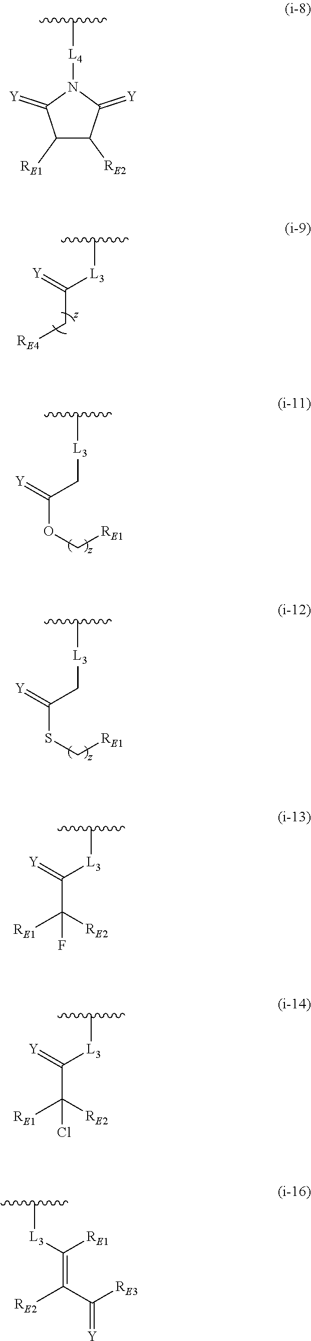

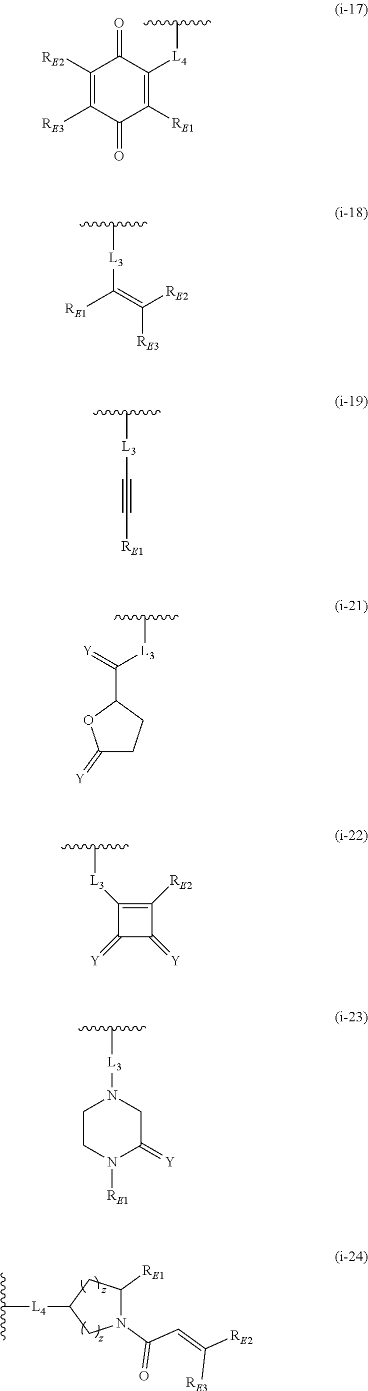

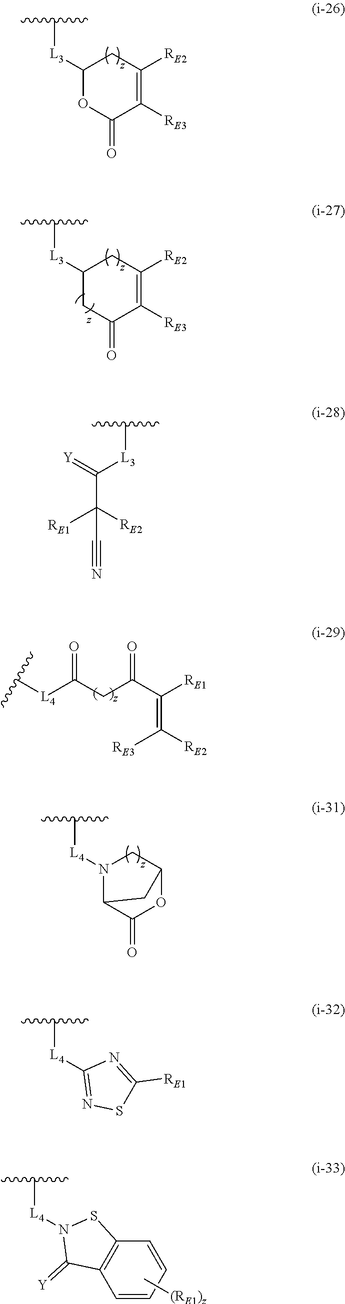

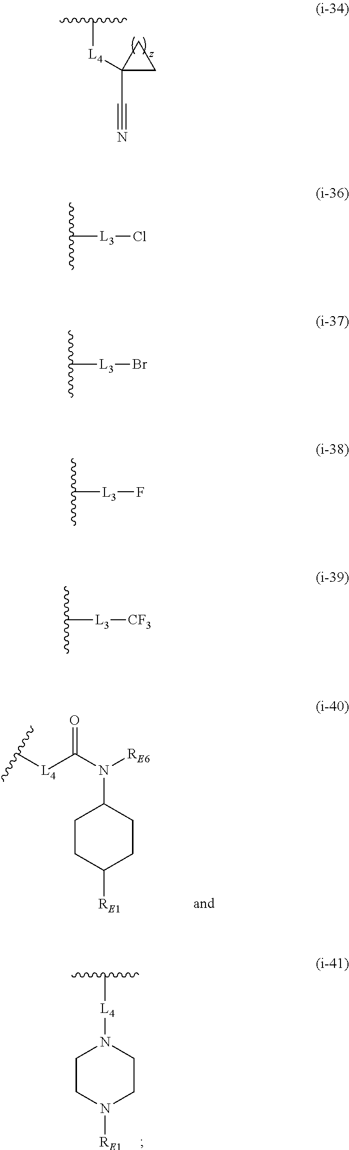

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, cis-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7};
R_{E7} is hydrogen or C₁-C₆ alkyl; and
p is 0, 1, or 2.

In an embodiment, R¹ is

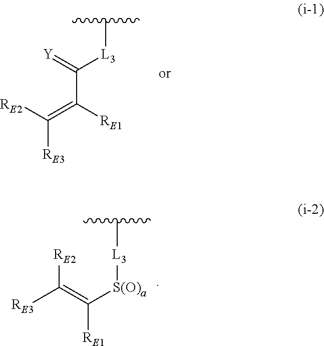

In another embodiment, R¹ is

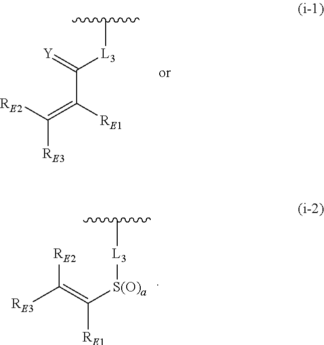

wherein
L₃ is —NH— or a bond;
Y is O;
each of R_{E1}, R_{E2}, and R_{E3} is independently selected from the group consisting of hydrogen and halogen; and
a is 2.

In yet another embodiment, R¹ is selected from the group consisting of

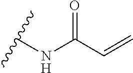 [SMILES: *NC(=O)C=C]

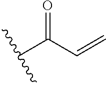 [SMILES: *C(=O)C=C]

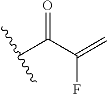 [SMILES: *C(=O)C(=C)F]

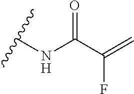 [SMILES: *NC(=O)C(=C)F]

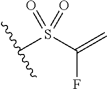 [SMILES: *S(=O)(=O)C(=C)F]

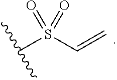 [SMILES: *S(=O)(=O)C=C]

In still another embodiment, the compound of Formula I is a compound of Formula Ib:

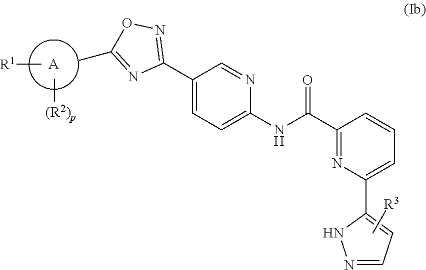

or a pharmaceutically acceptable salt thereof.

In an embodiment, the compound of Formula I is a compound of Formula Ic:

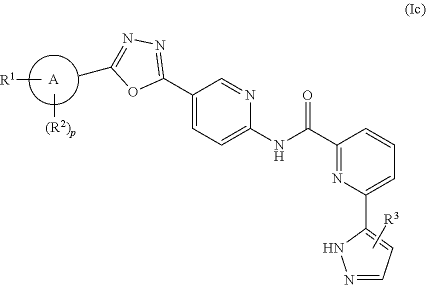

or a pharmaceutically acceptable salt thereof.

In another embodiment, A is selected from the group consisting of

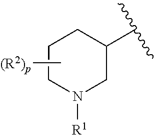

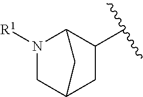

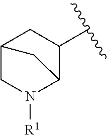

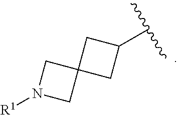

In yet another embodiment, the compound of Formula I is a compound of Formula Id:

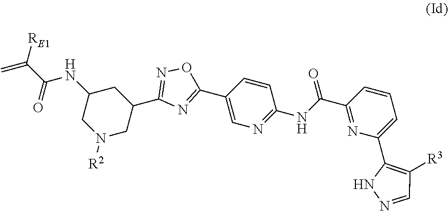

or a pharmaceutically acceptable salt thereof;
wherein R_{E} is hydrogen or halogen.

In still another embodiment, the compound of Formula I is a compound of Formula Ie:

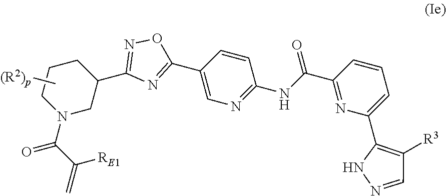

or a pharmaceutically acceptable salt thereof;
wherein R_{E1} is hydrogen or halogen.

In an embodiment, R² is halogen. In another embodiment, two R², together with the carbon atom to which they are attached, form 3-6 membered cycloalkyl.

In yet another embodiment, R³ is selected from the group consisting of hydrogen, C₁-C₃ alkyl, and halogen. In still another embodiment, R³ is hydrogen. In an embodiment, R³ is C₁-C₃ alkyl. In another embodiment, R³ is halogen.

In an embodiment, R⁵ is selected from the group consisting of C₁-C₆ alkyl, C₁-C₆ haloalkyl, and C₁-C₆ haloalkoxy. In another embodiment, R⁵ is C₁-C₆ haloalkyl. In yet another embodiment, R⁵ is C₁-C₆ haloalkoxy.

In another embodiment, the compound of Formula I is selected from the group consisting of a compound in Table 1.

TABLE 1

| Compound No. | Structure |
|---|---|
| 001 | 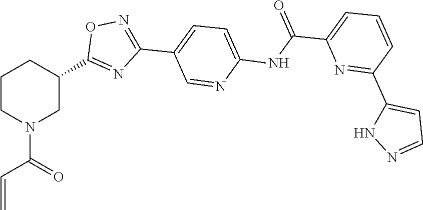 [SMILES: C=CC(=O)N1CCC[C@H](c2nc(-c3ccc(NC(=O)c4cccc(-c5ccn[nH]5)n4)nc3)no2)C1] |
| 002 | 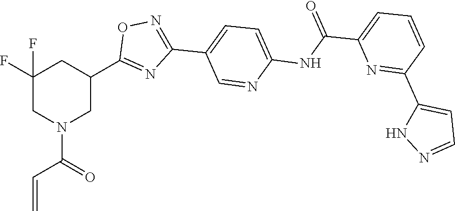 [SMILES: C=CC(=O)N1CC(c2nc(-c3ccc(NC(=O)c4cccc(-c5ccn[nH]5)n4)nc3)no2)CC(F)(F)C1] |
| 003 | 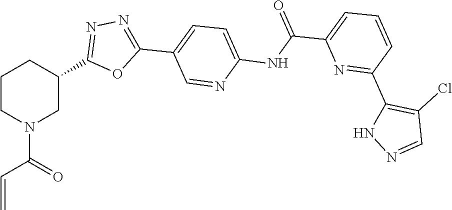 [SMILES: C=CC(=O)N1CCC[C@H](c2nnc(-c3ccc(NC(=O)c4cccc(-c5[nH]ncc5Cl)n4)nc3)o2)C1] |
| 004 | 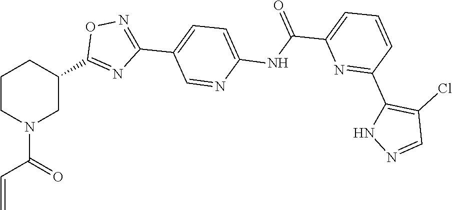 [SMILES: C=CC(=O)N1CCC[C@H](c2nc(-c3ccc(NC(=O)c4cccc(-c5[nH]ncc5Cl)n4)nc3)no2)C1] |
| 005 | 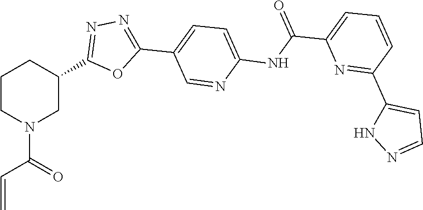 [SMILES: C=CC(=O)N1CCC[C@H](c2nnc(-c3ccc(NC(=O)c4cccc(-c5ccn[nH]5)n4)nc3)o2)C1] |
| 006 | 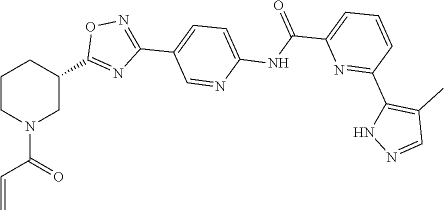 [SMILES: C=CC(=O)N1CCC[C@H](c2nc(-c3ccc(NC(=O)c4cccc(-c5[nH]ncc5C)n4)nc3)no2)C1] |
| 007 | 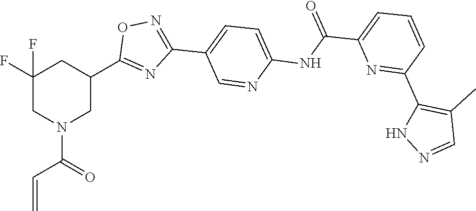 [SMILES: C=CC(=O)N1CC(c2nc(-c3ccc(NC(=O)c4cccc(-c5[nH]ncc5C)n4)nc3)no2)CC(F)(F)C1] |
| 008 | 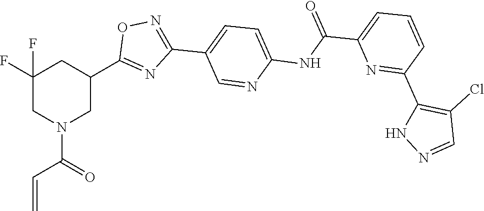 [SMILES: C=CC(=O)N1CC(c2nc(-c3ccc(NC(=O)c4cccc(-c5[nH]ncc5Cl)n4)nc3)no2)CC(F)(F)C1] |
| 009 | 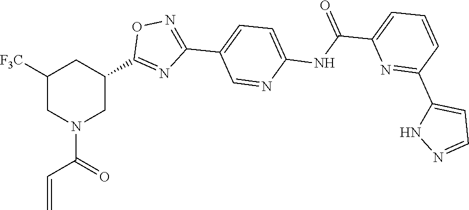 [SMILES: C=CC(=O)N1CC(C(F)(F)F)C[C@H](c2nc(-c3ccc(NC(=O)c4cccc(-c5ccn[nH]5)n4)nc3)no2)C1] |
| 010 | 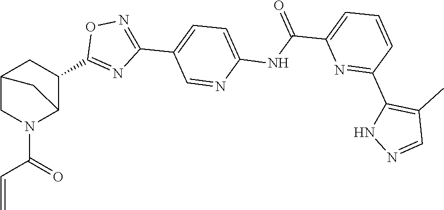 [SMILES: C=CC(=O)N1CC2CC1[C@@H](c1nc(-c3ccc(NC(=O)c4cccc(-c5[nH]ncc5C)n4)nc3)no1)C2] |
| 011 | 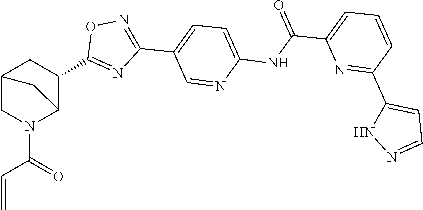 [SMILES: C=CC(=O)N1CC2CC1[C@@H](c1nc(-c3ccc(NC(=O)c4cccc(-c5ccn[nH]5)n4)nc3)no1)C2] |
| 012 | 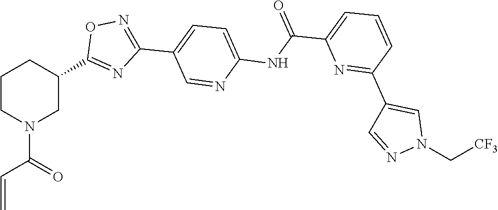 [SMILES: C=CC(=O)N1CCC[C@H](c2nc(-c3ccc(NC(=O)c4cccc(-c5cnn(CC(F)(F)F)c5)n4)nc3)no2)C1] |
| 013 | 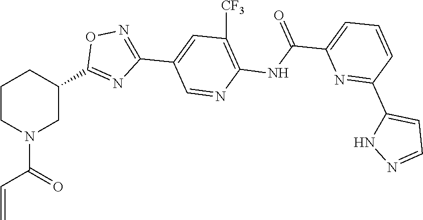 [SMILES: C=CC(=O)N1CCC[C@H](c2nc(-c3cnc(NC(=O)c4cccc(-c5ccn[nH]5)n4)c(C(F)(F)F)c3)no2)C1] |
| 014 | 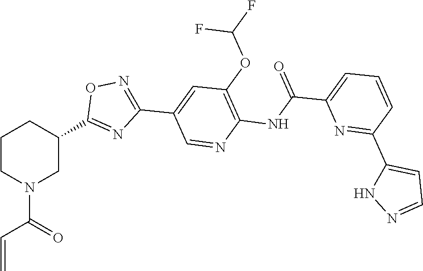 [SMILES: C=CC(=O)N1CCC[C@H](c2nc(-c3cnc(NC(=O)c4cccc(-c5ccn[nH]5)n4)c(OC(F)F)c3)no2)C1] |
| 015 | 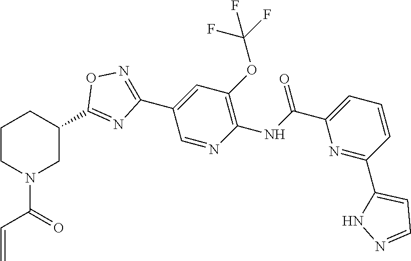 [SMILES: C=CC(=O)N1CCC[C@H](c2nc(-c3cnc(NC(=O)c4cccc(-c5ccn[nH]5)n4)c(OC(F)(F)F)c3)no2)C1] |
| 016 | 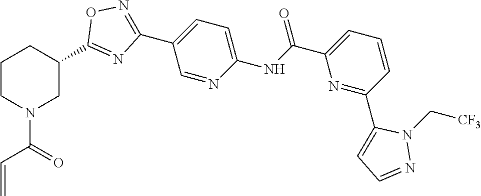 [SMILES: C=CC(=O)N1CCC[C@H](c2nc(-c3ccc(NC(=O)c4cccc(-c5ccnn5CC(F)(F)F)n4)nc3)no2)C1] |
| 017 | 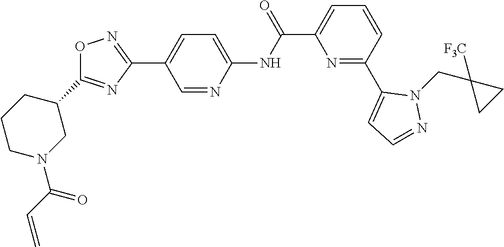 [SMILES: C=CC(=O)N1CCC[C@H](c2nc(-c3ccc(NC(=O)c4cccc(-c5ccnn5CC5(C(F)(F)F)CC5)n4)nc3)no2)C1] |

or a pharmaceutically acceptable salt thereof.

In another aspect, provided herein is a compound of Formula II:

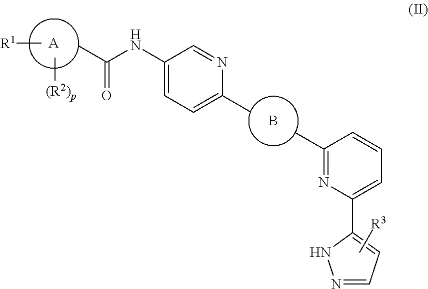

or a pharmaceutically acceptable salt thereof;
   wherein
A is 3-8 membered heterocycloalkyl;
B is selected from the group consisting of triazolyl, oxadiazolyl, oxazolyl, thiazolyl, imidazolyl, and isothiazolopyridinylamino;
R² is independently, at each occurrence, selected from the group consisting of halogen, C₁-C₆ alkyl, C₁-C₆ haloalkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, 5-10 membered heteroaryl, OR⁴, N(R⁴)₂, SO₂R⁴, and SR⁴;
alternatively, two R², together with the carbon atom to which they are attached, form 3-8 membered cycloalkyl or 3-8 membered heterocycloalkyl;
R³ is selected from the group consisting of hydrogen, C₁-C₆ alkyl, OH, ON, NO₂, halogen, C₁-C₆ haloalkyl, C₁-C₆ alkoxy, and C₁-C₆ alkylamine;
R⁴ is independently, at each occurrence, selected from the group consisting of hydrogen, halogen, C₁-C₆ alkyl, 0(0)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R¹ is selected from the group consisting of:

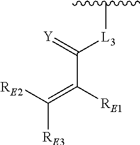

(i-1)

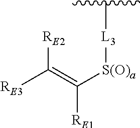

(i-2)

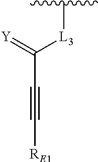

(i-3)

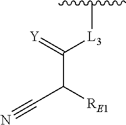

(i-4)

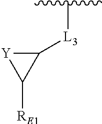

(i-6)

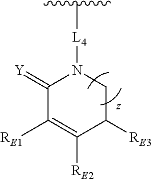

(i-7)

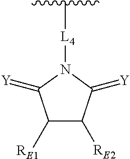

(i-8)

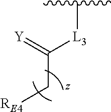

(i-9)

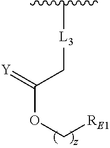

(i-11)

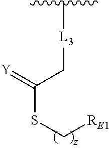

(i-12)

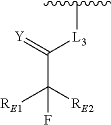

(i-13)

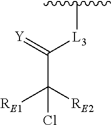

(i-14)

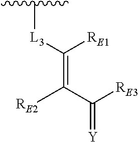

(i-16)

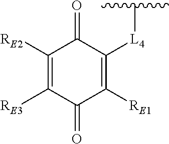

(i-17)

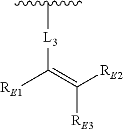

(i-18)

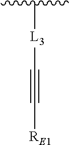

(i-19)

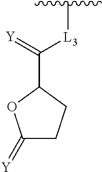

(i-21)

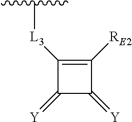

(i-22)

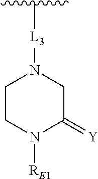

(i-23)

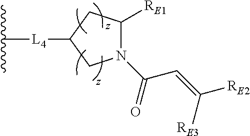

(i-24)

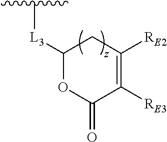

(i-26)

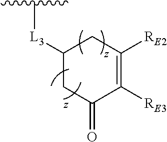

(i-27)

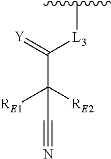

(i-28)

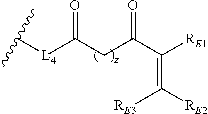

(i-29)

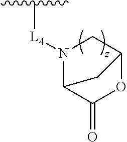

(i-31)

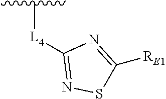

(i-32)

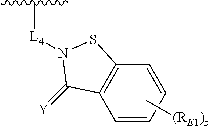

(i-33)

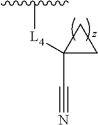

(i-34)

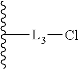

(i-36)

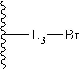

(i-37)

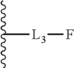

(i-38)

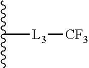

(i-39)

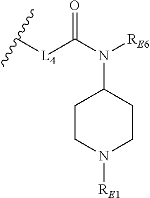

(i-40)
and

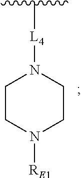

(i-41)

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, ciS-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7};
R_{E7} is hydrogen or C₁-C₆ alkyl; and
p is 0, 1, or 2.

In an aspect of Formula II, B is selected from the group consisting of triazolyl, oxadiazolyl, oxazolyl, thiazolyl, imidazolyl, isothiazolopyridinylamino, and pyrazolyl; and the remaining variables are defined supra.

In an embodiment, R¹ is

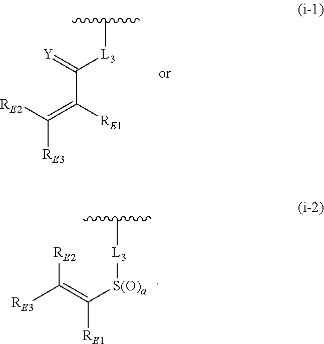

In another embodiment, R¹ is

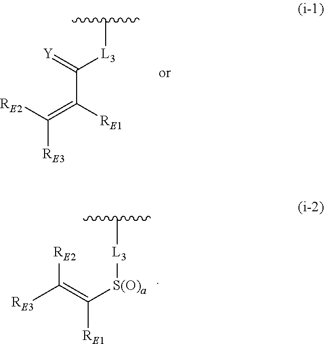

wherein
L₃ is —NH— or a bond;
Y is O;
each of R_{E}, R_{E}2, and R_{E3} is independently selected from the group consisting of hydrogen and halogen; and
a is 2.

In yet another embodiment, R¹ is selected from the group consisting of

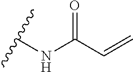 [SMILES: *NC(=O)C=C]

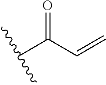 [SMILES: *C(=O)C=C]

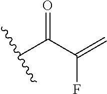 [SMILES: *C(=O)C(=C)F]

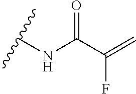 [SMILES: *NC(=O)C(=C)F]

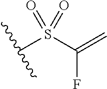 [SMILES: *S(=O)(=O)C(=C)F]

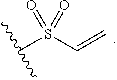 [SMILES: *S(=O)(=O)C=C]

In still another embodiment, the compound of Formula II is a compound of Formula IIa:

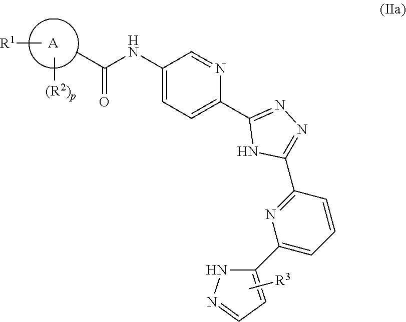

or a pharmaceutically acceptable salt thereof.

In an embodiment, the compound of Formula II is a compound of Formula IIb:

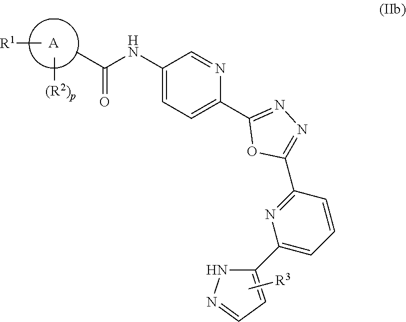

or a pharmaceutically acceptable salt thereof.

In another embodiment, the compound of Formula II is a compound of Formula IIc:

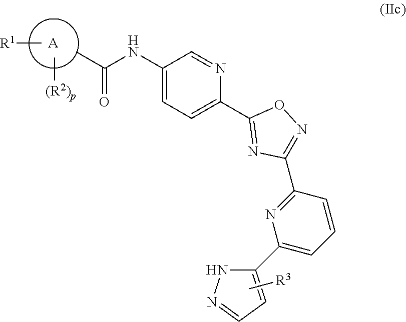

or a pharmaceutically acceptable salt thereof.

In yet another embodiment, the compound of Formula II is a compound of Formula IId:

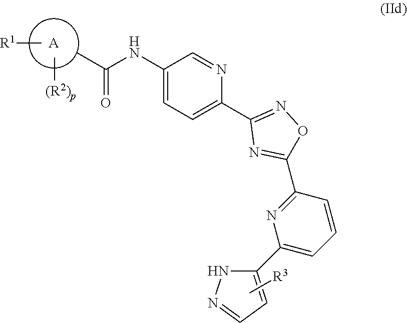

or a pharmaceutically acceptable salt thereof.

In still another embodiment, the compound of Formula II is a compound of Formula IIe:

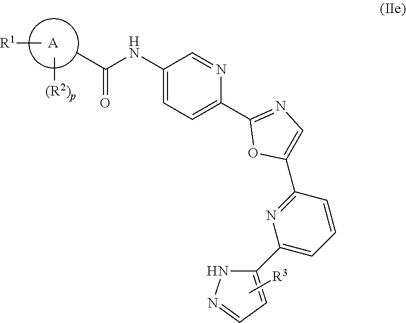

or a pharmaceutically acceptable salt thereof.

In an embodiment, the compound of Formula II is a compound of Formula IIf:

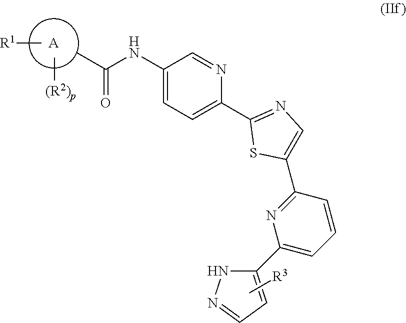

or a pharmaceutically acceptable salt thereof.

In another embodiment, the compound of Formula II is a compound of Formula IIg:

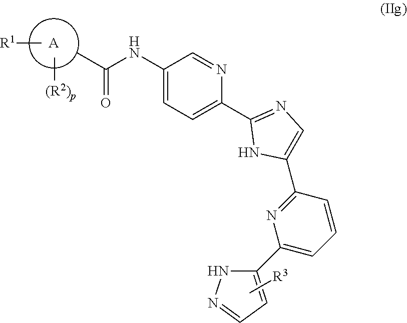

or a pharmaceutically acceptable salt thereof.

In yet another embodiment, R² is halogen. In still another embodiment, two R², together with the carbon atom to which they are attached, form 3-6 membered cycloalkyl.

In an embodiment, R³ is selected from the group consisting of hydrogen, C₁-C₃ alkyl, and halogen. In another embodiment, R³ is hydrogen. In yet another embodiment, R³ is C₁-C₃ alkyl. In still another embodiment, R³ is halogen.

In yet another embodiment, the moiety:

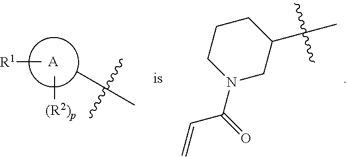

In an embodiment, the compound of Formula II is selected from the group consisting of a compound in Table 2.

TABLE 2

| Compound No. | Structure |
|---|---|
| 018 | 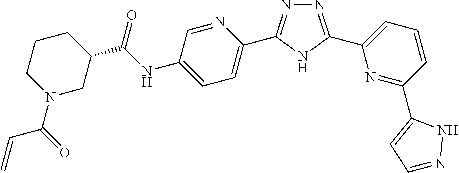 [SMILES: C=CC(=O)N1CCC[C@H](C(=O)Nc2ccc(-c3nnc(-c4cccc(-c5ccn[nH]5)n4)[nH]3)nc2)C1] |
| 019 | 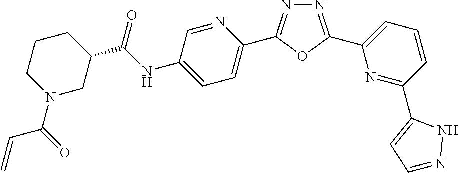 [SMILES: C=CC(=O)N1CCC[C@H](C(=O)Nc2ccc(-c3nnc(-c4cccc(-c5ccn[nH]5)n4)o3)nc2)C1] |
| 020 | 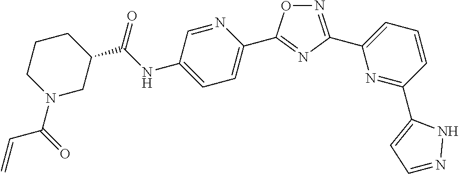 [SMILES: C=CC(=O)N1CCC[C@H](C(=O)Nc2ccc(-c3nc(-c4cccc(-c5ccn[nH]5)n4)no3)nc2)C1] |
| 021 | 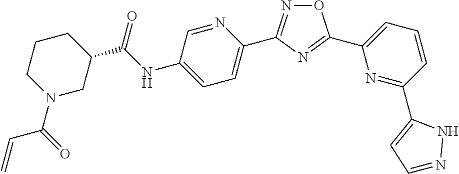 [SMILES: C=CC(=O)N1CCC[C@H](C(=O)Nc2ccc(-c3noc(-c4cccc(-c5ccn[nH]5)n4)n3)nc2)C1] |
| 022 | 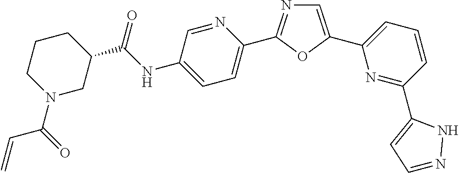 [SMILES: C=CC(=O)N1CCC[C@H](C(=O)Nc2ccc(-c3ncc(-c4cccc(-c5ccn[nH]5)n4)o3)nc2)C1] |
| 023 | 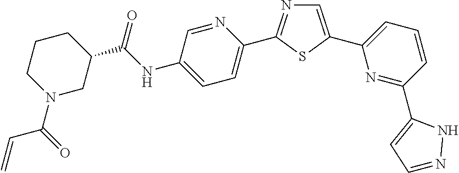 [SMILES: C=CC(=O)N1CCC[C@H](C(=O)Nc2ccc(-c3ncc(-c4cccc(-c5ccn[nH]5)n4)s3)nc2)C1] |
| 024 | 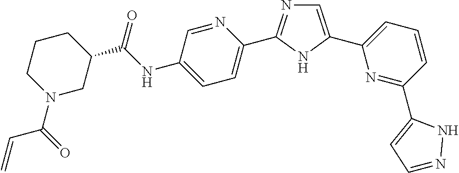 [SMILES: C=CC(=O)N1CCC[C@H](C(=O)Nc2ccc(-c3ncc(-c4cccc(-c5ccn[nH]5)n4)[nH]3)nc2)C1] |
| 025 | 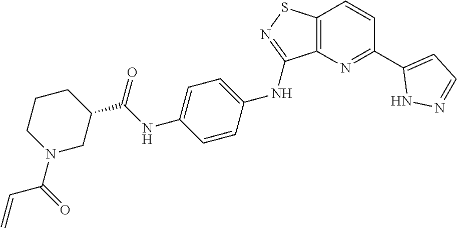 [SMILES: C=CC(=O)N1CCC[C@H](C(=O)Nc2ccc(Nc3nsc4ccc(-c5ccn[nH]5)nc34)cc2)C1] |
| 026 | 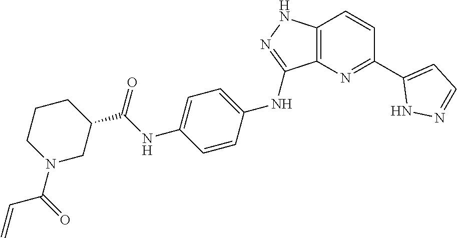 [SMILES: C=CC(=O)N1CCC[C@H](C(=O)Nc2ccc(Nc3n[nH]c4ccc(-c5ccn[nH]5)nc34)cc2)C1] |

or a pharmaceutically acceptable salt thereof.

In another embodiment, the compound of Formula II is selected from the group consisting of a compound in Table 2a.

TABLE 2a

| Compound No. | Structure |
|---|---|
| 056 | 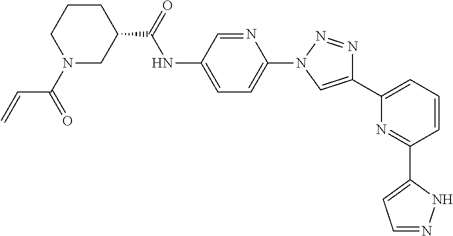 [SMILES: C=CC(=O)N1CCC[C@H](C(=O)Nc2ccc(-n3cc(-c4cccc(-c5ccn[nH]5)n4)nn3)nc2)C1] |
| 057 | 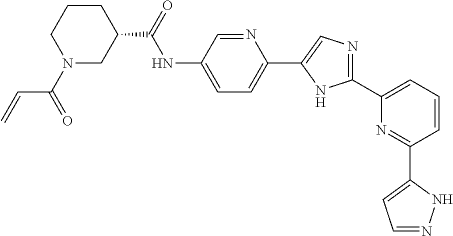 [SMILES: C=CC(=O)N1CCC[C@H](C(=O)Nc2ccc(-c3cnc(-c4cccc(-c5ccn[nH]5)n4)[nH]3)nc2)C1] |
| 058 | 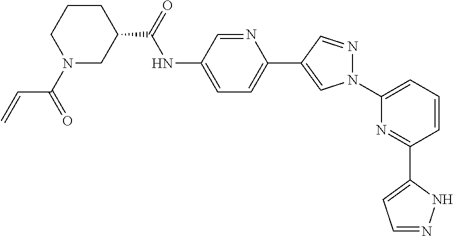 [SMILES: C=CC(=O)N1CCC[C@H](C(=O)Nc2ccc(-c3cnn(-c4cccc(-c5ccn[nH]5)n4)c3)nc2)C1] |
| 059 | 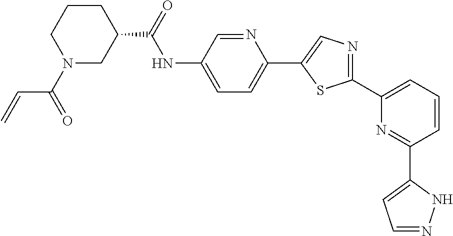 [SMILES: C=CC(=O)N1CCC[C@H](C(=O)Nc2ccc(-c3cnc(-c4cccc(-c5ccn[nH]5)n4)s3)nc2)C1] |
| 060 | 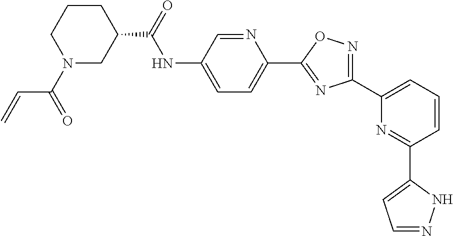 [SMILES: C=CC(=O)N1CCC[C@H](C(=O)Nc2ccc(-c3nc(-c4cccc(-c5ccn[nH]5)n4)no3)nc2)C1] |

or a pharmaceutically acceptable salt thereof.

In yet another aspect, provided herein is a compound of Formula III:

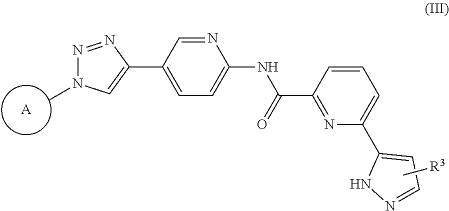

or a pharmaceutically acceptable salt thereof;
   wherein
A is selected from the group consisting of

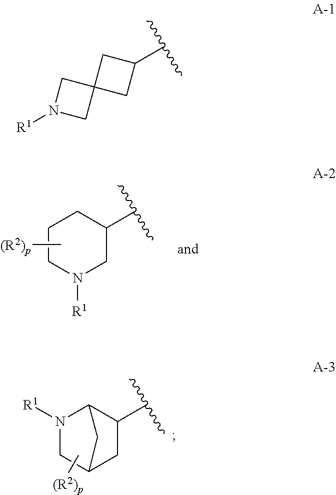

R² is independently, at each occurrence, halogen;
alternatively, two R², together with the carbon atom to which they are attached, form 3-8 membered cycloalkyl or 3-8 membered heterocycloalkyl;
R³ is selected from the group consisting of hydrogen, C₁-C₆ alkyl, and halogen;
provided when A is A-1, R³ is not hydrogen;
R¹ is selected from the group consisting of:

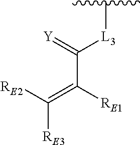

(i-1)

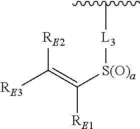

(i-2)

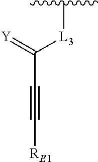

(i-3)

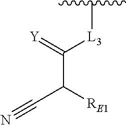

(i-4)

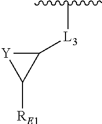

(i-6)

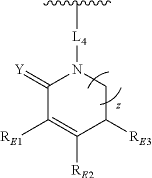

(i-7)

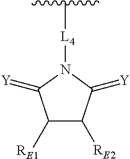

(i-8)

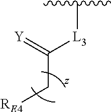

(i-9)

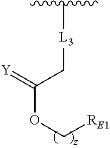

(i-11)

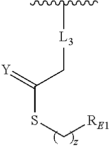

(i-12)

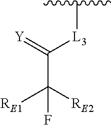

(i-13)

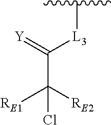

(i-14)

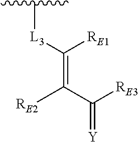

(i-16)

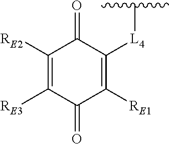

(i-17)

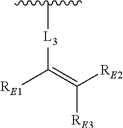

(i-18)

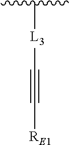

(i-19)

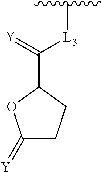

(i-21)

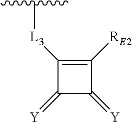

(i-22)

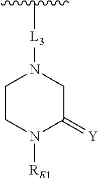

(i-23)

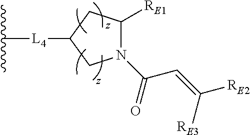

(i-24)

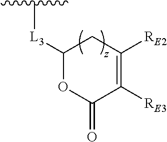

(i-26)

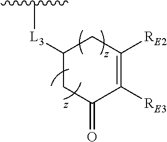

(i-27)

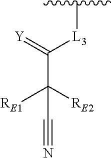

(i-28)

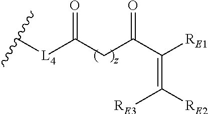

(i-29)

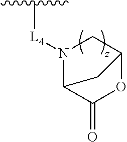

(i-31)

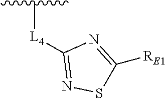

(i-32)

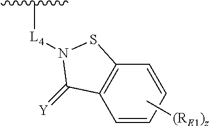

(i-33)

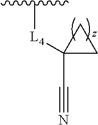

(i-34)

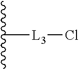

(i-36)

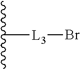

(i-37)

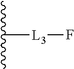

(i-38)

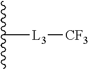

(i-39)

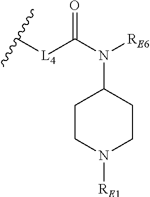

(i-40)
and

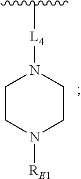

(i-41)

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, ciS-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E} and R_{E3}, or R_{E2} and R_{E3}, or R_{E} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7};
R_{E7} is hydrogen or C₁-C₆ alkyl; and
p is 0, 1, or 2.

In an embodiment, R¹ is

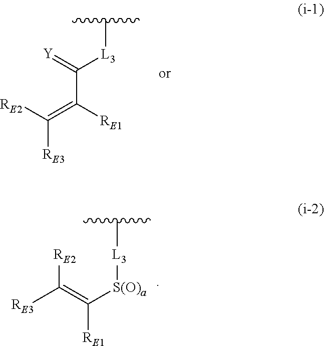

In another embodiment, R¹ is

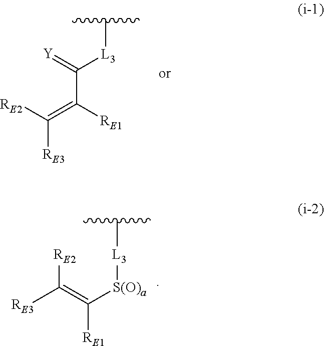

wherein
L₃ is —NH— or a bond;
Y is O;
each of R_{E}, R_{E2}, and R_{E3} is independently selected from the group consisting of hydrogen and halogen; and
a is 2.

In yet another embodiment, R¹ is selected from the group consisting of

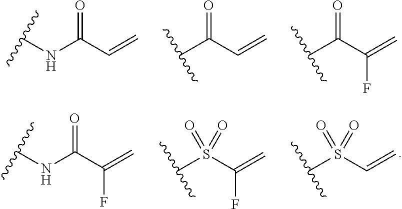

In still another embodiment, p is 2.

In another embodiment, R² is halogen. In yet another embodiment, two R², together with the carbon atom to which they are attached, form 3-6 membered cycloalkyl.

In still another embodiment, p is 2, and R² is halogen. In an embodiment, p is 2, and two R², together with the carbon atom to which they are attached, form 3-6 membered cycloalkyl.

In another embodiment, the compound of Formula III is selected from the group consisting of a compound in Table 3.

TABLE 3

| Compound No. | Structure |
|---|---|
| 027 | 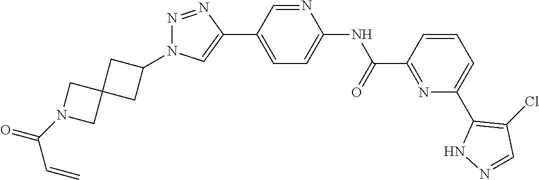 [SMILES: C=CC(=O)N1CC2(CC(n3cc(-c4ccc(NC(=O)c5cccc(-c6[nH]ncc6Cl)n5)nc4)nn3)C2)C1] |
| 028 | 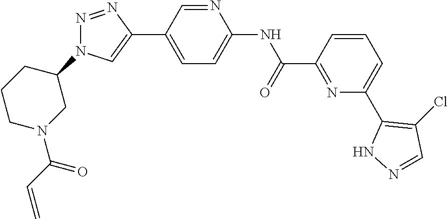 [SMILES: C=CC(=O)N1CCC[C@@H](n2cc(-c3ccc(NC(=O)c4cccc(-c5[nH]ncc5Cl)n4)nc3)nn2)C1] |
| 029 | 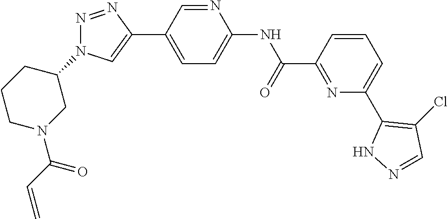 [SMILES: C=CC(=O)N1CCC[C@H](n2cc(-c3ccc(NC(=O)c4cccc(-c5[nH]ncc5Cl)n4)nc3)nn2)C1] |
| 030 | 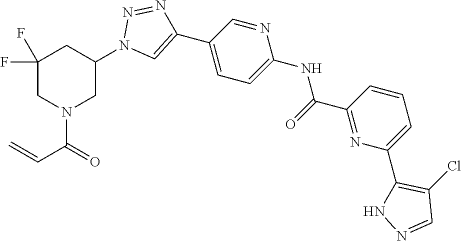 [SMILES: C=CC(=O)N1CC(n2cc(-c3ccc(NC(=O)c4cccc(-c5[nH]ncc5Cl)n4)nc3)nn2)CC(F)(F)C1] |
| 031 | 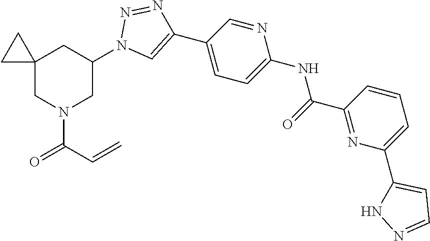 [SMILES: C=CC(=O)N1CC(n2cc(-c3ccc(NC(=O)c4cccc(-c5ccn[nH]5)n4)nc3)nn2)CC2(CC2)C1] |
| 032 | 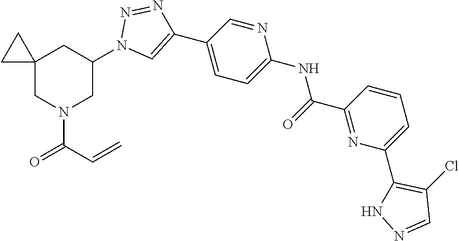 [SMILES: C=CC(=O)N1CC(n2cc(-c3ccc(NC(=O)c4cccc(-c5[nH]ncc5Cl)n4)nc3)nn2)CC2(CC2)C1] |
| 033 | 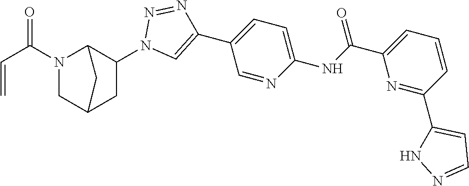 [SMILES: C=CC(=O)N1CC2CC1C(n1cc(-c3ccc(NC(=O)c4cccc(-c5ccn[nH]5)n4)nc3)nn1)C2] |
| 034 | 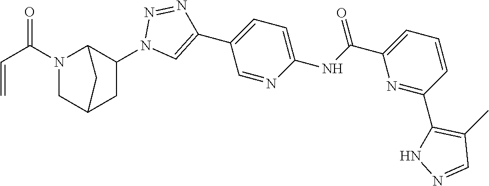 [SMILES: C=CC(=O)N1CC2CC1C(n1cc(-c3ccc(NC(=O)c4cccc(-c5[nH]ncc5C)n4)nc3)nn1)C2] |
| 035 | 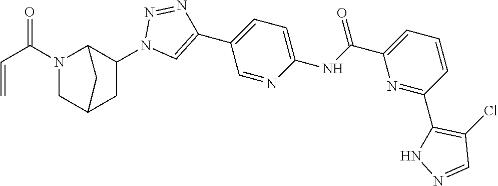 [SMILES: C=CC(=O)N1CC2CC1C(n1cc(-c3ccc(NC(=O)c4cccc(-c5[nH]ncc5Cl)n4)nc3)nn1)C2] |
| 036 | 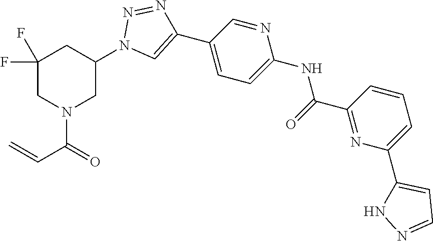 [SMILES: C=CC(=O)N1CC(n2cc(-c3ccc(NC(=O)c4cccc(-c5ccn[nH]5)n4)nc3)nn2)CC(F)(F)C1] |

or a pharmaceutically acceptable salt thereof.

In another aspect, provided herein is a compound selected from Table 4.

TABLE 4

| Compound No. | Structure |
|---|---|
| 037 | 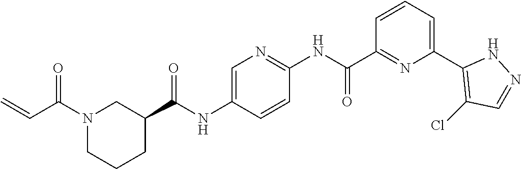 [SMILES: C=CC(=O)N1CCC[C@H](C(=O)Nc2ccc(NC(=O)c3cccc(-c4[nH]ncc4Cl)n3)nc2)C1] |
| 038 | 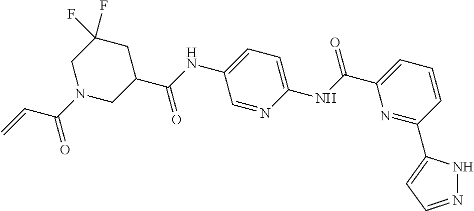 [SMILES: C=CC(=O)N1CC(C(=O)Nc2ccc(NC(=O)c3cccc(-c4ccn[nH]4)n3)nc2)CC(F)(F)C1] |
| 039 | 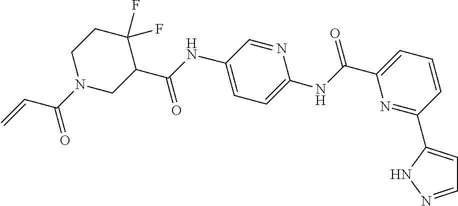 [SMILES: C=CC(=O)N1CCC(F)(F)C(C(=O)Nc2ccc(NC(=O)c3cccc(-c4ccn[nH]4)n3)nc2)C1] |
| 040 | 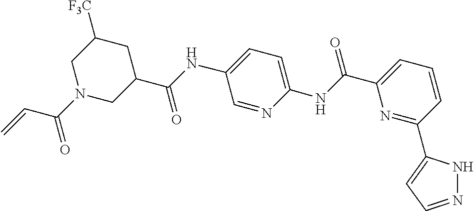 [SMILES: C=CC(=O)N1CC(C(=O)Nc2ccc(NC(=O)c3cccc(-c4ccn[nH]4)n3)nc2)CC(C(F)(F)F)C1] |
| 041 | 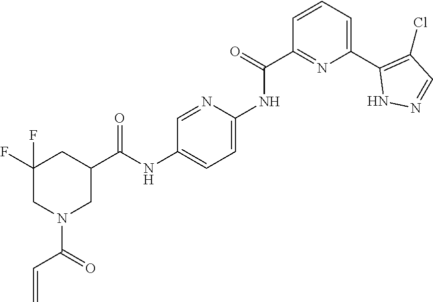 [SMILES: C=CC(=O)N1CC(C(=O)Nc2ccc(NC(=O)c3cccc(-c4[nH]ncc4Cl)n3)nc2)CC(F)(F)C1] |
| 042 | 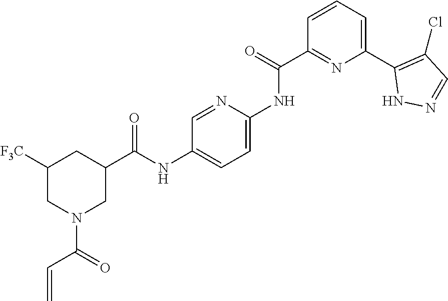 [SMILES: C=CC(=O)N1CC(C(=O)Nc2ccc(NC(=O)c3cccc(-c4[nH]ncc4Cl)n3)nc2)CC(C(F)(F)F)C1] |
| 043 | 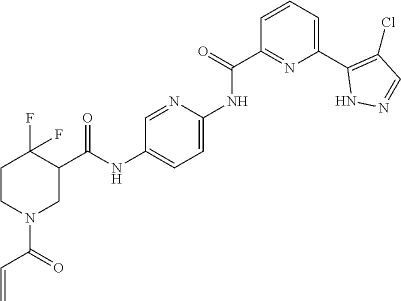 [SMILES: C=CC(=O)N1CCC(F)(F)C(C(=O)Nc2ccc(NC(=O)c3cccc(-c4[nH]ncc4Cl)n3)nc2)C1] |
| 044 | 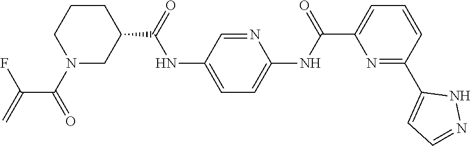 [SMILES: C=C(F)C(=O)N1CCC[C@H](C(=O)Nc2ccc(NC(=O)c3cccc(-c4ccn[nH]4)n3)nc2)C1] |
| 045 | 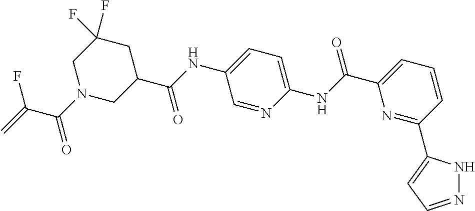 [SMILES: C=C(F)C(=O)N1CC(C(=O)Nc2ccc(NC(=O)c3cccc(-c4ccn[nH]4)n3)nc2)CC(F)(F)C1] |
| 046 | 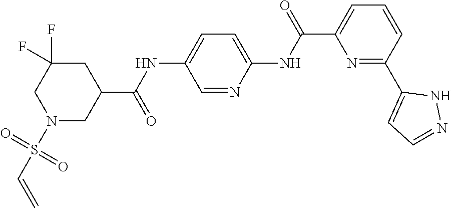 [SMILES: C=CS(=O)(=O)N1CC(C(=O)Nc2ccc(NC(=O)c3cccc(-c4ccn[nH]4)n3)nc2)CC(F)(F)C1] |
| 047 | 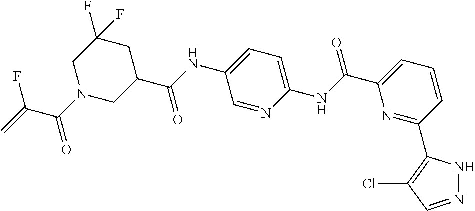 [SMILES: C=C(F)C(=O)N1CC(C(=O)Nc2ccc(NC(=O)c3cccc(-c4[nH]ncc4Cl)n3)nc2)CC(F)(F)C1] |
| 048 | 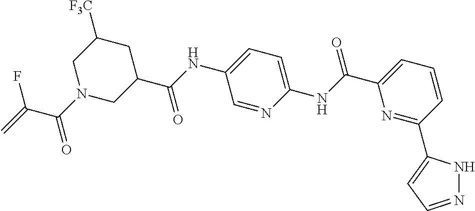 [SMILES: C=C(F)C(=O)N1CC(C(=O)Nc2ccc(NC(=O)c3cccc(-c4ccn[nH]4)n3)nc2)CC(C(F)(F)F)C1] |
| 049 | 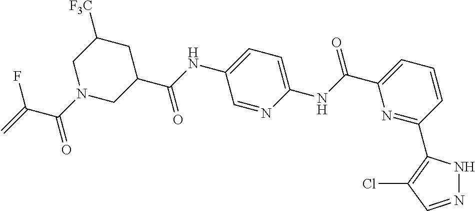 [SMILES: C=C(F)C(=O)N1CC(C(=O)Nc2ccc(NC(=O)c3cccc(-c4[nH]ncc4Cl)n3)nc2)CC(C(F)(F)F)C1] |
| 050 | 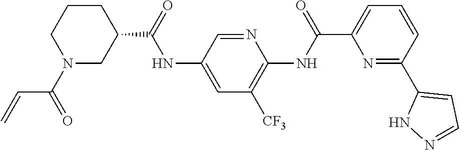 [SMILES: C=CC(=O)N1CCC[C@H](C(=O)Nc2cnc(NC(=O)c3cccc(-c4ccn[nH]4)n3)c(C(F)(F)F)c2)C1] |

or a pharmaceutically acceptable salt thereof.

In another aspect, provided herein is a compound of Formula IV:

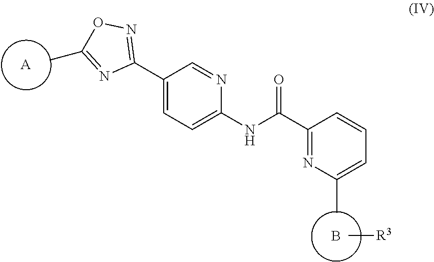

or a pharmaceutically acceptable salt thereof;
   wherein
A is selected from the group consisting of

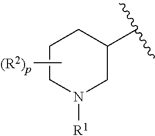

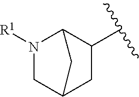

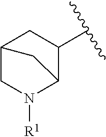

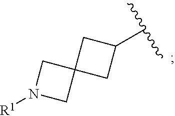

B is pyrazolyl or thiazolyl, both of which are optionally substituted with R³;
R² is independently, at each occurrence, selected from the group consisting of halogen, C₁-C₆ alkyl, C₁-C₆ haloalkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, 5-10 membered heteroaryl, OR⁴, N(R⁴)₂, SO₂R⁴, and SR⁴;
alternatively, two R², together with the carbon atom to which they are attached, form 3-8 membered cycloalkyl or 3-8 membered heterocycloalkyl;
R⁴ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl; and
R¹ is selected from the group consisting of

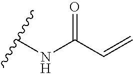

In an embodiment, the compound of Formula IV is a compound of Formula IVa:

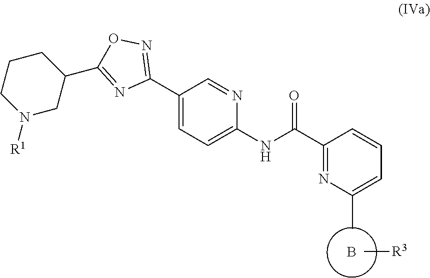

or a pharmaceutically acceptable salt thereof.

In another embodiment, the compound of Formula IV is a compound of Formula IVb:

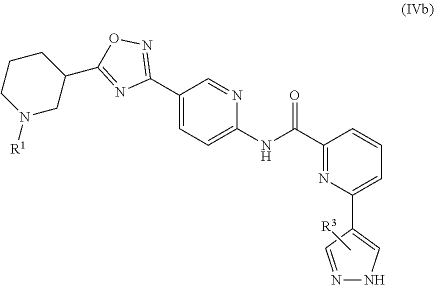

or a pharmaceutically acceptable salt thereof.

In yet another embodiment, B is thiazolyl.

In still another embodiment, the compound of Formula IV is selected from a compound in Table 5.

TABLE 5

| | |
|---|---|
| 051 | 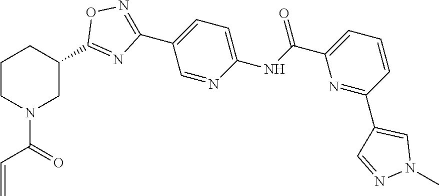 [SMILES: C=CC(=O)N1CCC[C@H](c2nc(-c3ccc(NC(=O)c4cccc(-c5cnn(C)c5)n4)nc3)no2)C1] |
| 052 | 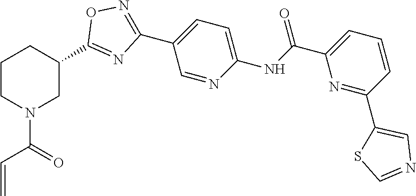 [SMILES: C=CC(=O)N1CCC[C@H](c2nc(-c3ccc(NC(=O)c4cccc(-c5cncs5)n4)nc3)no2)C1] |
| 053 | 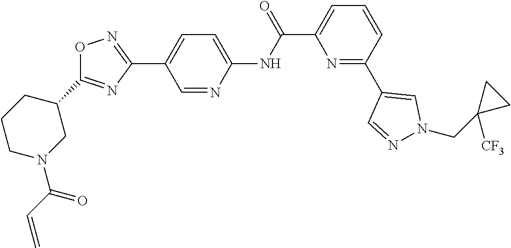 [SMILES: C=CC(=O)N1CCC[C@H](c2nc(-c3ccc(NC(=O)c4cccc(-c5cnn(CC6(C(F)(F)F)CC6)c5)n4)nc3)no2)C1] |
| 054 | 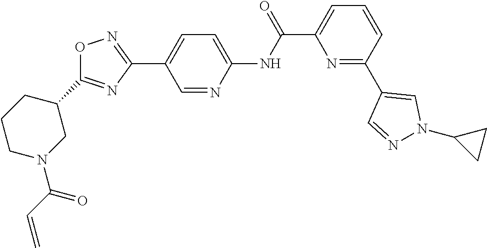 [SMILES: C=CC(=O)N1CCC[C@H](c2nc(-c3ccc(NC(=O)c4cccc(-c5cnn(C6CC6)c5)n4)nc3)no2)C1] |
| 055 | 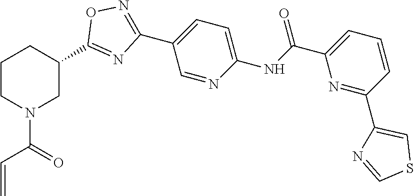 [SMILES: C=CC(=O)N1CCC[C@H](c2nc(-c3ccc(NC(=O)c4cccc(-c5cscn5)n4)nc3)no2)C1] |

or a pharmaceutically acceptable salt thereof.

In another embodiment, the compound of Formula IV is selected from a compound in Table 5a.

TABLE 5a

| | |
|---|---|
| 061 | 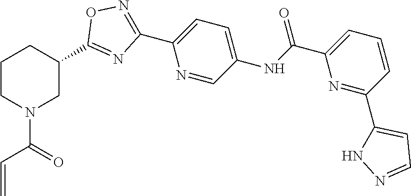 [SMILES: C=CC(=O)N1CCC[C@H](c2nc(-c3ccc(NC(=O)c4cccc(-c5ccn[nH]5)n4)cn3)no2)C1] |
| 062 | 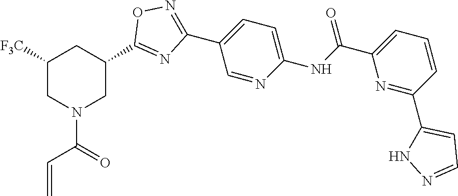 [SMILES: C=CC(=O)N1C[C@@H](c2nc(-c3ccc(NC(=O)c4cccc(-c5ccn[nH]5)n4)nc3)no2)C[C@@H](C(F)(F)F)C1] |
| 063 | 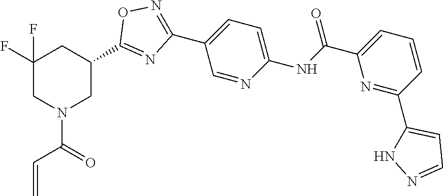 [SMILES: C=CC(=O)N1C[C@@H](c2nc(-c3ccc(NC(=O)c4cccc(-c5ccn[nH]5)n4)nc3)no2)CC(F)(F)C1] |
| 064 | 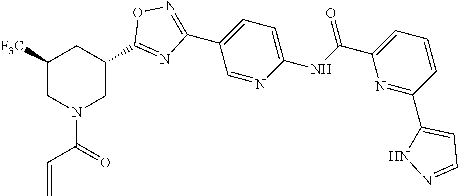 [SMILES: C=CC(=O)N1C[C@@H](c2nc(-c3ccc(NC(=O)c4cccc(-c5ccn[nH]5)n4)nc3)no2)C[C@H](C(F)(F)F)C1] |
| 065 | 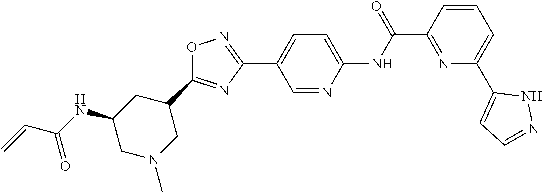 |
| 066 | 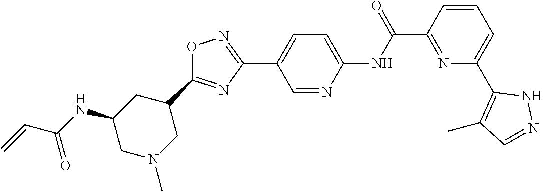 |
| 067 | 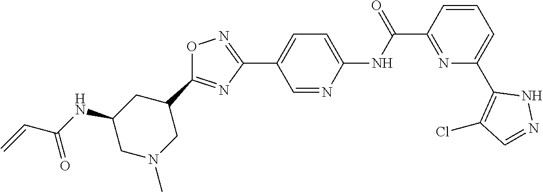 |

or a pharmaceutically acceptable salt thereof.

In yet another aspect, provided herein is a compound of Formula V:

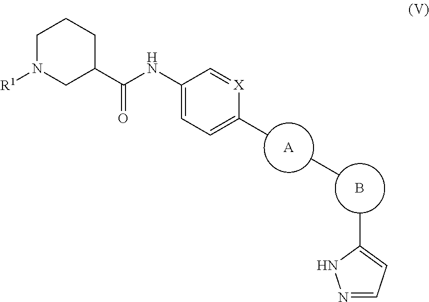

or a pharmaceutically acceptable salt thereof;
   wherein
X is N or CH;
A is selected from the group consisting of —NHC(O)—, triazolyl, imidazolyl, pyrazolyl, and oxadiazolyl;
B is thiazolyl or pyridinyl;
R¹ is selected from the group consisting of

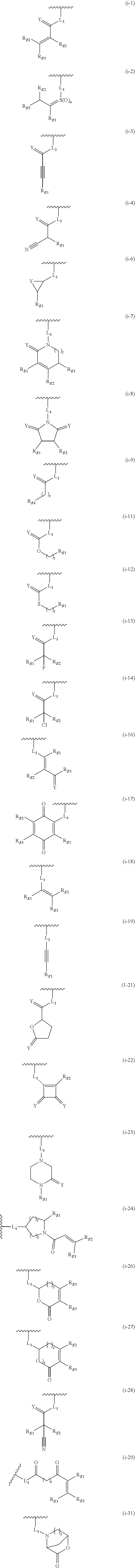

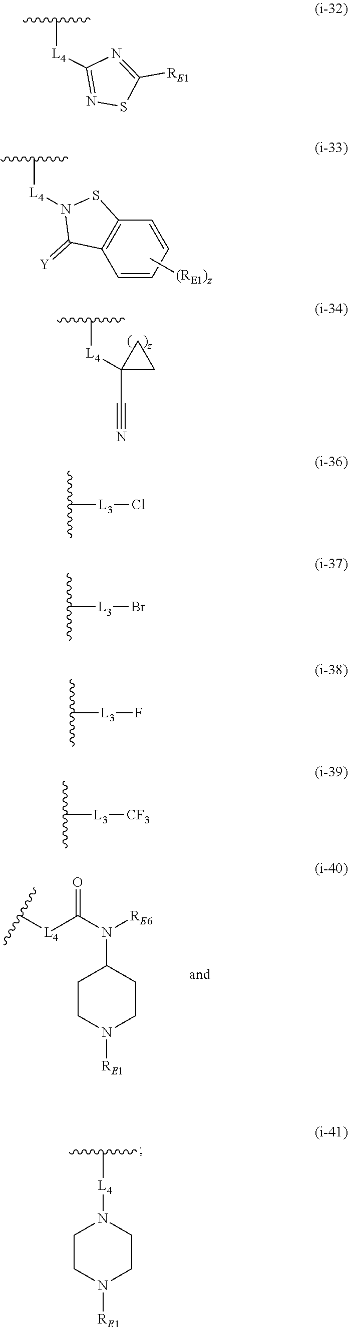

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}, trans-CR_{L3b}═CR_{L3b}—, cis-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E1}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7}; and
R_{E7} is hydrogen or C₁-C₆ alkyl.

In an embodiment, X is N. In another embodiment, X is CH.

In yet another embodiment, the compound of Formula V is a compound of Formula Va:

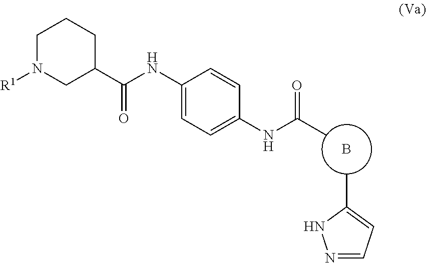

or a pharmaceutically acceptable salt thereof.

In still another embodiment, A is selected from the group consisting of

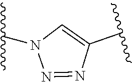 [SMILES: *c1cn(*)nn1]

In an embodiment, the compound of Formula V is a compound of Formula Vb:
or a pharmaceutically acceptable salt thereof.

In an embodiment, R¹ is

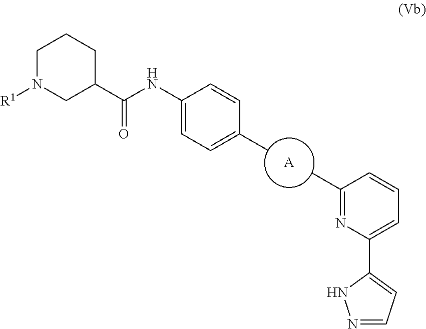

or

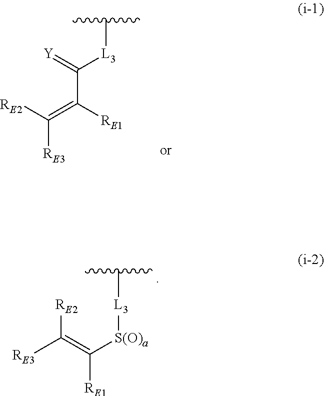

In another embodiment, R¹ is

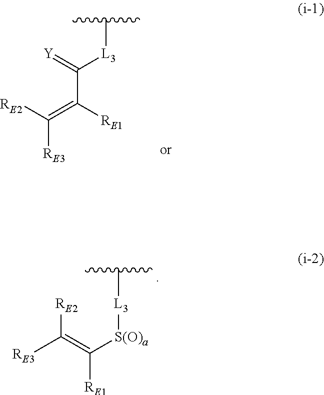

or
wherein
L₃ is —NH— or a bond;
Y is O;
each of R_{E}, R_{E2}, and R_{E3} is independently selected from the group consisting of hydrogen and halogen; and
a is 2.

In yet another embodiment, R¹ is selected from the group consisting of

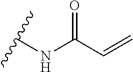 [SMILES: *NC(=O)C=C]

In still another embodiment, the compound of Formula V is selected from the group consisting of a compound in Table 6.

TABLE 6

| Compound No. | Structure |
|---|---|
| 068 | 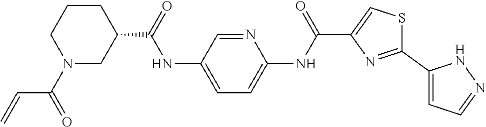 [SMILES: C=CC(=O)N1CCC[C@H](C(=O)Nc2ccc(NC(=O)c3csc(-c4ccn[nH]4)n3)nc2)C1] |
| 069 | 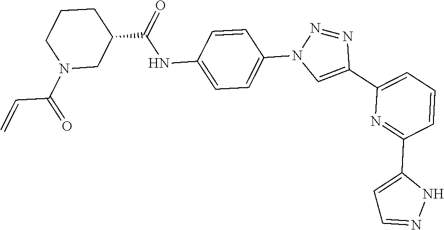 [SMILES: C=CC(=O)N1CCC[C@H](C(=O)Nc2ccc(-n3cc(-c4cccc(-c5ccn[nH]5)n4)nn3)cc2)C1] |
| 070 | 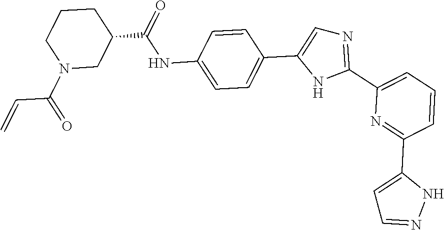 [SMILES: C=CC(=O)N1CCC[C@H](C(=O)Nc2ccc(-c3cnc(-c4cccc(-c5ccn[nH]5)n4)[nH]3)cc2)C1] |
| 071 | 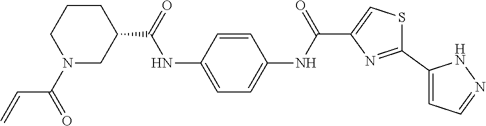 [SMILES: C=CC(=O)N1CCC[C@H](C(=O)Nc2ccc(NC(=O)c3csc(-c4ccn[nH]4)n3)cc2)C1] |
| 072 | 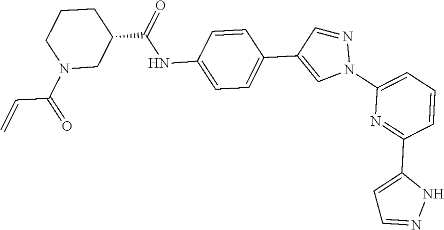 [SMILES: C=CC(=O)N1CCC[C@H](C(=O)Nc2ccc(-c3cnn(-c4cccc(-c5ccn[nH]5)n4)c3)cc2)C1] |
| 073 | 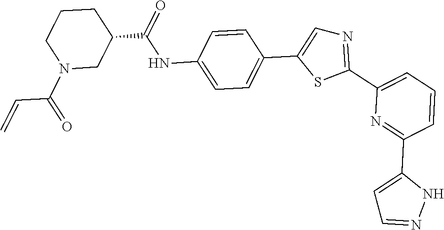 [SMILES: C=CC(=O)N1CCC[C@H](C(=O)Nc2ccc(-c3cnc(-c4cccc(-c5ccn[nH]5)n4)s3)cc2)C1] |
| 074 | 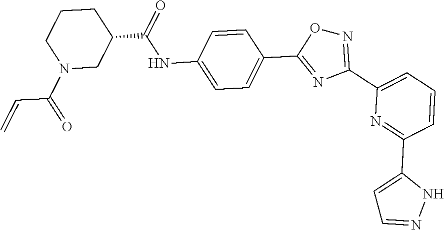 [SMILES: C=CC(=O)N1CCC[C@H](C(=O)Nc2ccc(-c3nc(-c4cccc(-c5ccn[nH]5)n4)no3)cc2)C1] |

or a pharmaceutically acceptable salt thereof.

In still another aspect, provided herein is a compound of Formula VI:

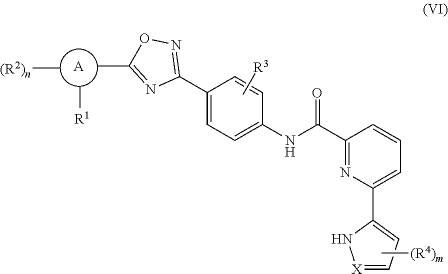

or a pharmaceutically acceptable salt thereof;
   wherein
X is N, CH, or CR⁴;
A is 6-7 membered heterocycloalkyl;
each R² is independently selected from the group consisting of halo, C₁-C₆ alkyl, and C₁-C₆ haloalkyl;
R³ is selected from the group consisting of H, halo, C₁-C₆ haloalkyl, C₁-C₆ alkoxy, and C₁-C₆ haloalkoxy;
each R⁴ is independently selected from the group consisting of C₁-C₆ alkyl, halo, and CN;
m is 0, 1, or 2;
n is 0, 1, or 2;
R¹ is selected from the group consisting of:

[chemical structure]

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, cis-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl; R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7}; and
R_{E7} is hydrogen or C₁-C₆ alkyl.

In an embodiment, X is N. In an embodiment, X is CR⁴.

In another embodiment, R³ is H.

In yet another embodiment, n is 0. In still another embodiment, n is 1, In an embodiment, n is 2.

In another embodiment, m is 0. In yet another embodiment, m is 1, In still another embodiment, m is 2.

In an embodiment, R¹ is

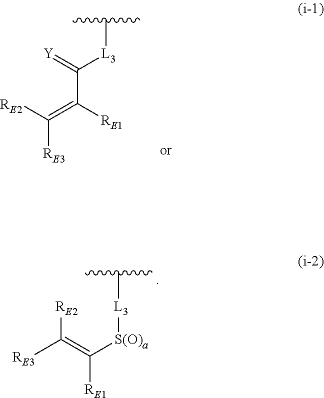

In another embodiment, R¹ is

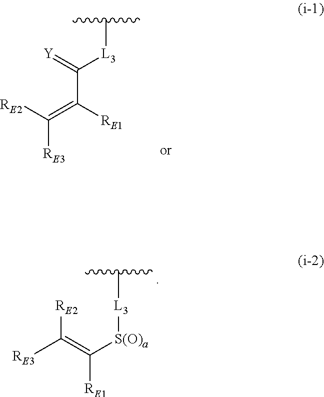

or
wherein
L₃ is —NH— or a bond;
Y is O;
each of R_{E1}, R_{E2}, and R_{E3} is independently selected from the group consisting of hydrogen and halogen; and
a is 2.

In yet another embodiment, R¹ is selected from the group consisting of

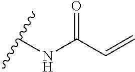 [SMILES: *NC(=O)C=C]

In another embodiment, the compound of Formula VI is a compound of Formula VIa:

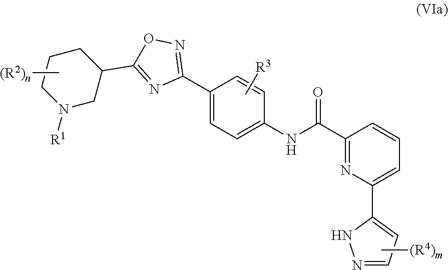

or a pharmaceutically acceptable salt thereof;

In yet another embodiment, the compound of Formula VI is a compound of Formula VIb:

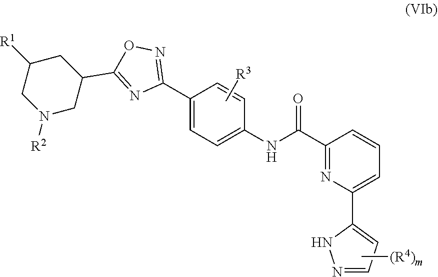

or a pharmaceutically acceptable salt thereof;

In still another embodiment, the compound of Formula VI is a compound of Formula VIc:

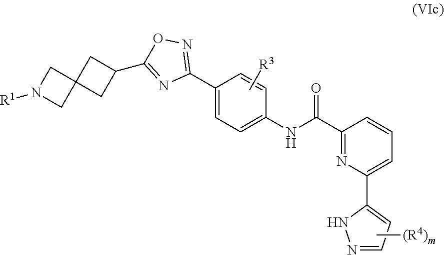

or a pharmaceutically acceptable salt thereof;

In another embodiment, the compound of Formula VI is selected from the group consisting of a compound in Table 7.

TABLE 7

| Compound No. | Structure |
|---|---|
| 075 | 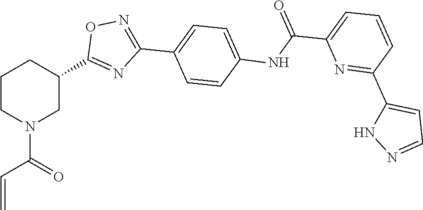 [SMILES: C=CC(=O)N1CCC[C@H](c2nc(-c3ccc(NC(=O)c4cccc(-c5ccn[nH]5)n4)cc3)no2)C1] |
| 076 | 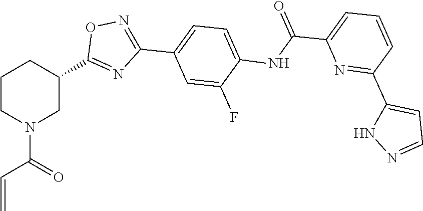 [SMILES: C=CC(=O)N1CCC[C@H](c2nc(-c3ccc(NC(=O)c4cccc(-c5ccn[nH]5)n4)c(F)c3)no2)C1] |
| 077 | 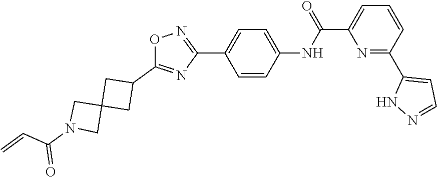 [SMILES: C=CC(=O)N1CC2(CC(c3nc(-c4ccc(NC(=O)c5cccc(-c6ccn[nH]6)n5)cc4)no3)C2)C1] |
| 078 | 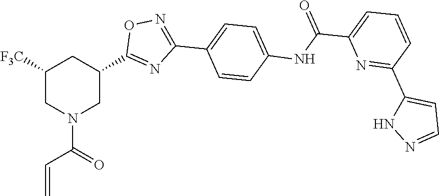 [SMILES: C=CC(=O)N1C[C@@H](c2nc(-c3ccc(NC(=O)c4cccc(-c5ccn[nH]5)n4)cc3)no2)C[C@@H](C(F)(F)F)C1] |
| 079 | 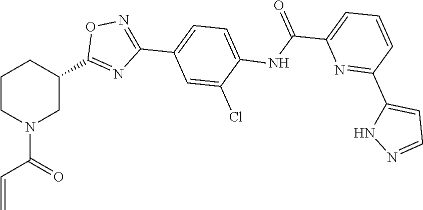 [SMILES: C=CC(=O)N1CCC[C@H](c2nc(-c3ccc(NC(=O)c4cccc(-c5ccn[nH]5)n4)c(Cl)c3)no2)C1] |
| 080 | 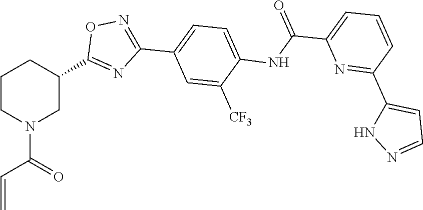 [SMILES: C=CC(=O)N1CCC[C@H](c2nc(-c3ccc(NC(=O)c4cccc(-c5ccn[nH]5)n4)c(C(F)(F)F)c3)no2)C1] |
| 081 | 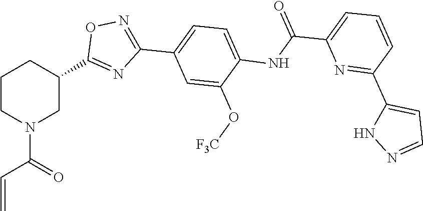 [SMILES: C=CC(=O)N1CCC[C@H](c2nc(-c3ccc(NC(=O)c4cccc(-c5ccn[nH]5)n4)c(OC(F)(F)F)c3)no2)C1] |
| 082 | 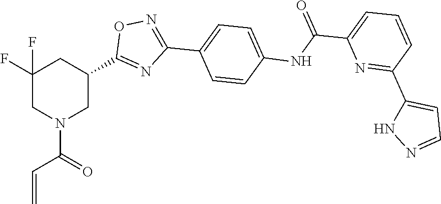 [SMILES: C=CC(=O)N1C[C@@H](c2nc(-c3ccc(NC(=O)c4cccc(-c5ccn[nH]5)n4)cc3)no2)CC(F)(F)C1] |
| 083 | 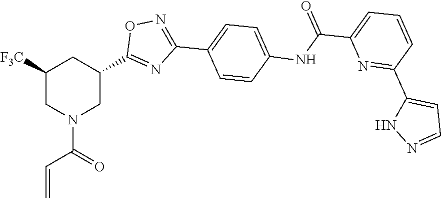 [SMILES: C=CC(=O)N1C[C@@H](c2nc(-c3ccc(NC(=O)c4cccc(-c5ccn[nH]5)n4)cc3)no2)C[C@H](C(F)(F)F)C1] |
| 084 | 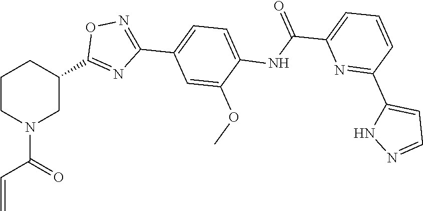 [SMILES: C=CC(=O)N1CCC[C@H](c2nc(-c3ccc(NC(=O)c4cccc(-c5ccn[nH]5)n4)c(OC)c3)no2)C1] |
| 085 | 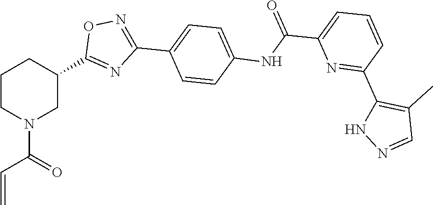 [SMILES: C=CC(=O)N1CCC[C@H](c2nc(-c3ccc(NC(=O)c4cccc(-c5[nH]ncc5C)n4)cc3)no2)C1] |
| 086 | 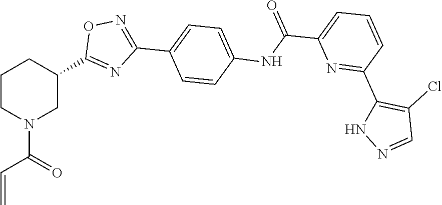 [SMILES: C=CC(=O)N1CCC[C@H](c2nc(-c3ccc(NC(=O)c4cccc(-c5[nH]ncc5Cl)n4)cc3)no2)C1] |
| 087 | 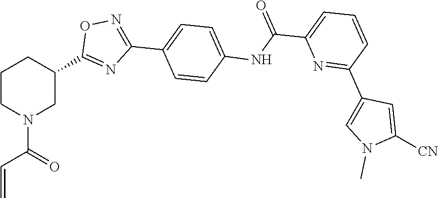 [SMILES: C=CC(=O)N1CCC[C@H](c2nc(-c3ccc(NC(=O)c4cccc(-c5cc(C#N)n(C)c5)n4)cc3)no2)C1] |
| 088 | 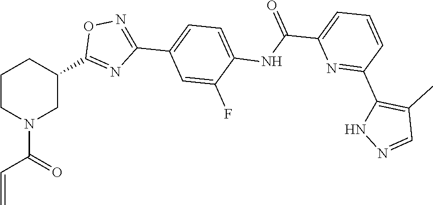 [SMILES: C=CC(=O)N1CCC[C@H](c2nc(-c3ccc(NC(=O)c4cccc(-c5[nH]ncc5C)n4)c(F)c3)no2)C1] |
| 089 | 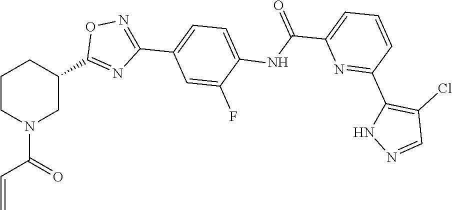 [SMILES: C=CC(=O)N1CCC[C@H](c2nc(-c3ccc(NC(=O)c4cccc(-c5[nH]ncc5Cl)n4)c(F)c3)no2)C1] |
| 090 | 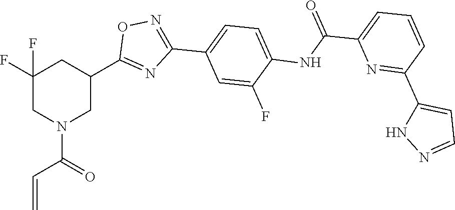 [SMILES: C=CC(=O)N1CC(c2nc(-c3ccc(NC(=O)c4cccc(-c5ccn[nH]5)n4)c(F)c3)no2)CC(F)(F)C1] |
| 091 | 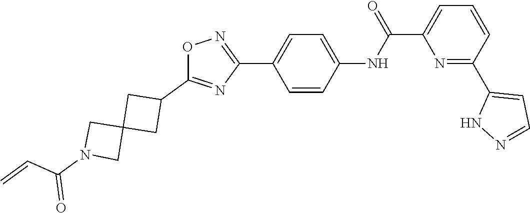 [SMILES: C=CC(=O)N1CC2(CC(c3nc(-c4ccc(NC(=O)c5cccc(-c6ccn[nH]6)n5)cc4)no3)C2)C1] |
| 092 | 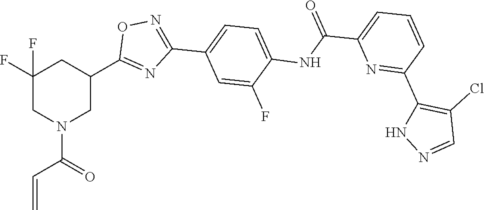 [SMILES: C=CC(=O)N1CC(c2nc(-c3ccc(NC(=O)c4cccc(-c5[nH]ncc5Cl)n4)c(F)c3)no2)CC(F)(F)C1] |
| 093 | 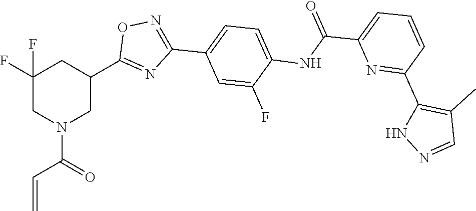 [SMILES: C=CC(=O)N1CC(c2nc(-c3ccc(NC(=O)c4cccc(-c5[nH]ncc5C)n4)c(F)c3)no2)CC(F)(F)C1] |
| 094 | 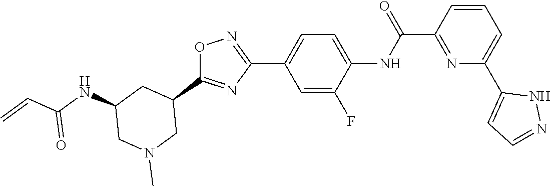 [SMILES: C=CC(=O)N[C@H]1C[C@@H](c2nc(-c3ccc(NC(=O)c4cccc(-c5ccn[nH]5)n4)c(F)c3)no2)CN(C)C1] |
| 095 | 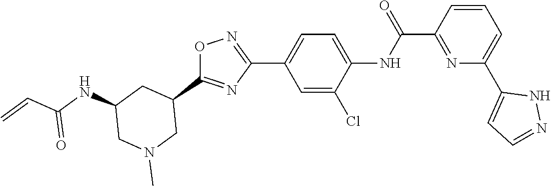 [SMILES: C=CC(=O)N[C@H]1C[C@@H](c2nc(-c3ccc(NC(=O)c4cccc(-c5ccn[nH]5)n4)c(Cl)c3)no2)CN(C)C1] |
| 096 | 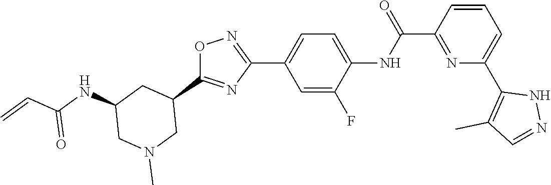 [SMILES: C=CC(=O)N[C@H]1C[C@@H](c2nc(-c3ccc(NC(=O)c4cccc(-c5[nH]ncc5C)n4)c(F)c3)no2)CN(C)C1] |
| 097 | 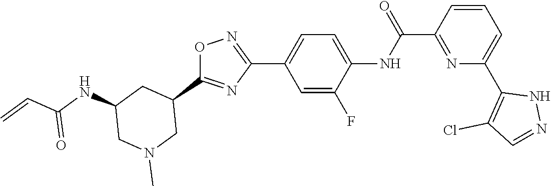 [SMILES: C=CC(=O)N[C@H]1C[C@@H](c2nc(-c3ccc(NC(=O)c4cccc(-c5[nH]ncc5Cl)n4)c(F)c3)no2)CN(C)C1] |
| 098 | 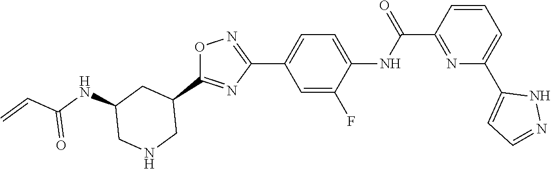 [SMILES: C=CC(=O)N[C@@H]1CNC[C@H](c2nc(-c3ccc(NC(=O)c4cccc(-c5ccn[nH]5)n4)c(F)c3)no2)C1] |

or a pharmaceutically acceptable salt thereof.

In another aspect, provided herein is a compound of Formula VII:

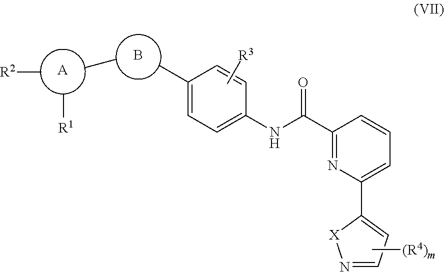

or a pharmaceutically acceptable salt thereof;
   wherein
X is NH or O;
A is 6-8 membered heterocycloalkyl or 05 cycloalkyl;
B is triazolyl;
R² is H or C₁-C₆ alkyl;
R³ is H or C₁-C₆ alkoxy;
R⁴ is halo;
m is 0 or 1;
R¹ is selected from the group consisting of:

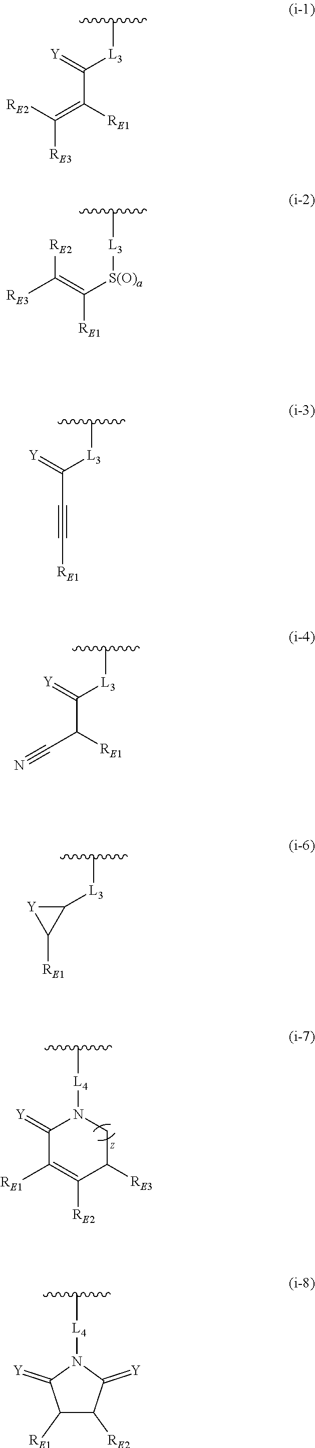

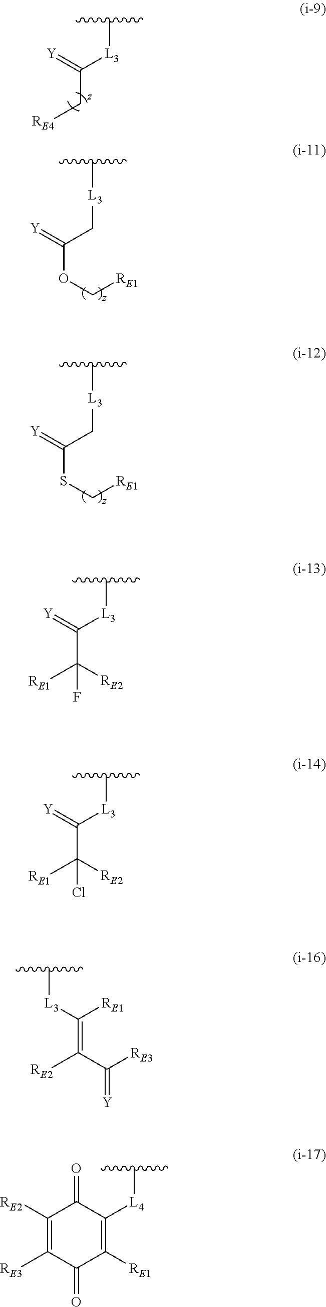

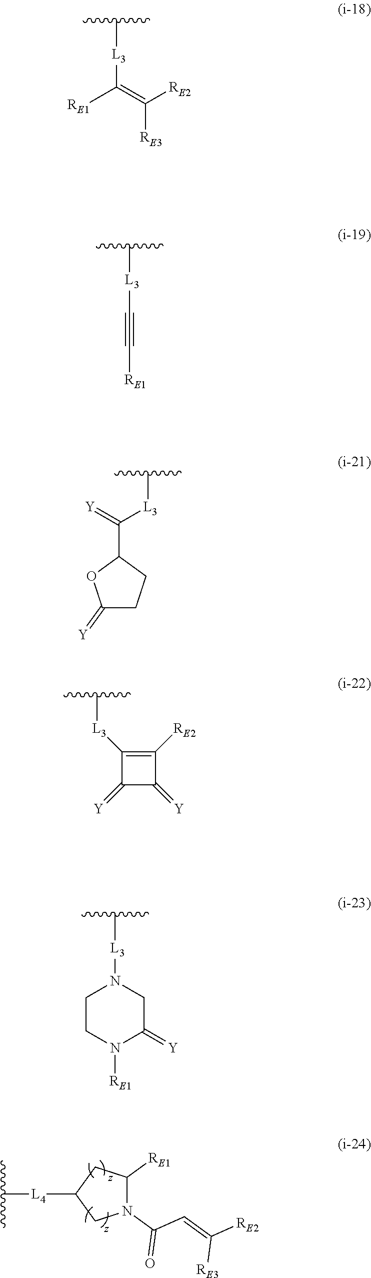

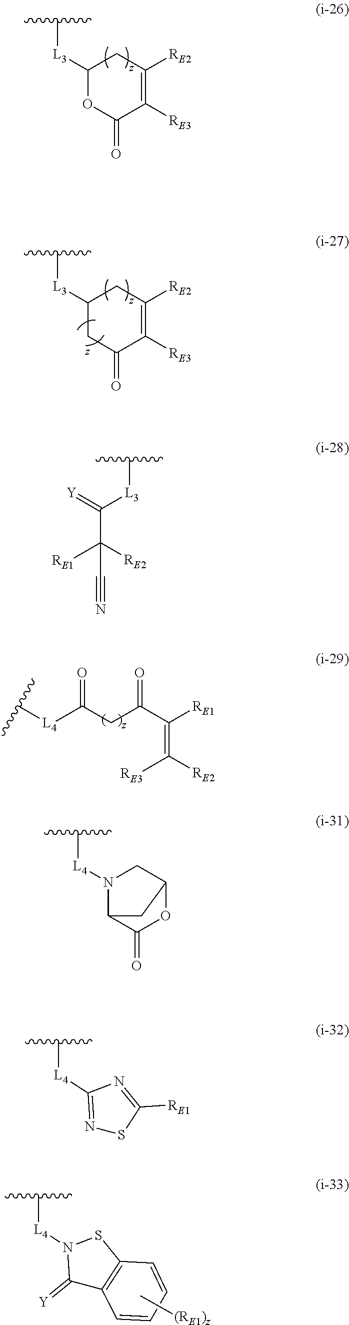

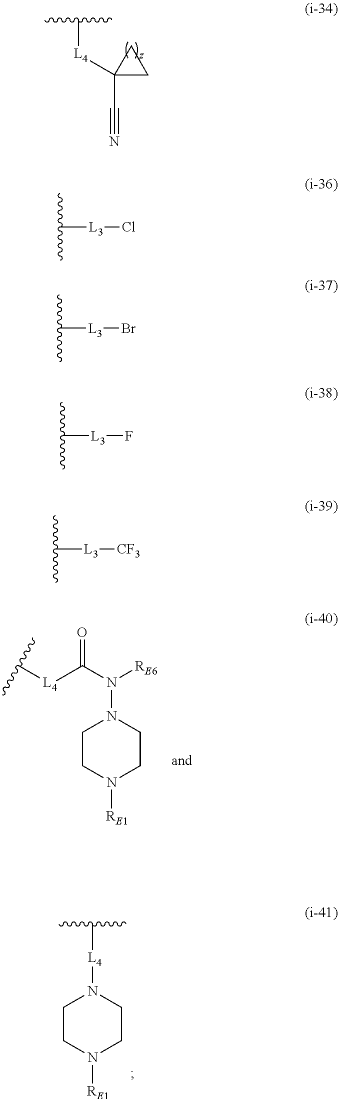

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, ciS-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7}; and
R_{E7} is hydrogen or C₁-C₆ alkyl.

In an embodiment, X is NH. In another embodiment, X is O.

In yet another embodiment, B is

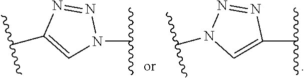

In still another embodiment, R² is H. In an embodiment, R² is C₁-C₆ alkyl. In another embodiment, R² is methyl.

In yet another embodiment, R³ is H. In still another embodiment, R³ is C₁-C₆ alkoxy. In an embodiment, R³ is methoxy.

In an embodiment, m is 0. In another embodiment, m is 1.

In yet another embodiment, the compound of Formula VII is a compound of Formula VIIa:

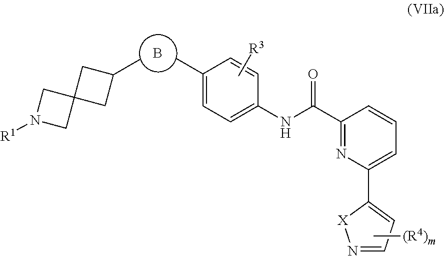

or a pharmaceutically acceptable salt thereof.

In still another embodiment, the compound of Formula VII is a compound of Formula VIIb:

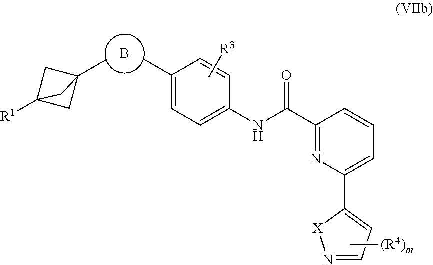

or a pharmaceutically acceptable salt thereof.

In another embodiment, the compound of Formula VII is a compound of Formula VIIc:

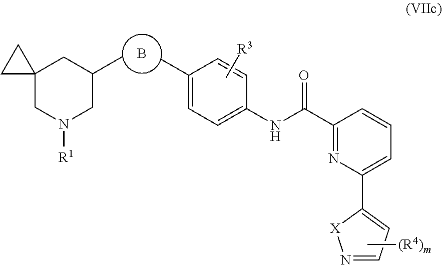

or a pharmaceutically acceptable salt thereof.

In yet another embodiment, the compound of Formula VII is a compound of Formula VIId:

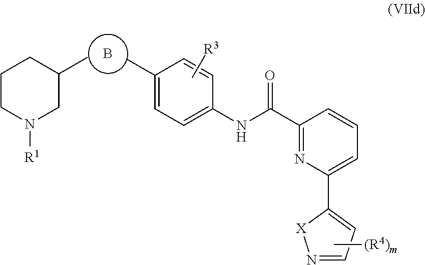

or a pharmaceutically acceptable salt thereof.

In still another embodiment, the compound of Formula VII is a compound of Formula

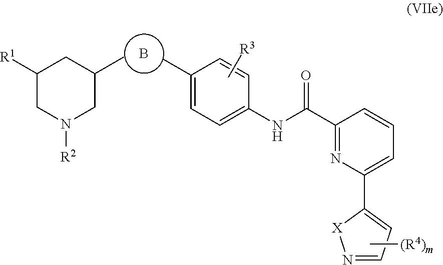

or a pharmaceutically acceptable salt thereof.

In an embodiment, R¹ is

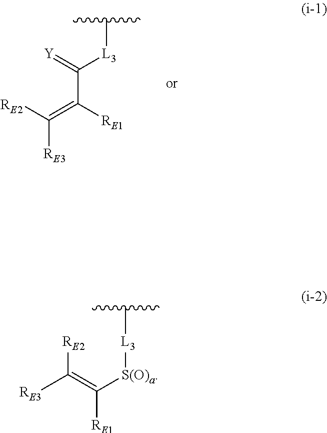

In another embodiment, R¹ is

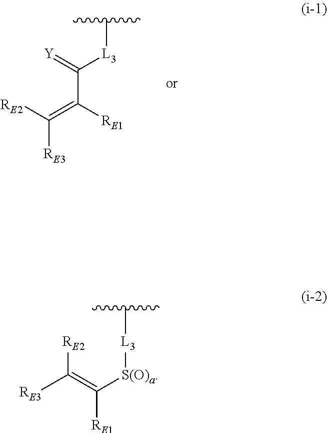

wherein
L₃ is —NH— or a bond;
Y is O;
each of R_{E}, R_{E2}, and R_{E3} is independently selected from the group consisting of hydrogen and halogen; and
a is 2.

In yet another embodiment, R¹ is selected from the group consisting of

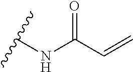 [SMILES: *NC(=O)C=C]

In another embodiment, the compound of Formula VII is selected from the group consisting of a compound in Table 8.

TABLE 8

| Compound No. | Structure |
|---|---|
| 099 | 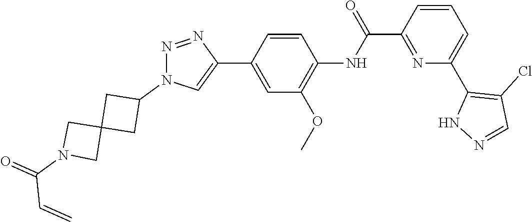 [SMILES: C=CC(=O)N1CC2(CC(n3cc(-c4ccc(NC(=O)c5cccc(-c6[nH]ncc6Cl)n5)c(OC)c4)nn3)C2)C1] |
| 100 | 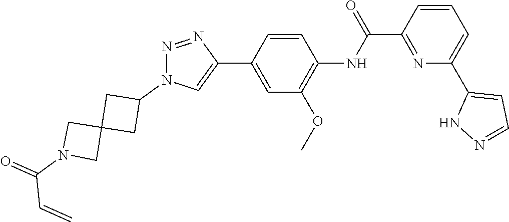 [SMILES: C=CC(=O)N1CC2(CC(n3cc(-c4ccc(NC(=O)c5cccc(-c6ccn[nH]6)n5)c(OC)c4)nn3)C2)C1] |
| 101 | 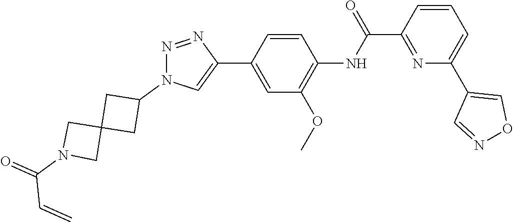 [SMILES: C=CC(=O)N1CC2(CC(n3cc(-c4ccc(NC(=O)c5cccc(-c6cnoc6)n5)c(OC)c4)nn3)C2)C1] |
| 102 | 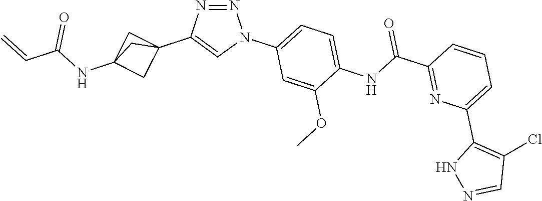 [SMILES: C=CC(=O)NC12CC(c3cn(-c4ccc(NC(=O)c5cccc(-c6[nH]ncc6Cl)n5)c(OC)c4)nn3)(C1)C2] |
| 103 | 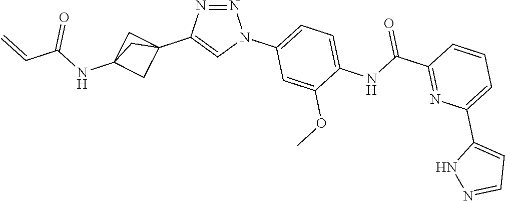 [SMILES: C=CC(=O)NC12CC(c3cn(-c4ccc(NC(=O)c5cccc(-c6ccn[nH]6)n5)c(OC)c4)nn3)(C1)C2] |
| 104 | 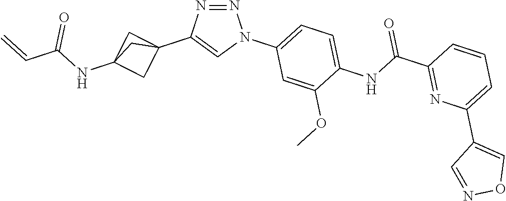 [SMILES: C=CC(=O)NC12CC(c3cn(-c4ccc(NC(=O)c5cccc(-c6cnoc6)n5)c(OC)c4)nn3)(C1)C2] |
| 105 | 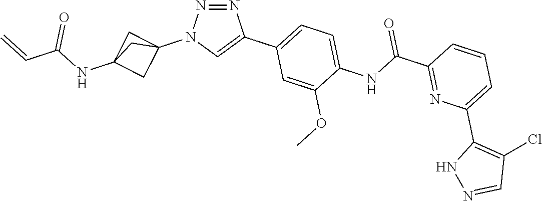 [SMILES: C=CC(=O)NC12CC(n3cc(-c4ccc(NC(=O)c5cccc(-c6[nH]ncc6Cl)n5)c(OC)c4)nn3)(C1)C2] |
| 106 | 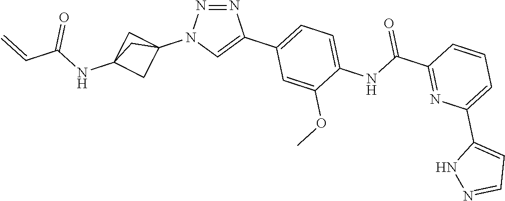 [SMILES: C=CC(=O)NC12CC(n3cc(-c4ccc(NC(=O)c5cccc(-c6ccn[nH]6)n5)c(OC)c4)nn3)(C1)C2] |
| 107 | 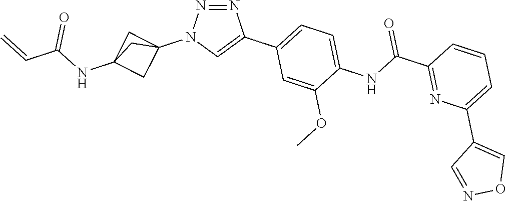 [SMILES: C=CC(=O)NC12CC(n3cc(-c4ccc(NC(=O)c5cccc(-c6cnoc6)n5)c(OC)c4)nn3)(C1)C2] |
| 108 | 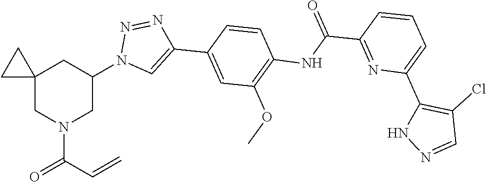 [SMILES: C=CC(=O)N1CC(n2cc(-c3ccc(NC(=O)c4cccc(-c5[nH]ncc5Cl)n4)c(OC)c3)nn2)CC2(CC2)C1] |
| 109 | 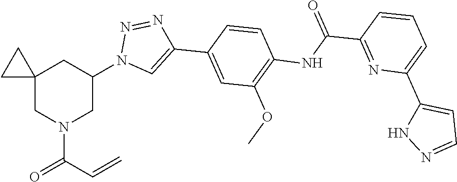 [SMILES: C=CC(=O)N1CC(n2cc(-c3ccc(NC(=O)c4cccc(-c5ccn[nH]5)n4)c(OC)c3)nn2)CC2(CC2)C1] |
| 110 | 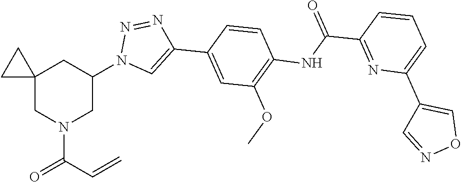 [SMILES: C=CC(=O)N1CC(n2cc(-c3ccc(NC(=O)c4cccc(-c5cnoc5)n4)c(OC)c3)nn2)CC2(CC2)C1] |
| 111 | 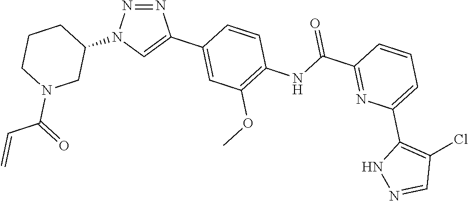 [SMILES: C=CC(=O)N1CCC[C@H](n2cc(-c3ccc(NC(=O)c4cccc(-c5[nH]ncc5Cl)n4)c(OC)c3)nn2)C1] |
| 112 | 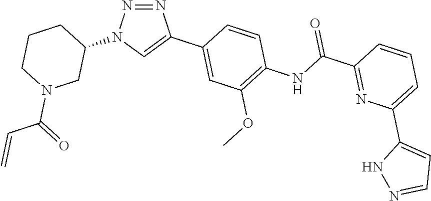 [SMILES: C=CC(=O)N1CCC[C@H](n2cc(-c3ccc(NC(=O)c4cccc(-c5ccn[nH]5)n4)c(OC)c3)nn2)C1] |
| 113 | 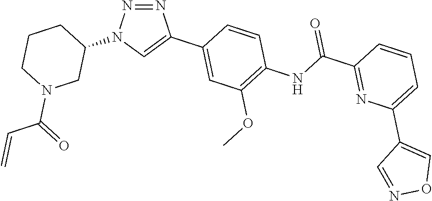 [SMILES: C=CC(=O)N1CCC[C@H](n2cc(-c3ccc(NC(=O)c4cccc(-c5cnoc5)n4)c(OC)c3)nn2)C1] |
| 114 | 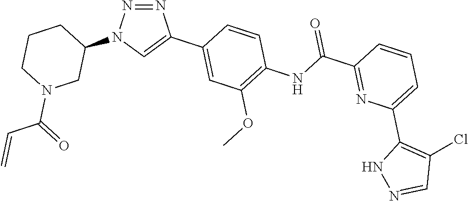 [SMILES: C=CC(=O)N1CCC[C@@H](n2cc(-c3ccc(NC(=O)c4cccc(-c5[nH]ncc5Cl)n4)c(OC)c3)nn2)C1] |
| 115 | 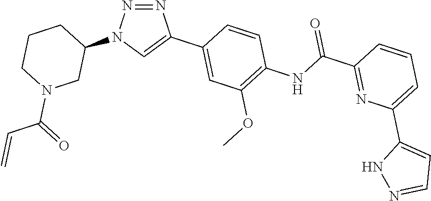 [SMILES: C=CC(=O)N1CCC[C@@H](n2cc(-c3ccc(NC(=O)c4cccc(-c5ccn[nH]5)n4)c(OC)c3)nn2)C1] |
| 116 | 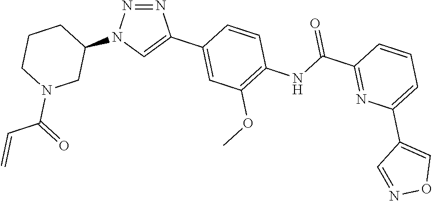 [SMILES: C=CC(=O)N1CCC[C@@H](n2cc(-c3ccc(NC(=O)c4cccc(-c5cnoc5)n4)c(OC)c3)nn2)C1] |
| 117 | 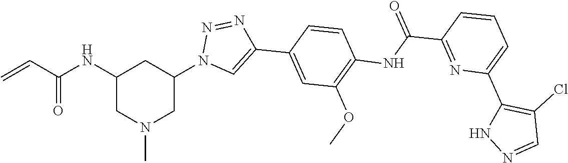 [SMILES: C=CC(=O)NC1CC(n2cc(-c3ccc(NC(=O)c4cccc(-c5[nH]ncc5Cl)n4)c(OC)c3)nn2)CN(C)C1] |
| 118 | 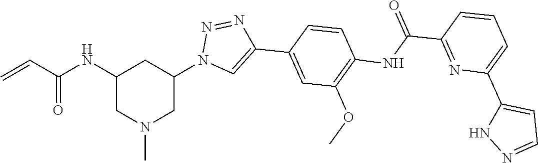 [SMILES: C=CC(=O)NC1CC(n2cc(-c3ccc(NC(=O)c4cccc(-c5ccn[nH]5)n4)c(OC)c3)nn2)CN(C)C1] |
| 119 | 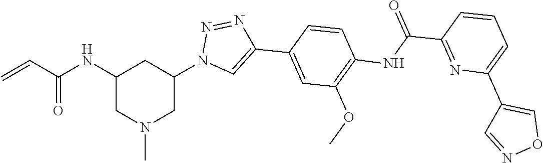 [SMILES: C=CC(=O)NC1CC(n2cc(-c3ccc(NC(=O)c4cccc(-c5cnoc5)n4)c(OC)c3)nn2)CN(C)C1] |

or a pharmaceutically acceptable salt thereof.

In an aspect, provided herein is a compound of any of the Formulae disclosed herein, or a pharmaceutically acceptable salt thereof, and a pharmaceutically acceptable carrier.

The compounds disclosed herein may exist as tautomers and optical isomers (e.g., enantiomers, diastereomers, diastereomeric mixtures, racemic mixtures, and the like).

In another aspect, the pharmaceutical composition further comprises a second active agent, wherein said second pharmaceutical agent. In some embodiments, the second pharmaceutical agent is a kinase inhibitor. In further embodiments, the second pharmaceutical agent is a Bruton's tyrosine kinase (BTK) inhibitor.

In yet another aspect, the present disclosure provides pharmaceutical compositions including a compound described herein, and optionally a pharmaceutically acceptable excipient.

In an embodiment, the pharmaceutical compositions described herein include a therapeutically or prophylactically effective amount of a compound described herein. The pharmaceutical composition may be useful for treating a proliferative disease in a subject in need thereof, preventing a proliferative disease in a subject in need thereof, or inhibiting the activity of a protein kinase (e.g., IRAK) in a subject, biological sample, tissue, or cell. In certain embodiments, the proliferative disease is cancer (e.g., lymphoma, leukemia, or myelodysplastic syndrome (MDS)). In certain embodiments, the proliferative disease is an inflammatory disease. In certain embodiments, the inflammatory disease is rheumatoid arthritis, Crohn' s disease, or fibrosis. In certain embodiments, the proliferative disease is an autoimmune disease.

### Methods of Treatment

In an aspect, provided herein is a method of treating cancer in an individual in need thereof, comprising administering to the individual a therapeutically effective amount of a compound of Formula I. In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another aspect, provided herein is a method of inhibiting a kinase in an individual in need thereof, comprising administering to the individual a therapeutically effective amount of a compound of Formula I. In an embodiment, the kinase is IRAK. In another embodiment, the kinase is IRAK1. In yet another embodiment, the kinase is IRAK4.

In another aspect, the present disclosure provides methods for treating and/or preventing a proliferative disease. Exemplary proliferative diseases that may be treated include diseases associated with the overexpression or increased activity of an interleukin-1 receptor-associated kinase (IRAK), e.g., cancer, benign neoplasms, diseases associated with angiogenesis, inflammatory diseases, autoinflammatory diseases, and autoimmune diseases. In certain embodiments, the cancer is selected from the group consisting of pancreatic cancer, lung cancer (e.g., small cell lung cancer (SCLC), non-small cell lung cancer), prostate cancer, breast cancer, ovarian cancer, kidney cancer, liver cancer, Ewing's sarcoma, myeloma, Waldenstrom's macroglobulinemia, myelodysplastic syndrome (MDS), osteosarcoma, brain cancer, neuroblastoma, and colorectal cancer.

In another aspect, provided herein is a method of inhibiting the activity of a kinase (e.g., IRAK (e.g., IRAK1 or IRAK4)) using a compound described herein in a biological sample or subject. In certain embodiments, the method involves the selective inhibition of IRAK1. In certain embodiments, the method involves the selective inhibition of IRAK4.

The present disclosure also provides methods of inhibiting cell growth in a biological sample or subject. In still another aspect, the present invention provides methods of inducing apoptosis of a cell in a biological sample or subject.

The present disclosure provides methods for administering to a subject in need thereof an effective amount of a compound, or pharmaceutical composition thereof, as described herein. Also described are methods for contacting a cell with an effective amount of a compound, or pharmaceutical composition thereof, as described herein. In certain embodiments, a method described herein further includes administering to the subject an additional pharmaceutical agent. In certain embodiments, a method described herein further includes contacting the cell with an additional pharmaceutical agent (e.g., an antiproliferative agent). In certain embodiments, the additional pharmaceutical agent is a kinase inhibitor (e.g., an inhibitor of Bruton's tyrosine kinase (BTK)). The methods described herein may further include performing radiotherapy, immunotherapy, and/or transplantation on the subject.

In yet another aspect, provided herein is a method of treating or preventing a kinase-mediated disorder in an individual in need thereof, comprising administering to the individual a therapeutically effective amount of a compound of Formula I.

Modulation of IRAK containing provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the compounds of the disclosure exhibit greater inhibition of IRAK1 relative to IRAK4. In certain embodiments, the compounds of the disclosure exhibit at least 2-fold, 3-fold, 5-fold, 10-fold, 25-fold, 50-fold or 100-fold greater inhibition of IRAK1 to IRAK4. In various embodiments, the compounds of the disclosure exhibit up to 1000-fold greater inhibition of IRAK1 relative IRAK4. In various embodiments, the compounds of the disclosure exhibit up to 10000-fold greater inhibition of IRAK1 relative to IRAK4.

In some embodiments, the inhibition of IRAK activity is measured by IC₅₀.

In some embodiments, the inhibition of IRAK activity is measured by EC₅₀.

In some embodiments, the inhibition of IRAK by a compound of the disclosure can be measured via a biochemical assay. By illustrative and non-limiting example, a homogenous time-resolved fluorescence (HTRF) assay may be used to determine inhibition of IRAK activity using conditions and experimental parameters disclosed herein. The HTRF assay may, for example, employ concentrations of substrate (e.g., biotin-Lck-peptide substrate) of about 1 μM; concentrations of IRAK from about 0.2 nM to about 40 nM; and concentrations of inhibitor from about 0,000282 μM to about 50 μM. A compound of the disclosure screened under these conditions may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

In some embodiments, the compounds of the disclosure bind irreversibly to IRAK.

Potency of the inhibitor can be determined by EC₅₀ value. A compound with a lower EC₅₀ value, as determined under substantially similar conditions, is a more potent inhibitor relative to a compound with a higher EC₅₀ value.

Potency of the inhibitor can also be determined by IC₅₀ value. A compound with a lower IC₅₀ value, as determined under substantially similar conditions, is a more potent inhibitor relative to a compound with a higher IC₅₀ value.

The selectivity between IRAK1 and IRAK4 can also be measured using cellular proliferation assays where cell proliferation is dependent on kinase activity. Proliferation assays are performed at a range of inhibitor concentrations (10 μM, 3 μM, 1.1 μM, 330 nM, 110 nM, 33 nM, 11 nM, 3 nM, 1 nM) and an EC₅₀ is calculated.

In still another aspect, the disclosure provides a method IRAK, the method comprising administering to a subject in need thereof an effective amount of a compound disclosed herein, or a pharmaceutically acceptable salt thereof. In some embodiments, the method further comprises administering a second pharmaceutical agent. In some embodiments, the second pharmaceutical agent is an antibody. In another embodiment, the second pharmaceutical agent is a kinase inhibitor. In yet another embodiment, the second pharmaceutical agent is a Bruton's tyrosine kinase (BTK) inhibitor.

The additional pharmaceutical agents include, but are not limited to, antiproliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent).

In certain embodiments, the additional pharmaceutical agent is ibrutinib. In certain embodiments, the additional pharmaceutical agent is a protein kinase inhibitor (e.g., tyrosine protein kinase inhibitor). In certain embodiments, the additional pharmaceutical agent is a binder or inhibitor of an IRAK (e.g., IRAK1 or IRAK4). In certain embodiments, the additional pharmaceutical agent is a binder or inhibitor of IRAK1. In certain embodiments, the additional pharmaceutical agent is a binder or inhibitor of IRAK4. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In yet another aspect, provided herein is a method of treating a kinase-mediated disorder comprising administering to a subject in need thereof an effective amount of a compound disclosed herein, or a pharmaceutically acceptable salt thereof. In some embodiments, the kinase is IRAK. In other embodiments, the subject is administered an additional therapeutic agent. In other embodiments, the compound and the additional therapeutic agent are administered simultaneously or sequentially.

In other embodiments, the disease is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which IRAK plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

In certain embodiments, the proliferative disease to be treated or prevented using the compounds described herein may be associated with the overexpression of an IRAK (e.g., IRAK1 or IRAK4).

A proliferative disease may be associated with aberrant activity of an IRAK (e.g., IRAK1 or IRAK4). Aberrant activity of an IRAK (e.g., IRAK1 or IRAK4) may be elevated and/or inappropriate or undesired activity of the IRAK. Deregulation of cell cycle progression is a characteristic of a proliferative disease, and a majority of proliferative diseases have abnormalities in some component of IRAK (e.g., IRAK1 or IRAK4) activity, frequently through elevated and/or inappropriate IRAK activation. In certain embodiments, IRAK is not overexpressed, and the activity of IRAK is elevated and/or inappropriate. In certain embodiments, IRAK1 is overexpressed, and the activity of IRAK1 is elevated and/or inappropriate. In certain embodiments, IRAK4 is overexpressed, and the activity of IRAK4 is elevated and/or inappropriate.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colonrectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliterative disease, or a neurodegenerative disease, comprising administering an effective amount of a compound, or a pharmaceutically acceptable composition comprising a compound, to a subject in need thereof.

The activity of the compounds and compositions of the present disclosure as IRAK inhibitors may be assayed in vitro, in vivo, or in a cell line. In vitro assays include assays that determine inhibition of either the kinase activity or ATPase activity of the activated kinase. Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this disclosure as an inhibitor of various kinases are set forth in the Examples below.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent. For any of the above uses, the required dosage will vary depending on the mode of administration, the particular condition to be treated and the effect desired.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, a Bruton's tyrosine kinase (BTK) inhibitor, chemotherapeutic agents, or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer.

In another aspect, provided herein is a kit comprising a compound capable of inhibiting IRAK activity selected from a compound disclosed herein, or a pharmaceutically acceptable salt thereof.

In another aspect, the disclosure provides a kit comprising a compound capable of inhibiting kinase activity selected from one or more compounds of disclosed herein, or pharmaceutically acceptable salts thereof; a second active agent; and instructions for use in treating cancer. In some embodiments, the second active agent is a Bruton's tyrosine kinase (BTK) inhibitor. In an embodiment, the BTK inhibitor is ibrutinib. In another embodiment, the BTK inhibitor is acalabrutinib. In yet another embodiment, the BTK inhibitor is zanubrutinib.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### ABBREVIATIONS

DCE dichloroethane
DCM dichloromethane
DIAD diisopropyl azodicarboxylate
DIEA diisopropylethylamine
DMF dimethylformamide
DMSO dimethylsulfoxide
EDCI 1-ethyl-3-(3-dimethylaminopropyl)carbodiimide
Et₃N/TEA triethylamine
HATU 1-[bis(dimethylamino)methylene]-1H-1,2,3-triazolo[4,5-b]pyridinium 3-oxide hexafluorophosphate
HOBt hydroxybenzotriazole
MeOH methanol
NCS N-chlorosuccinimide
TFA trifluoroacetic acid
THE tetrahydrofuran
THP tetrahydropyran
TsCl tosyl chloride

### Example 1: Synthetic Protocols

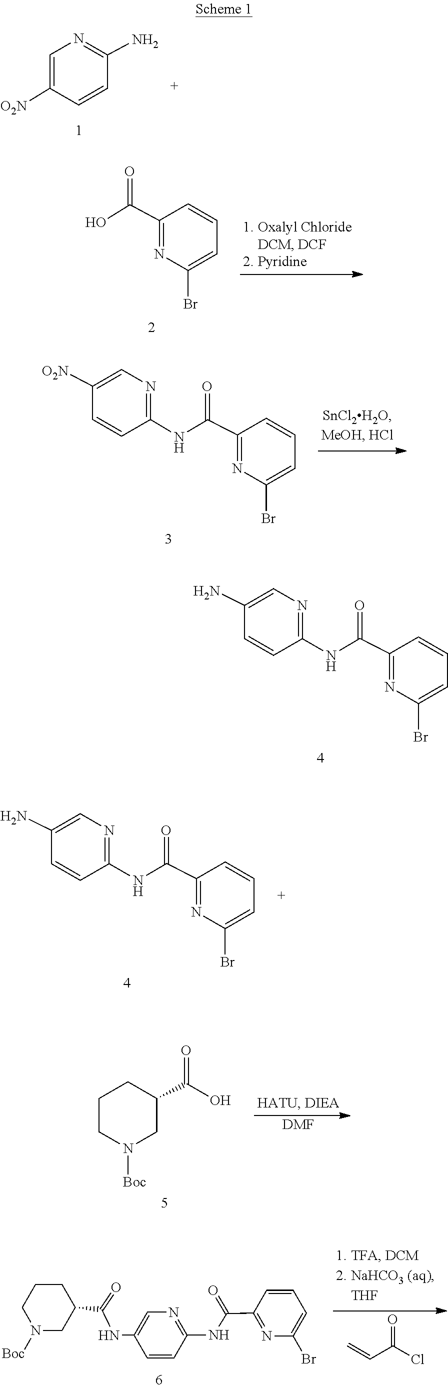

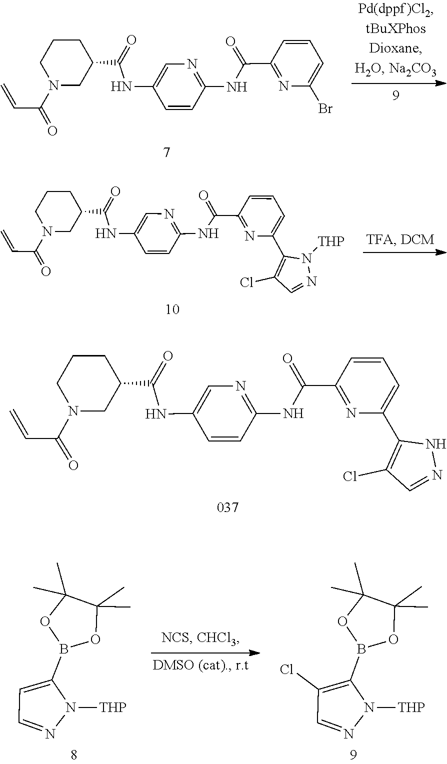

### 6-bromo-N-(5-nitropyridin-2-yl)picolinamide (3)

To a solution of 6-bromopicolinic acid (1 g, 4.95 mmol) in DCM (30 mL) was added Oxalyl Chloride (2.12 mL, 24.75 mmol) followed by DMF (5 drops). The mixture was stirred for 1 hour, then the solvent was removed. The residue was suspended in pyridine (10 mL) and a solution of 5-nitropyridin-2-amine (688 mg, 4.85 mmol) in Pyridine (10 mL) was added dropwise at 0°C. After addition was complete, the mixture was warmed to rt and stirred for 1 hour. The reaction was quenched with sat. aq. NaHCO₃ and extracted with DCM, dried over MgSO₄ and condensed. The resulting brown residue was triturated with EtOAc to yield a brown precipitate which was filtered, dried under N₂ and used in the next step without further purification. m/z ESI expected: 323.11, observed: 324.72

### N-(5-aminopyridin-2-yl)-6-bromopicolinamide (4)

To a solution of 6-bromo-N-(5-nitropyridin-2-yl)picolinamide (1.5 g, 4.64 mmol) in MeOH (30 mL) was added conc. HCl (5 mL) followed by SnCl₂·2H₂O (3.14 g, 13.93 mmol). The mixture was stirred at 60°C for 1 hour. The mixture was cooled to rt and diluted with EtOAc. NH₄OH was added dropwise to adjust the pH to 6. Solid Na₂CO₃ was added to adjust pH to 10 following which MgSO₄ was added and stirred. The mixture was filtered and concentrated to yield the desired product as a brown solid that was used without further purification. m/z ESI expected: 293.12, observed: 294.64

### tert-butyl (S)-3-((6-(6-bromopicolinamido)pyridin-3-yl)carbamoyl)piperidine-1-carboxylate (6)

To a solution of N-(5-aminopyridin-2-yl)-6-bromopicolinamide (500 mg, 1.71 mmol), HATU (1.3 g, 3.42 mmol) and (S)-1-(tert-butoxycarbonyl)piperidine-3-carboxylic acid (430.2 mg, 1.87 mmol) in DMF (5 mL) was added DIEA (1.49 mL, 8.55 mmol). The mixture was stirred at rt until consumption of starting material and then purified by normal phase flash chromatography using a gradient of 10%-50% ethyl acetate in hexanes to give the desired compound as a solid (550 mg, 64% yield) m/z ESI expected: 504.39, observed (M+H⁺): 505.76

### (S)-N-(5-(1-acryloylpiperidine-3-carboxamido)pyridin-2-yl)-6-bromopicolinamide (7)

tert-butyl (S)-3-((6-(6-bromopicolinamido)pyridin-3-yl)carbamoyl)piperidine-1-carboxylate (100 mg, 0.19 mmol) was dissolved in dichloromethane (5 mL). TFA (1 mL) was added and the mixture was stirred for 60 minutes. The solvents were removed in vacuo and the crude was dissolved in THE (2 mL). To this was added sat. NaHCO₃(aq) (2 mL), acryloyl chloride (19.3 μL, 0.24 mmol), stirred at room temperature until consumption of starting material as monitored by LC/MS. The reaction was quenched by addition of water and extracted with EtOAC (2×10 mL) washed with brine, dried over MgSO₄ and condensed. The crude was carried forward without further purification. m/z expected: 458.32, observed (M+H⁺): 459.77

### 4-chloro-1-(tetrahydro-2H-pyran-2-yl)-5-(4,4,5,5-tetramethyl-1,3,2-dioxaborolan-2-yl)-1H-pyrazole (9)

To a solution of 1-(tetrahydro-2H-pyran-2-yl)-5-(4,4,5,5-tetramethyl-1,3,2-dioxaborolan-2-yl)-1H-pyrazole (1 g, 3.59 mmol) in CHCl₃ (50 mL) was added N-chlorosuccinimide (579.5 mg, 4.31 mmol) followed by anhyd. DMSO (51 μL, 0.71 mmol) and stirred at room temperature overnight until completion of reaction. The solvents were removed in vacuo, the crude was dissolved in ethyl acetate, quenched with water, the aqueous layer extracted with EtOAC (2×50 mL). The combined organic layers were washed with brine, dried over MgSO₄ and condensed. The crude was purified by flash chromatography 5%-30% ethyl acetate in hexanes to give 700 mg of the desired product as an oil. m/z expected: 312.6, observed: 312.26, 228.17, 147.01

### (S)-N-(5-(1-acryloylpiperidine-3-carboxamido)pyridin-2-yl)-6-(4-chloro-1H-pyrazol-5-yl)picolinamide (Compound 037)

To a solution (S)-N-(5-(1-acryloylpiperidine-3-carboxamido)pyridin-2-yl)-6-bromopicolinamide (50 mg, 0.11 mmol), in 1,4-Dioxane (2 mL), was added 4-chloro-1-(tetrahydro-2H-pyran-2-yl)-5-(4,4,5,5-tetramethyl-1,3,2-dioxaborolan-2-yl)-1H-pyrazole (44.2 mg, 0.14 mmol) followed by 2M Na₂CO₃ (aq) (0.28 mL, 0.55 mmol). The mixture was degassed in a sonicator for 2 minutes. Pd(dppf)Cl₂ (9.66 mg, 0.013 mmol) and t-BuXPhos (8.41 mg, 0.019 mmol) were added and the mixture heated to 90°C in a sealed vial for 1 hour. The reaction was quenched with water (5 mL) and extracted with ethyl acetate (2×10 mL) washed with brine, dried over MgSO₄ and condensed. The crude material was dissolved in DCM (2 mL) and TFA (0.4 mL) was added. The mixture was stirred for 1 h and the solvent removed in vacuo. The crude material was purified by reversed phase HPLC to give the desired compound (4.9 mg, 31% yield). m/z expected: 479.53, observed: 480.31; ¹H NMR (500 MHz DMSO) δ 11.13 (s, 1H), 10.28 (d, J=14.9 Hz, 1H), 8.70 (s, 1H), 8.30-8.27 (m, 2H), 8.22 (t, J=7.7 Hz, 1H), 8.19-8.17 (m, 1H), 8.07 (t, J=8.8 Hz, 1H), 7.92 (s, 1H), 6.93-6.81 (m, 1H), 6.10 (dd, J=16.3, 2.03 Hz, 1H), 5.69-5.66 (m, 1H), 4.51-4.01 (m, 3H), 3.28-3.06 (m, 1H), 2.85-2.75 (m, 1H), 2.02-1.98 (m, 1H), 1.80-1.71 (m, 2H), 1.40-1.37 (m, 1H).

### tert-butyl 5-((6-(6-bromopicolinamido)pyridin-3-yl)carbamoyl)-3,3-difluoropiperidine-1-carboxylate (12)

The same procedure for 6 was followed with the solvent as 1,2,dichloroethane to give 630 mg of the desired product in 69% yield. m/z expected: 540.37, observed: 540.29; ¹H NMR (500 MHz DMSO) δ 10.47 (s, 1H), 10.14 (s, 1H), 8.63 (s, 1H), 8.21 (d, J=8.9 Hz, 1H), 8.19 (d, J=8.1 Hz, 1H), 8.09 (dd, J=8.9, 2.6 Hz, 1H), 8.05 (t, J=7.7 Hz, 1H), 7.97 (d, J=7.8 Hz, 1H), 4.14 (s, 2H), 3.75 (s, 2H), 2.86-2.80 (m, 1H), 2.41 (s, 1H), 2.26-2.13 (m, 1H), 1.43 (s, 9H).

### N-(5-(1-acryloyl-5,5-difluoropiperidine-3-carboxamido)pyridin-2-yl)-6-(1H-pyrazol-5-yl)picolinamide (Compound 038)

To a solution of tert-butyl 5-((6-(6-bromopicolinamido)pyridin-3-yl)carbamoyl)-3,3-difluoropiperidine-1-carboxylate (50 mg, 0.09 mmol), in 1,4-Dioxane (2 mL), was added 1-(tetrahydro-2H-pyran-2-yl)-5-(4,4,5,5-tetramethyl-1,3,2-dioxaborolan-2-yl)-1H-pyrazole (33.46 mg, 0.12 mmol) followed by 2M Na₂CO₃(aq) (0.23 mL, 0.46 mmol). The mixture was degassed in a sonicator for 2 minutes. Pd(dppf)Cl₂ (8.08 mg, 0.011 mmol) and t-BuXPhos (7.03 mg, 0.017 mmol) were added, and the mixture heated to 90°C in a sealed vial for 1 hour. The reaction was quenched with water (5 mL) and extracted with ethyl acetate (2×10 mL) washed with brine, dried over MgSO₄ and concentrated in vacuo. The crude was dissolved in dichloromethane (5 mL). TFA (1 mL) was added, and the mixture was stirred for 60 minutes. The solvents were removed in vacuo and the crude was dissolved in THE (2 mL), sat. NaHCO₃ (aq) (2 mL), acryloyl chloride (6.3 μL, 0.074 mmol) were added, stirred at room temperature until consumption of starting material as monitored by LC/MS. The reaction was quenched by addition of water and extracted with ethyl acetate (2×10 mL) washed with brine, dried over MgSO₄ and condensed. The crude was purified by reversed phase HPLC to give the desired compound (17.7 mg, 59% yield). m/z expected: 481.46, observed: 482.36; ¹H NMR (500 MHz DMSO) δ 11.05 (s, 1H), 10.47 (d, J=22.2 Hz, 1H), 8.72 (s, 1H), 8.28 (d, J=8.2 Hz, 1H), 8.16-8.14 (m, 1H), 8.13-8.10 (m, 2H), 8.09 (dd, J=9.1, 2.7 Hz, 1H), 7.74 (s, 1H), 7.06 (s, 1H) 7.02-6.87 (m, 1H), 6.20 (d, J=17.4 Hz, 1H), 5.79-5.76 (m, 1H), 4.68-4.63 (m, 1H), 4.52-4.31 (m, 2H), 3.70-3.61 (m, 1H), 2.93-2.81 (m, 2H), 2.36-2.23 (m, 1H).

### N-(5-(1-acryloyl-4,4-difluoropiperidine-3-carboxamido)pyridin-2-yl)-6-(1H-pyrazol-5-yl)picolinamide (Compound 039)

Prepared according to the procedure for 038 to give 7.9 mg of the desired product in 48% yield. m/z expected: 481.46, observed: 482.3; ¹H NMR (500 MHz DMSO) δ 11.12 (s, 1H), 10.45 (s, 1H), 8.68 (s, 1H), 8.28 (d, J=8.9 Hz, 1H), 8.15-8.13 (m, 2H), 8.12-8.10 (m, 1H), 8.03 (dd, J=8.9, 2.5 Hz, 1H), 7.73 (s, 1H), 7.06 (s, 1H), 6.91-6.81 (m, 1H), 6.15-6.09 (m, 1H), 5.74-5.63 (m, 1H), 3.98 (s, 1H), 3.93 (s, 1H), 3.75 (s, 2H), 3.68 (s, 1H), 2.36-2.30 (m, 1H), 1.30-1.24 (m, 1H).

### tert-butyl 3-((6-(6-bromopicolinamido)pyridin-3-yl)carbamoyl)-5-(trifluoromethyl)piperidine-1-carboxylate (15)

To a suspension of 1-pivaloyl-5-(trifluoromethyl)piperidine-3-carboxylic acid (100 mg, 0.34 mmol) in THE at 0°C, was added EDCI (158.34 mg, 1.02 mmol) and HOBt (137.82 mg, 1.02 mmol). This was stirred for 10 minutes following which N-(5-aminopyridin-2-yl)-6-bromopicolinamide (99.66 mg, 0.34 mmol) was added and reaction was stirred overnight at room temperature. The reaction does not go to completion and was quenched with water (5 mL) and extracted with ethyl acetate (2×10 mL) washed with brine, dried over MgSO₄ and concentrated in vacuo. The crude was purified by flash chromatography to yield 29.1 mg of the product in 43% yield. m/z expected: 572.38, observed: 572.27; ¹H NMR (500 MHz DMSO) δ 10.39 (s, 1H), 10.15 (s, 1H), 8.68-8.62 (m, 1H), 8.21-8.18 (m, 2H), 8.04 (t, J=7.6 Hz, 1H), 7.97 (dd, J=7.9, 0.9 Hz, 1H), 3.81-3.77 (m, 2H), 3.43-3.34 (m, 2H), 3.06 (s, 1H), 2.88 (s, 1H), 2.13 (s, 1H), 2.08 (s, 1H), 1.78 (s, 1H), 1.38-1.2 (m, 9H)

### N-(5-(1-acryloyl-5-(trifluoromethyl)piperidine-3-carboxamido)pyridin-2-yl)-6-(1H-pyrazol-5-yl)picolinamide (Compound 040)

Prepared according to the procedure for 038 to give 8.5 mg of the desired product in 33% yield. m/z expected: 513.48, observed: 514.32; ¹H NMR (500 MHz DMSO) δ 11.07 (s, 1H), 10.35 (s, 1H), 8.66 (s, 1H), 8.26 (d, J=8.9 Hz, 1H), 8.15-8.13 (m, 2H), 8.12-8.09 (m, 1H), 8.05-8.01 (m, 1H), 7.74 (s, 1H), 7.06 (s, 1H), 6.82-6.75 (m, 1H), 6.11-6.03 (m, 2H), 5.60-5.58 (m, 1H), 3.88-3.75 (m, 1H), 3.48-3.44 (m, 1H), 2.94 (s, 2H), 2.88 (s, 1H), 2.21-2.18 (m, 1H), 1.96-1.92 (m, 1H).

### N-(5-(1-acryloyl-5,5-difluoropiperidine-3-carboxamido)pyridin-2-yl)-6-(4-chloro-1H-pyrazol-5-yl)picolinamide (Compound 041)

Prepared according to the procedure for 038 to give 5.5 mg of the desired product in 11% yield. m/z expected: 600.02, observed: 600.38; ¹H NMR (500 MHz DMSO) δ 11.05 (s, 1H), 10.43 (d, J=Hz, 1H), 8.70 (s, 1H), 8.29 (d, J=8.9 Hz, 1H), 8.24-8.18 (m, 2H), 8.08 (dd, J=8.9, 2.3 Hz, 1H), 7.92 (s, 1H), 7.00-6.87 (m, 1H), 6.22-6.18 (m, 1H), 5.79-5.75 (m, 1H), 4.67-4.62 (m, 1H), 3.70-3.61 (m, 1H), 3.32-3.20 (m, 2H), 2.94-2.78 (m, 2H), 2.33-2.24 (m, 1H).

### N-(5-(1-acryloyl-5-(trifluoromethyl)piperidine-3-carboxamido)pyridin-2-yl)-6-(4-chloro-1H-pyrazol-5-yl)picolinamide (Compound 042)

Prepared according to the procedure for 038 to give 5.7 mg of the desired product in 11% yield. m/z expected: 547.92, observed: 548.05; ¹H NMR (500 MHz DMSO) δ 11.04 (s, 1H), 10.35 (d, J=16.3 Hz, 1H), 8.65 (s, 1H), 8.30-8.26 (m, 2H), 8.23-8.18 (m, 3H), 8.04-8.00 (m, 1H), 6.81-6.74 (m, 1H), 6.11-6.03 (m, 1H), 5.60-5.58 (m, 1H), 3.47-3.43 (m, 1H), 2.93 (s, 2H), 2.86 (s, 1H), 2.21-2.18 (m, 2H), 2.00-1.96 (m, 2H).

### N-(5-(1-acryloyl-4,4-difluoropiperidine-3-carboxamido)pyridin-2-yl)-6-(4-chloro-1H-pyrazol-5-yl)picolinamide (Compound 043)

Prepared according to the procedure for 038 to give 8.4 mg of the desired product in 15% yield. m/z expected: 515.91, observed: 516.06; ¹H NMR (500 MHz DMSO) δ 11.04 (s, 1H), 10.46 (s, 1H), 8.66 (s, 1H), 8.29 (d, J=8.9 Hz, 2H), 8.22 (t, J=7.6 Hz, 1H), 8.19 (d, J=7.5 Hz, 1H), 8.03 (dd, J=8.7, 1.5 Hz, 1H), 7.93 (s, 1H), 6.91-6.81 (m, 1H), 6.14-6.09 (m, 1H), 5.75-5.63 (m, 1H), 3.97 (s, 2H), 3.93 (s, 1H), 3.74 (s, 2H), 3.68 (s, 1H), 3.15 (s, 1H), 2.54 (s, 1H).

### (S)-6-bromo-N-(5-(1-(2-fluoroacryloyl)piperidine-3-carboxamido)pyridin-2-yl)picolinamide (20)

tert-butyl (S)-3-((6-(6-bromopicolinamido)pyridin-3-yl)carbamoyl)piperidine-1-carboxylate (85 mg, 0.17 mmol) was dissolved in dichloromethane (5 mL). TFA (1 mL) was added and the mixture was stirred for 60 minutes. The solvents were removed in vacuo and the crude was dissolved in DMF (2 mL). 2-fluoroacrylic acid (15.17 mg, 0.17 mmol), HATU (129.27 mg, 0.34 mmol) were added and stirred. DIEA (0.15 mL, 0.85 mmol) was then added and stirred at rt until completion. The reaction was quenched with water (5 mL) and extracted with ethyl acetate (2×10 mL) washed with brine, dried over MgSO₄ and concentrated in vacuo. The crude was purified by flash chromatography to give 55.7 mg of the product in 69% yield. m/z expected: 476.31, observed: 476.32; ¹H NMR (500 MHz DMSO) δ 10.27 (s, 1H), 10.13 (s, 1H), 8.62 (s, 1H), 8.21-8.16 (m, 2H), 8.09 (dd, J=8.9, 2.5 Hz, 1H), 8.05 (t, J=7.6 Hz, 1H), 7.97 (dd, J=8.0, 0.8 Hz, 1H), 5.27 (dd, J=18.0, 4.0 Hz, 1H), 5.21 (d, J=4.0 Hz, 0.5H), 5.11 (d, J=4.0 Hz, 0.5H), 4.35 (s, 1H), 4.12-3.85 (m, 2H), 3.18 (s, 1H), 2.95 (s, 1H), 2.03 (d, J=11.9 Hz, 1H), 1.83-1.71 (m, 2H), 1.47-1.40 (m, 1H).

### (S)-N-(5-(1-(2-fluoroacryloyl)piperidine-3-carboxamido)pyridin-2-yl)-6-(1H-pyrazol-5-yl)picolinamide (Compound 044)

Prepared according to the procedure for 038 to give 5.0 mg of the desired product in 8% yield. m/z expected: 547.59, observed: 548.26; ¹H NMR (500 MHz DMSO) δ 11.05 (s, 1H), 10.27 (s, 1H), 8.71 (s, 1H), 8.26 (d, J=8.9 Hz, 1H), 8.25, 8.15-8.14 (m, 1H), 8.12 (s, 1H), 8.11-8.09 (m, 1H), 8.06 (dd, J=8.9, 2.5 Hz, 1H), 7.74 (s, 1H), 7.06 (s, 1H), 5.21 (dd, J=18.2, 4.0 Hz, 1H), 5.11 (d, J=4.0 Hz, 0.5H), 5.11 (d, J=4.0 Hz, 0.5H), 4.38 (s, 1H), 3.17 (s, 1H), 2.93 (s, 1H), 2.05-2.03 (m, 1H), 1.83-1.79 (m, 1H), 1.77-1.74 (m, 1H), 1.48-1.41 (m, 1H).

### 6-bromo-N-(5-(5,5-difluoro-1-(2-fluoroacryloyl)piperidine-3-carboxamido)pyridin-2-yl)picolinamide (22)

Prepared according to the procedure for 20 to give 60.0 mg of the desired product in 64% yield. m/z expected: 512.29, observed: 512.64; ¹H NMR (500 MHz DMSO) δ 10.47 (s, 1H), 10.15 (s, 1H), 8.62 (s, 1H), 8.22 (d, J=8.9 Hz, 1H), 8.19 (dd, J=7.5, 0.9 Hz, 1H), 8.09 (dd, J=8.9, 2.5 Hz, 1H), 8.05 (t, J=7.6 Hz, 1H), 7.97 (dd, J=7.9, 0.9 Hz, 1H), 5.40 (dd, J=17.9, 4.5 Hz, 1H), 5.35 (d, J=4.2 Hz, 0.5H), 5.11 (d, J=4.1 Hz, 0.5H), 4.47 (s, 1H), 4.14 (s, 2H), 2.90 (s, 2H), 2.40-2.26 (m, 2H).

### N-(5-(5,5-difluoro-1-(2-fluoroacryloyl)piperidine-3-carboxamido)pyridin-2-yl)-6-(1H-pyrazol-5-yl)picolinamide (Compound 045)

Prepared according to the procedure for 038 to give 5.0 mg of the desired product in 10% yield. m/z expected: 499.45, observed: 500.41; 1H NMR (500 MHz DMSO) δ 11.07 (s, 1H), 10.47 (s, 1H), 8.70 (s, 1H), 8.28 (d, J=8.9 Hz, 1H), 8.14-8.11 (m, 3H), 8.07 (dd, J=8.9, 2.4 Hz, 1H), 7.74 (s, 1H), 7.06 (s, 1H), 5.41 (dd, J=17.9, 4.1 Hz, 1H), 5.34 (d, J=4.1 Hz, 0.5H), 5.24 (d, J=4.1 Hz, 0.5H), 4.41 (s, 1H), 4.08 (s, 1H), 2.90 (s, 2H), 2.41-2.26 (m, 3H).

### 6-bromo-N-(5-(5,5-difluoro-1-(vinylsulfonyl)piperidine-3-carboxamido)pyridin-2-yl)picolinamide (24)

Prepared according to the procedure for 20 to give 25.0 mg of the desired product in 51% yield. m/z expected: 512.29, observed: 512.64; ¹H NMR (500 MHz DMSO) δ 10.49 (s, 1H), 10.15 (s, 1H), 8.62 (s, 1H), 8.22 (d, J=8.9 Hz, 1H), 8.19 (dd, J=7.5, 0.9 Hz, 1H), 8.09-8.06 (m, 1H), 8.04 (d, J=7.7 Hz, 1H), 7.97 (dd, J=7.9, 0.9 Hz, 1H), 6.90 (d, J=10.0 Hz, 0.5H), 6.87 (d, J=10.0 Hz, 0.5H), 6.22 (s, 1H), 6.19 (d, J=8.2 Hz, 1H), 3.82-3.80 (m, 1H), 3.77-3.72 (m, 2H), 2.99-2.91 (m, 3H), 2.26-2.13 (m, 3H).

### N-(5-(5,5-difluoro-1-(vinylsulfonyl)piperidine-3-carboxamido)pyridin-2-yl)-6-(1H-pyrazol-5-yl)picolinamide (Compound 046)

Prepared according to the procedure for 038 to give 7.2 mg of the desired product in 29% yield. m/z expected: 517.51, observed: 518.43; 1H NMR (500 MHz DMSO) δ 11.34 (s, 1H), 10.47 (s, 1H), 8.70 (s, 1H), 8.27 (d, J=8.9 Hz, 1H), 8.14-8.09 (m, 3H), 8.06 (m, 1H), 7.74 (s, 1H), 7.06 (s, 1H), 6.90 (d, J=10.0 Hz, 0.5H), 6.87 (d, J=10.0 Hz, 0.5H), 6.53 (s, 1H), 6.22 (s, 1H), 6.19 (d, J=7.8 Hz, 1H), 3.82-3.80 (m, 1H), 3.77-3.72 (m, 1H), 3.00-2.91 (m, 3H), 2.27-2.15 (m, 2H).

### 6-(4-chloro-1H-pyrazol-5-yl)-N-(5-(5,5-difluoro-1-(2-fluoroacryloyl)piperidine-3-carboxamido)pyridin-2-yl)picolinamide (Compound 047)

To a solution of 6-bromo-N-(5-(5,5-difluoro-1-(2-fluoroacryloyl)piperidine-3-carboxamido)pyridin-2-yl)picolinamide (90 mg, 0.17 mmol), in 1,4-Dioxane (2 mL), was added 4-chloro-1-(tetrahydro-2H-pyran-2-yl)-5-(4,4,5,5-tetramethyl-1,3,2-dioxaborolan-2-yl)-1H-pyrazole (164.75 mg, 0.52 mmol) followed by 2M Na₂CO₃(aq) (0.44 mL, 0.92 mmol). The mixture was degassed in a sonicator for 2 minutes. PdAmphos (124.03 mg, 0.17 mmol) was added, and the mixture heated to 90°C in a sealed vial for 1 hour. The reaction was quenched with water (5 mL) and extracted with ethyl acetate (2×10 mL) washed with brine, dried over MgSO₄ and concentrated in vacuo. The crude was dissolved in dichloromethane (5 mL). TFA (1 mL) was added, and the mixture was stirred for 60 minutes. The solvents were removed in vacuo and the crude was purified by reversed phase HPLC to give the desired compound (20.0 mg, 22% yield). m/z expected: 533.12, observed: 533.9; ¹H NMR (500 MHz DMSO) δ 11.26 (s, 1H), 10.47 (s, 1H), 8.73 (s, 1H), 8.29 (d, J=8.9 Hz, 1H), 8.23-8.17 (m, 2H), 8.09 (d, J=7.8 Hz, 1H), 7.92 (s, 1H), 5.40 (dd, J=17.9, 4.1 Hz, 1H), 5.34 (d, J=4.1 Hz, 0.5H), 5.24 (d, J=4.1 Hz, 0.5H), 4.44 (s, 1H), 4.13 (s, 2H), 2.90 (s, 1H), 2.39-2.27 (m, 2H).

### 6-bromo-N-(5-(1-(2-fluoroacryloyl)-5-(trifluoromethyl)piperidine-3-carboxamido)pyridin-2-yl)picolinamide (27)

Prepared according to the procedure for 20 to give 100.0 mg of the desired product in 35% yield. m/z expected: 512.29, observed: 512.64; ¹H NMR (500 MHz DMSO) δ 10.41 (s, 1H), 10.36 (s, 1H), 8.58 (s, 1H), 8.28 (d, J=6.4 Hz, 1H), 8.23-8.18 (m, 2H), 8.03 (dd, J=7.3, 1.5 Hz, 1H), 7.67 (s, 1H), 6.94 (s, 1H), 6.07 (dd, J=9.7, 2.3 Hz, 1H), 3.98 (s, 1H), 3.65-3.61 (m, 1H), 2.92 (s, 1H), 2.43-2.36 (m, 1H), 2.21-2.16 (m, 1H), 2.08 (s, 1H), 1.91-1.90 (m, 1H), 1.74-1.67 (m, 1H), 1.56-1.46 (m, 1H).

### N-(5-(1-(2-fluoroacryloyl)-5-(trifluoromethyl)piperidine-3-carboxamido)pyridin-2-yl)-6-(1H-pyrazol-5-yl)picolinamide (Compound 048)

Prepared according to the procedure for 038 to give 30 mg in 31% yield. m/z expected: 531.47, observed: 532.5; ¹H NMR (500 MHz DMSO) δ 11.06 (s, 1H), 10.39 (s, 1H), 8.67 (s, 1H), 8.26 (d, J=8.9 Hz, 1H), 8.15-8.10 (m, 3H), 8.03 (dd, J=8.8, 1.9 Hz, 1H), 7.74 (s, 1H), 7.06 (s, 1H), 3.03 (s, 1H), 2.93 (s, 1H), 2.22-2.17 (m, 1H), 2.03-1.99 (m, 1H).

### 6-(4-chloro-1H-pyrazol-5-yl)-N-(5-(1-(2-fluoroacryloyl)-5-(trifluoromethyl)piperidine-3-carboxamido)pyridin-2-yl)picolinamide (Compound 049)

Prepared according to the procedure for 038 to give 2.5 mg in 2% yield. m/z expected: 565.91, observed: 566.49.

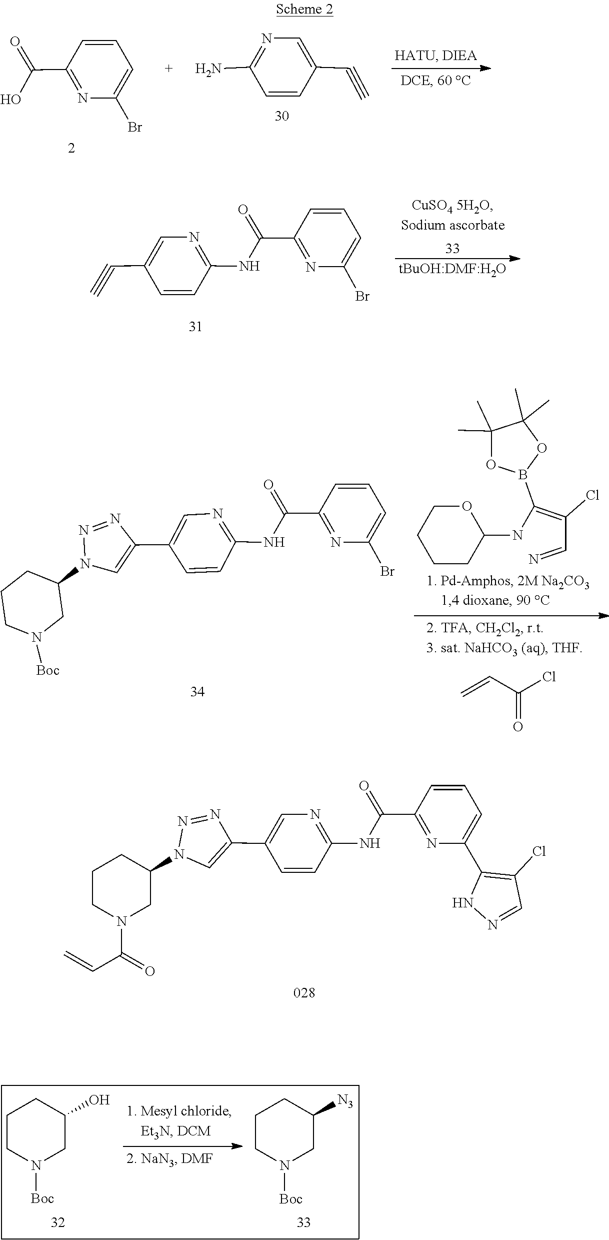

### 6-bromo-N-(5-ethynylpyridin-2-yl)picolinamide (31)

To a vial was added 5-ethynylpyridin-2-amine (900 mg, 7.62 mmol), 6-bromopicolinic acid (1.69 g, 8.38 mmol), HATU (5.79 g, 15.24 mmol) and 1,2 dichloroethane (10 mL). To the stirring reaction mixture at room temperature was added DIEA (6.63 mL, 38.1 mmol) dropwise and stirred at 60°C until completion of the reaction. The reaction was quenched with water, diluted with ethyl acetate. The aqueous layer was extracted with ethyl acetate (3×30 mL), combined, washed with brine, dried over MgSO₄, concentrated. The crude was purified by reverse phase chromatography (1-80% acetonitrile in water) to give 550 mg of the product in 23.7% yield. m/z ESI expected: 302.15, observed (M+H)⁺: 304.87. ¹H NMR (500 MHz DMSO-d6): δ 10.25 (s, 1H), 8.23 (d, J=9.6 Hz, 1H), 8.18 (dd, J=7.5 Hz, 1 Hz, 1H), 8.03 (dd, J=5.0, 10.0 Hz, 1H), 7.99 (dd, J=2.3, 8.6 Hz, 1H), 7.96 (dd, J=1.0, 7.9 Hz, 1H), 4.38 (s, 1H).

### tert-butyl (R)-3-azidopiperidine-1-carboxylate (33)

To a flask containing tert-butyl (S)-3-hydroxypiperidine-1-carboxylate (1 g, 4.96 mmol) in DCM (5 mL) was added TEA (1.38 mL, 9.92 mmol) at 0°C and mesyl chloride (0.46 mL, 5.96 mmol) dropwise and set to stir until completion of reaction. The solvents were evaporated in vacuo, the crude was dissolved in ethyl acetate and washed with NaHCO₃ (aq) (3×10 mL). The organic layers were washed with brine, dried over MgSO₄, concentrated. The crude was dissolved in DMF (2 mL) and NaN₃ (2.56 g, 39.68 mmol) was added and stirred at 100°C until consumption of the starting mesylate material. The reaction mixture was diluted with ethyl acetate, washed with NaHCO₃(aq) (3×10 mL). The organic layers were washed with brine, dried over MgSO₄, concentrated. The crude was purified by flash chromatography (2%-25% ethyl acetate in hexanes) to give 479.2 mg of the product in 49.7% yield. m/z expected: 226.28, observed: 170.91 (without the BOC group); ¹H NMR (500 MHz DMSO) δ 3.69 (s, 1H), 3.58-3.40 (m, 2H), 3.29-3.17 (m, 2H), 1.82 (s, 1H), 1.58 (s, 2H), 1.4 (s, 9H), 1.38-1.35 (m, 1H)

### tert-butyl (R)-3-(4-(6-(6-bromopicolinamido)pyridin-3-yl)-1H-1,2,3-triazol-1-yl)piperidine-1-carboxylate (34)

To a vial containing 6-bromo-N-(5-ethynylpyridin-2-yl)picolinamide (150 mg, 0.49 mmol) in 1:1:1 DMF: water: tert-butanol (3 mL) was added tert-butyl (R)-3-azidopiperidine-1-carboxylate (110.88 mg, 0.49 mmol), CuSO₄·5H₂O (125 mg, 0.49 mmol), sodium ascorbate (99.1 mg, 0.49 mmol) and stirred at 60°C until completion of reaction. The reaction mixture was quenched with water, filtered. The solid was dissolved in DMSO and purified by reverse phase HPLC to give 189.1 mg of product in 72.9% yield. m/z expected: 528.41, observed: 528.74

### (R)-N-(5-(1-(1-acryloylpiperidin-3-yl)-1H-1,2,3-triazol-4-yl)pyridin-2-yl)-6-(4-chloro-1H-pyrazol-5-yl)picolinamide (Compound 028)

To a solution of tert-butyl (R)-3-(4-(6-(6-bromopicolinamido)pyridin-3-yl)-1H-1,2,3-triazol-1-yl)piperidine-1-carboxylate (100 mg, 0.17 mmol), in 1,4-Dioxane (2 mL), was added 4-chloro-1-(tetrahydro-2H-pyran-2-yl)-5-(4,4,5,5-tetramethyl-1,3,2-dioxaborolan-2-yl)-1H-pyrazole (164.75 mg, 0.52 mmol) followed by 2M Na₂CO₃(aq) (0.43 mL, 0.85 mmol). The mixture was degassed in a sonicator for 2 minutes. PdAmphos (120.37 mg, 0.17 mmol) was added, and the mixture heated to 90°C in a sealed vial for 1 hour. The reaction was quenched with water (5 mL) and extracted with ethyl acetate (2×10 mL) washed with brine, dried over MgSO₄ and concentrated in vacuo. The crude was dissolved in dichloromethane (5 mL). TFA (1 mL) was added, and the mixture was stirred for 60 minutes. The solvents were removed in vacuo. The crude was dissolved in THE (2 mL) and sat. NaHCO₃ (aq) (2 mL). Acryloyl chloride (15.1 μL, 0.18 mmoL) was added and reaction stirred until completion of reaction. This was quenched, diluted, and extracted with ethyl acetate (2×10 mL) washed with brine, dried over MgSO₄, concentrated in vacuo and purified by reversed phase HPLC to give 66 mg of the desired compound in 44% yield. m/z expected: 503.95, observed: 504.44; ¹H NMR (500 MHz DMSO) δ 11.30 (s, 1H), 8.93 (s, 1H), 8.80 (s, 1H), 8.42 (d, J=9.1 Hz, 1H), 8.34 (dd, J=2.2, 8.6 Hz, 1H), 8.30 (d, J=8.3 Hz, 1H), 8.25-8.20 (m, 2H), 7.95 (s, 1H), 6.89-6.83 (m, 1H), 6.17-6.08 (m, 1H), 5.74-5.67 (m, 1H), 4.68-4.61 (m, 2H), 4.32-4.30 (m, 1H), 3.29-3.05 (m, 2H), 2.32 (s, 1H), 2.19 (s, 1H), 1.87 (s, 1H), 1.62 (s, 1H).

### tert-butyl 6-(4-(6-(6-bromopicolinamido)pyridin-3-yl)-1H-1,2,3-triazol-1-yl)-2-azaspiro[3.3]heptane-2-carboxylate (36)

Prepared according to the procedure for 028 to give 250 mg of the desired product in 36% yield. m/z expected: 540.42, observed: 542.37; ¹H NMR (500 MHz DMSO) δ 10.24 (s, 1H), 8.86 (s, 1H), 8.80 (s, 1H), 8.33 (s, 1H), 8.21 (dd, J=7.5, 0.9 Hz, 1H), 8.05 (t, J=7.6 Hz, 1H), 7.98 (dd, J=8.0, 0.9 Hz, 1H), 5.11 (p, J=8.1 Hz, 1H), 4.00 (s, 2H), 3.90 (s, 2H), 2.86-2.82 (m, 2H), 2.73-2.70 (m, 2H), 1.38 (s, 9H)

### N-(5-(1-(2-acryloyl-2-azaspiro[3.3]heptan-6-yl)-1H-1,2,3-triazol-4-yl)pyridin-2-yl)-6-(4-chloro-1H-pyrazol-5-yl)picolinamide (Compound 027)

Prepared according to the procedure for 028 to give 17 mg in 21% yield. m/z expected: 515.96, observed: 516.46; ¹H NMR (500 MHz DMSO) δ 11.46 (s, 1H), 8.92 (s, 1H), 8.82 (s, 1H), 8.41 (d, J=8.6 Hz, 1H), 8.34-8.30 (m, 2H), 8.25-8.20 (m, 2H), 7.83 (s, 1H), 6.33-6.28 (m, 1H), 6.10 (d, J=16.9 Hz, 1H), 5.67 (dd, J=10.1, 8.2 Hz, 1H), 5.19-5.12 (m, 1H), 4.38 (s, 1H), 4.28 (s, 1H), 4.09 (s, 1H), 3.99 (s, 1H), 2.92-2.87 (m, 2H), 2.80-2.76 (m, 2H).

### (S)-N-(5-(1-(1-acryloylpiperidin-3-yl)-1H-1,2,3-triazol-4-yl)pyridin-2-yl)-6-(4-chloro-1H-pyrazol-5-yl)picolinamide (Compound 029)

Prepared according to the procedure for 028 to give 27 mg of the desired product in 33% yield. m/z expected: 503.95, observed: 504.44; ¹H NMR (500 MHz DMSO) δ 11.30 (s, 1H), 8.93 (s, 1H), 8.80 (s, 1H), 8.42 (d, J=8.6 Hz, 1H), 8.34 (dd, J=2.1, 8.6 Hz, 1H), 8.30 (d, J=7.3 Hz, 1H), 8.25-8.20 (m, 2H), 7.95 (s, 1H), 6.89-6.83 (m, 1H), 6.1-6.08 (m, 1H), 5.74-5.68 (m, 1H), 4.68-4.60 (m, 1H), 4.33-4.03 (m, 2H), 3.08 (s, 1H), 2.30 (s, 1H), 2.20 (s, 1H), 1.91 (s, 1H), 1.65 (s, 1H).

### N-(5-(1-(1-acryloyl-5,5-difluoropiperidin-3-yl)-1H-1,2,3-triazol-4-yl)pyridin-2-yl)-6-(1H-pyrazol-5-yl)picolinamide (Compound 036)

Prepared according to the procedure for 028 to give 8.3 mg in 30% yield. m/z expected: 505.49, observed: 506.46; ¹H NMR (500 MHz DMSO) δ 11.18 (s, 1H), 8.93 (s, 1H), 8.86 (s, 1H), 8.42 (d, J=8.6 Hz, 1H), 8.33 (dd, J=2.3, 8.6 Hz, 1H),), 8.15-8.13 (m, 3H), 7.75 (s, 1H), 7.07 (s, 1H), 7.00-6.92 (m, 1H), 6.26-6.20 (m, 1H), 5.82-5.80 (m, 1H), 4.95 (s, 1H), 4.81-4.6 (m, 1H), 4.63 (s, 1H), 4.53-4.51 (m, 1H), 2.93-2.86 (m, 3H).

### N-(5-(1-(1-acryloyl-5,5-difluoropiperidin-3-yl)-1H-1,2,3-triazol-4-yl)pyridin-2-yl)-6-(4-chloro-1H-pyrazol-5-yl)picolinamide (Compound 030)

Prepared according to the procedure for 028 to give 66 mg in 44% yield. m/z expected: 503.95, observed: 504.44; ¹H NMR (500 MHz DMSO) δ 11.36 (s, 1H), 8.92 (s, 1H), 8.85 (s, 1H), 8.43 (d, J=8.6 Hz, 1H), 8.33 (dd, J=2.3, 8.6 Hz, 1H), 8.31-8.30 (m, 1H), 8.25-8.20 (m, 2H), 8.06-7.97 (m, 1H), 7.01-6.90 (m, 1H), 6.26-6.20 (m, 1H), 5.82-5.80 (m, 1H), 4.93 (s, 1H), 4.79 (s, 1H), 4.63 (s, 1H), 4.53-4.51 (m, 1H), 3.56-3.38 (m, 1H), 2.93-2.86 (m, 2H).

### tert-butyl 7-azido-5-azaspiro[2.5]octane-5-carboxylate (41)

Prepared according to the procedure for 33 to give 300 mg that was carried forward without further purification. m/z expected: 236.16, expected (w/o tBu): 196.21, observed: 196.21; expected (w/o BOC): 152.32, observed: 153.19

### tert-butyl 7-(4-(6-(6-bromopicolinamido)pyridin-3-yl)-1H-1,2,3-triazol-1-yl)-5-azaspiro[2.5]octane-5-carboxylate (42)

Prepared according to the procedure for 34 to give 400 mg in 60% yield. m/z expected: 554.45, observed: 554.47; ¹H NMR (500 MHz DMSO) δ 10.27 (s, 1H), 8.86 (s, 1H), 8.79 (s, 1H), 8.36-8.32 (m, 2H), 8.22 (dd, J=0.9, 7.5 Hz, 1H), 8.06 (t, J=7.6 Hz, 1H), 7.99 (dd, J=0.9, 7.9 Hz, 1H), 4.69 (s, 1H), 4.32 (s,1H), 3.24 (s, 1H), 1.71-1.68 (m, 1H), 1.42 (s, 9H), 0.59-0.55 (m, 1H), 0.49-0.44 (m, 2H), 0.43-0.41 (m, 1H).

### N-(5-(1-(5-acryloyl-5-azaspiro[2.5]octan-7-yl)-1H-1,2,3-triazol-4-yl)pyridin-2-yl)-6-(1H-pyrazol-5-yl)picolinamide (Compound 031)

Prepared according to the procedure for 028 to give 67.3 mg in 50% yield. m/z expected: 495.55, observed: 495.97; ¹H NMR (500 MHz DMSO) δ 11.17 (s, 1H), 8.93 (s, 1H), 8.92 (s, 1H), 8.42 (d, J=8.6 Hz, 1H), 8.33 (d, J=8.6 Hz, 1H), 8.17-8.13 (m, 3H), 7.75 (s, 1H), 7.08 (s, 1H), 6.98-6.79 (m, 1H), 6.21-6.14 (m, 1H), 5.72 (dd, J=1.9, 10.1 Hz, 1H) 4.85-4.78 (m, 1H), 4.70-4.67 (m, 1H), 3.27-3.18 (m, 2H), 1.76-1.70 (m, 1H), 1.27-1.23 (m, 1H), 0.65-0.60 (m, 1H), 0.46 (m, 4H)

### N-(5-(1-(5-acryloyl-5-azaspiro[2.5]octan-7-yl)-1H-1,2,3-triazol-4-yl)pyridin-2-yl)-6-(4-chloro-1H-pyrazol-5-yl)picolinamide (Compound 032)

Prepared according to the procedure for 028 to give 12.3 mg in 30% yield. m/z expected 529.99, observed: 530.49.

### N-(4-(1-(3-acrylamidobicyclo[1.1.1]pentan-1-yl)-1H-1,2,3-triazol-4-yl)-2-methoxyphenyl)-6-(4-chloro-1H-pyrazol-5-yl)picolinamide (Compound 105)

Prepared according to the procedure for 028 to give 18.1 mg in 21.7% yield. LCMS (m/z): 531.0 [M+H]+. ¹H NMR (400 MHz, DMSO-d₆) δ 13.64 (s, 1H), 10.69-10.52 (m, 1H), 9.00 (s, 1H), 8.79-8.75 (m, 1H), 8.57-8.07 (m, 5H), 7.60 (d, J=1.6 Hz, 1H), 7.54 (dd, J=8.0, 1.6 Hz, 1H), 6.25-6.11 (m, 2H), 5.65 (dd, J=9.6, 2.8 Hz, 1H), 3.99 (s, 3H), 2.68 (s, 6H).

### N-(4-(1-(3-acrylamidobicyclo[1.1.1]pentan-1-yl)-1H-1,2,3-triazol-4-yl)-2-methoxyphenyl)-6-(1H-pyrazol-5-yl)picolinamide (Compound 106)

Prepared according to the procedure for 028 to give 48.7 mg in 54% yield. LCMS (m/z): 497.0 [M+H]+. ¹H NMR (400 MHz, DMSO-d₆) δ 13.27 (s, 1H), 10.89 (s, 1H), 9.00 (s, 1H), 8.79 (s, 1H), 8.51-7.96 (m, 5H), 7.63 (d, J=1.2 Hz, 1H), 7.57-7.54 (m, 1H), 7.01 (s, 1H), 6.26-6.11 (m, 2H), 5.66 (dd, J=9.6, 2.4 Hz, 1H), 4.10 (s, 3H), 2.68 (s, 6H).

### N-(4-(1-(3-acrylamidobicyclo[1.1.1]pentan-1-yl)-1H-1,2,3-triazol-4-yl)-2-methoxyphenyl)-6-(isoxazol-4-yl)picolinamide (Compound 107)

Prepared according to the procedure for 028 to give 6.4 mg in 8.4% yield. LCMS (m/z): 498.1 [M+H]+. ¹H NMR (400 MHz, DMSO) δ 11.02-10.83 (m, 1H), 9.23-6.88 (m, 9H), 6.27-6.07 (m, 2H), 5.76-5.59 (m, 1H), 4.02 (d, J=8.8 Hz, 3H), 2.67 (s, 6H).

### (S)-N-(4-(1-(1-acryloylpiperidin-3-yl)-1H-1,2,3-triazol-4-yl)-2-methoxyphenyl)-6-(1H-pyrazol-5-yl)picolinamide (Compound 112)

Prepared according to the procedure for 028 to give 16.4 mg in 8.3% yield. LCMS (m/z): 499.0 [M+H]+. ¹H NMR (400 MHz, DMSO-d₆) δ 13.24 (s, 1H), 10.85 (s, 1H), 8.75 (s, 1H), 8.42 (s, 1H), 8.22-8.06 (m, 3H), 7.90 (s, 1H), 7.63 (s, 1H), 7.53 (d, J=8.4 Hz, 1H), 7.00 (d, J=2.0 Hz, 1H), 6.87 (dd, J=16.4, 10.6 Hz, 1H), 6.18-6.10 (m, 1H), 5.71 (dd, J=16.8, 12.0 Hz, 1H), 4.78-4.29 (m, 2H), 4.15-4.03 (m, 4H), 3.28-3.00 (m, 2H), 2.36-2.12 (m, 2H), 1.95-1.80 (m, 1H), 1.70-1.52 (m, 1H).

### (S)-N-(4-(1-(1-acryloylpiperidin-3-yl)-1H-1,2,3-triazol-4-yl)-2-methoxyphenyl)-6-(isoxazol-4-yl)picolinamide (Compound 113)

Prepared according to the procedure for 028 to give 42.3 mg in 18.1% yield. LCMS (m/z): 500.0 [M+H]+. ¹H NMR (400 MHz, DMSO-d₆) δ 10.97-10.80 (m, 1H), 8.78-7.83 (m, 5H), 7.78-7.67 (m, 1H), 7.62-7.46 (m, 2H), 7.23-6.83 (m, 2H), 6.18-6.13 (m, 1H), 5.74-5.71 (m, 1H), 4.76-4.27 (m, 2H), 4.11-3.94 (m, 4H), 3.26-2.98 (m, 2H), 2.36-2.15 (m, 2H), 1.93-1.80 (m, 1H), 1.68-1.52 (m, 1H).

### (R)-N-(4-(1-(1-acryloylpiperidin-3-yl)-1H-1,2,3-triazol-4-yl)-2-methoxyphenyl)-6-(4-chloro-1H-pyrazol-5-yl)picolinamide (Compound 114)

Prepared according to the procedure for 028 to give 60 mg in 33% yield. LCMS (m/z): 533.0 [M+H]+. ¹H NMR (400 MHz, DMSO-d₆) δ 13.70 (s, 1H), 10.69 (s, 1H), 8.74 (s, 1H), 8.61-8.45 (m, 1H), 8.29-8.07 (m, 4H), 7.61-7.50 (m, 2H), 6.89-6.83 (m, 1H), 6.19-6.10 (m, 1H), 5.76-5.67 (m, 1H), 4.73-4.29 (m, 2H), 4.21-4.02 (m, 1H), 3.99 (s, 3H), 3.77-3.06 (m, 2H), 2.37-2.28 (m, 1H), 2.24-2.12 (m, 1H), 1.95-1.81 (m, 1H), 1.69-1.53 (m, 1H).

### (R)-N-(4-(1-(1-acryloylpiperidin-3-yl)-1H-1,2,3-triazol-4-yl)-2-methoxyphenyl)-6-(1H-pyrazol-5-yl)picolinamide (Compound 115)

Prepared according to the procedure for 028 to give 11.8 mg in 15% yield. LCMS (m/z): 499.2 [M+H]+. ¹H NMR (400 MHz, DMSO-d₆) δ 13.27 (s, 1H), 10.88 (s, 1H), 8.75 (s, 1H), 8.51 (s, 1H), 8.30-7.89 (m, 4H), 7.63 (s, 1H), 7.53 (d, J=8.4 Hz, 1H), 7.00 (s, 1H), 6.91-6.84 (m, 1H), 6.19-6.13 (m, 1H), 5.75-5.70 (m, 1H), 4.80-4.29 (m, 2H), 4.20-3.94 (m, 4H), 3.30-3.01 (m, 2H), 2.36-2.27 (m, 1H), 2.24-2.10 (m, 1H), 1.95-1.82 (m, 1H), 1.68-1.55 (m, 1H).

### (R)-N-(4-(1-(1-acryloylpiperidin-3-yl)-1H-1,2,3-triazol-4-yl)-2-methoxyphenyl)-6-(isoxazol-4-yl)picolinamide (Compound 116)

Prepared according to the procedure for 028 to give 16.5 mg in 21% yield. LCMS (m/z): 500.1 [M+H]+. ¹H NMR (400 MHz, DMSO-d₆) δ 10.97-10.80 (m, 1H), 8.83-7.85 (m, 5H), 7.75-7.70 (m, 1H), 7.61-6.10 (m, 5H), 5.78-5.65 (m, 1H), 4.79-4.09 (m, 3H), 4.03 (d, J=11.6 Hz, 3H), 3.23-2.98 (m, 2H), 2.36-2.10 (m, 2H), 1.95-1.78 (m, 1H), 1.67-1.50 (m, 1H).

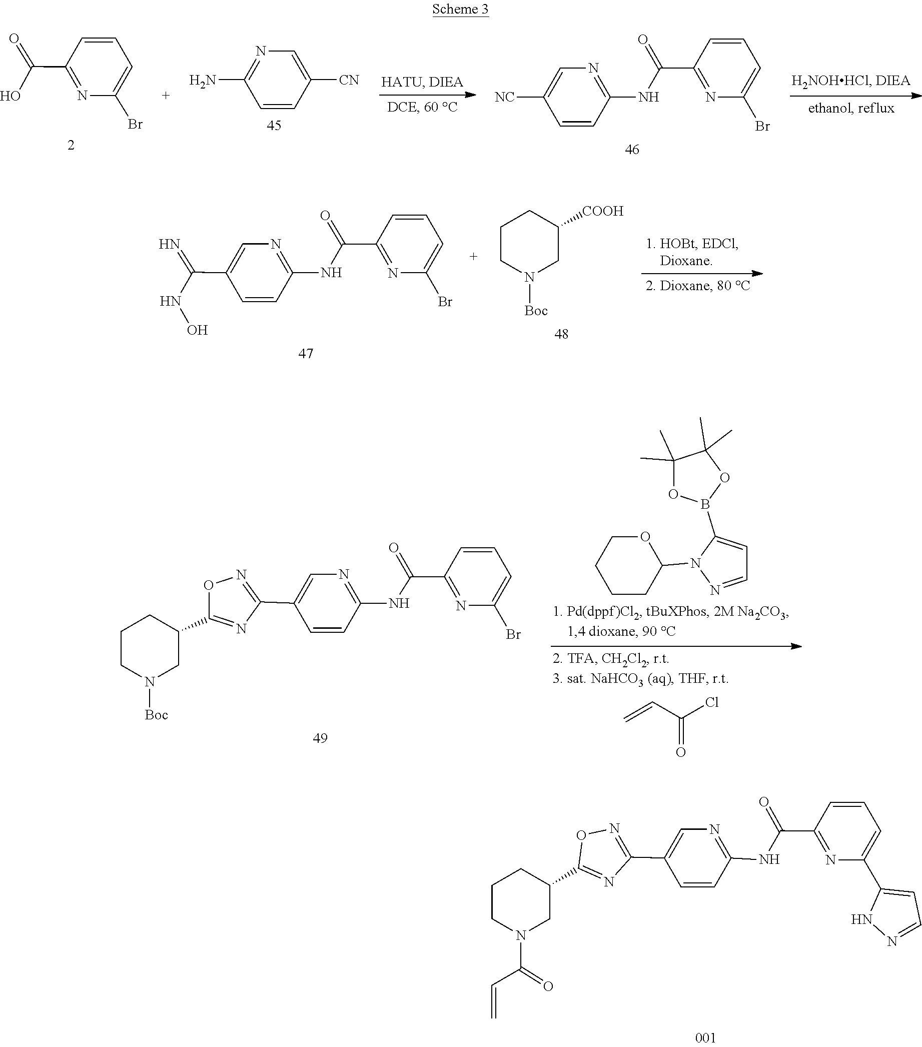

### 6-bromo-N-(5-cyanopyridin-2-yl)picolinamide (46)

Prepared according to the procedure for 6 to yield 2 g of a white solid that was carried forward without any further purification. m/z expected 303.12, observed: 303.14

### 6-bromo-N-(5-(N-hydroxycarbamimidoyl)pyridin-2-yl)picolinamide (47)

To 6-bromo-N-(5-cyanopyridin-2-yl)picolinamide (1.4 g, 4.62 mmol) in ethanol (50 mL) was added H₂NOH·HCl (962.8 mg, 13.85 mmol) and DIEA (2.41 mL, 13.85 mmol) and refluxed until consumption of starting material. The solvents were evaporated in vacuo, the crude diluted with ethyl acetate, washed with sat. NH₄Cl (aq), brine, dried over Na₂SO₄, concentrated to yield 1.2 g that was carried forward without purification. m/z expected 336.15, observed: 336.16

### tert-butyl (S)-3-(3-(6-(6-bromopicolinamido)pyridin-3-yl)-1,2,4-oxadiazol-5-yl)piperidine-1-carboxylate (49)

To a flask containing (S)-1-(tert-butoxycarbonyl)piperidine-3-carboxylic acid (500 mg, 2.18 mmol) in THE at 0°C was added EDCI (1.01 g, 6.54 mmol) and HOBt (589.27 mg, 4.36 mmol) and stirred for 15 minutes. 6-bromo-N-(5-(N-hydroxycarbamimidoyl)pyridin-2-yl)picolinamide (1.46 g, 4.36 mmol) was added and stirred at room temperature overnight. On completion of the reaction, the mixture was filtered to give the desired intermediate which was dissolved in 1,4 dioxane and heated at 80°C overnight. The reaction mixture was quenched with water diluted with ethyl acetate. The organic layers were washed with brine, dried over MgSO₄, concentrated. The crude was purified by flash chromatography using 25-100% ethyl acetate in hexanes to give 540 mg of the product in 47% yield. m/z expected: 529.4, observed: 529.68;

### (S)-N-(5-(5-(1-acryloylpiperidin-3-yl)-1,2,4-oxadiazol-3-yl)pyridin-2-yl)-6-(1H-pyrazol-5-yl)picolinamide (Compound 001)

Prepared according to the procedure for 028 to give 12.4 mg of the desired product in 32% yield. m/z expected: 470.49, observed: 471.09;

### (S)-N-(5-(5-(1-acryloylpiperidin-3-yl)-1,2,4-oxadiazol-3-yl)pyridin-2-yl)-6-(4-chloro-1H-pyrazol-5-yl)picolinamide (Compound 004)

Prepared according to the procedure for 001 to give 1.3 mg of the desired product in 6% yield. m/z expected: 504.94, observed: 505.37;

### (S)-N-(5-(5-(1-acryloylpiperidin-3-yl)-1,2,4-oxadiazol-3-yl)pyridin-2-yl)-6-(4-methyl-1H-pyrazol-5-yl)picolinamide (52 or Compound 006)

Prepared according to the procedure for 028 to give 30 mg of the desired product in 39% yield. m/z expected: 484.52, observed:

### tert-butyl (6S)-6-azido-2-azabicyclo[2.2.1]heptane-2-carboxylate (53)

Prepared according to the procedure for 33 to give 500 mg of the desired product in 22% yield. m/z expected: 238.29, expected: 239.23

### tert-butyl 6-(4-(6-(6-bromopicolinamido)pyridin-3-yl)-1H-1,2,3-triazol-1-yl)-2-azabicyclo[2.2.1]heptane-2-carboxylate (54)

Prepared according to the procedure for 34 to give 270 mg in 24% yield. m/z expected: 540.42, observed: 540.32;

### N-(5-(1-(2-acryloyl-2-azabicyclo[2.2.1]heptan-6-yl)-1H-1,2,3-triazol-4-yl)pyridin-2-yl)-6-(1H-pyrazol-5-yl)picolinamide (Compound 033)

Prepared according to the procedure for 038 to give 5 mg of the product in 10% yield. m/z expected: 481.52, observed: 481.82

### N-(5-(1-(2-acryloyl-2-azabicyclo[2.2.1]heptan-6-yl)-1H-1,2,3-triazol-4-yl)pyridin-2-yl)-6-(4-methyl-1H-pyrazol-5-yl)picolinamide (Compound 034)

Prepared according to the procedure for 038 to give 5 mg of the product in 9% yield. m/z expected: 481.52, observed: 481.82

### tert-butyl 5-(3-(6-(6-bromopicolinamido)pyridin-3-yl)-1,2,4-oxadiazol-5-yl)-3,3-difluoropiperidine-1-carboxylate (57)

Prepared according to the procedure for 49 to give 200 mg of the desired product in % yield. m/z expected: 565.38, observed: 565.31

### N-(5-(5-(1-acryloyl-5,5-difluoropiperidin-3-yl)-1,2,4-oxadiazol-3-yl)pyridin-2-yl)-6-(1H-pyrazol-5-yl)picolinamide (Compound 002)

Prepared according to the procedure for 038 to give 7.1 mg of the product in 29% yield. m/z expected: 506.47, observed: 507.34

### N-(5-(5-(1-acryloyl-5,5-difluoropiperidin-3-yl)-1,2,4-oxadiazol-3-yl)pyridin-2-yl)-6-(4-methyl-1H-pyrazol-5-yl)picolinamide (Compound 007)

Prepared according to the procedure for 038 to give 9.1 mg of the product in 37% yield. m/z expected: 520.5, observed: 521.37

### N-(5-(5-(1-acryloyl-5,5-difluoropiperidin-3-yl)-1,2,4-oxadiazol-3-yl)pyridin-2-yl)-6-(4-chloro-1H-pyrazol-5-yl)picolinamide (Compound 008)

Prepared according to the procedure for 038 to give 15.9 mg of the product in 39% yield. m/z expected: 540.92, observed: 541.32

### N-(5-(1-(2-acryloyl-2-azabicyclo[2.2.1]heptan-6-yl)-1H-1,2,3-triazol-4-yl)pyridin-2-yl)-6-(4-chloro-1H-pyrazol-5-yl)picolinamide (Compound 035)

Prepared according to the procedure for 001 to give 9 mg of the product in 14% yield. m/z expected: 515.96, observed: 516.2

### tert-butyl (S)-3-((6-(6-bromopicolinamido)-5-(trifluoromethyl)pyridin-3-yl)carbamoyl)piperidine-1-carboxylate (63)

Prepared according to the procedure for 49 to give 80 mg of the desired product in 25% yield. m/z expected: 572.38, observed:572.37;

### (S)-N-(5-(1-acryloylpiperidine-3-carboxamido)-3-(trifluoromethyl)pyridin-2-yl)-6-(1H-pyrazol-5-yl)picolinamide (Compound 050)

Prepared according to the procedure for 038 to give 14.4 mg of the product in 52% yield. m/z expected: 513.48, observed: 514.36

### tert-butyl (3S)-3-(3-(6-(6-bromopicolinamido)pyridin-3-yl)-1,2,4-oxadiazol-5-yl)-5-(trifluoromethyl)piperidine-1-carboxylate (65)

Prepared according to the procedure for 49 to give 75 mg of desired product in % yield. m/z expected: 597.39, observed: 597.28

### N-(5-(5-((3S)-1-acryloyl-5-(trifluoromethyl)piperidin-3-yl)-1,2,4-oxadiazol-3-yl)pyridin-2-yl)-6-(1H-pyrazol-5-yl)picolinamide (Compound 009)

Prepared according to the procedure for 038 to give 19.5 mg of desired product in 40% yield. m/z expected: 597.39, observed: 597.28

### tert-butyl (6S)-6-(3-(6-(6-bromopicolinamido)pyridin-3-yl)-1,2,4-oxadiazol-5-yl)-2-azabicyclo[2.2.1]heptane-2-carboxylate (67)

Prepared according to the procedure for 49 to give 930 mg of desired product in 42% yield. m/z expected: 541.41, observed: 541.32

### N-(5-(5-((6S)-2-acryloyl-2-azabicyclo[2.2.1]heptan-6-yl)-1,2,4-oxadiazol-3-yl)pyridin-2-yl)-6-(4-methyl-1H-pyrazol-5-yl)picolinamide (Compound 010)

Prepared according to the procedure for 038 to give 16.6 mg of desired product in 70% yield. m/z expected: 496.53, observed: 497.3

### N-(5-(5-((6S)-2-acryloyl-2-azabicyclo[2.2.1]heptan-6-yl)-1,2,4-oxadiazol-3-yl)pyridin-2-yl)-6-(1H-pyrazol-5-yl)picolinamide (Compound 011)

Prepared according to the procedure for 038 to give 9.6 mg of desired product in 41% yield. m/z expected: 482.5, observed: 482.9 tert-butyl (S)-3-(3-(6-(6-bromopicolinamido)-5-(trifluoromethyl)pyridin-3-yl)-1,2,4-oxadiazol-5-yl)piperidine-1-carboxylate (70)

Prepared according to the procedure for 49 to give 100 mg of desired product in 10% yield. m/z expected: 597.39, observed: 599.21

### (S)-N-(5-(5-(1-acryloylpiperidin-3-yl)-1,2,4-oxadiazol-3-yl)-3-(trifluoromethyl)pyridin-2-yl)-6-(1H-pyrazol-5-yl)picolinamide (Compound 013)

Prepared according to the procedure for 038 to give 10.9 mg of desired product in 39% yield. m/z expected: 538.49, observed: 539.64

### 6-bromo-N-(5-cyano-3-(difluoromethoxy)pyridin-2-yl)picolinamide (72)

To a stirring solution of 6-amino-5-(difluoromethoxy)nicotinonitrile (750 mg, 4.05 mmol) in dichloromethane was added DMAP(494.93 mg, 4.05 mmol), 6-bromopicolinoyl chloride (2 g, 9.1 mmol). DIEA (3.52 mL, 20.25 mmol) was added dropwise and reaction stirred until completion. The reaction was quenched with water, diluted with DCM, organic layers were washed with sat. NH₄Cl (aq), brine, dried over Na₂SO₄, concentrated and the crude was purified by flash chromatography (0-20% ethyl acetate in DCM) to give 900 mg of the desired product in 50% yield. m/z expected: 369.13, observed: 369.10

### 6-bromo-N-(3-(difluoromethoxy)-5-(N-hydroxycarbamimidoyl)pyridin-2-yl)picolinamide (73)

Prepared according to the procedure for 47 to give 400 mg that was carried forward without further purification. m/z expected: 402.16, observed: 402.11

### tert-butyl (S)-3-(3-(6-(6-bromopicolinamido)-5-(difluoromethoxy)pyridin-3-yl)-1,2,4-oxadiazol-5-yl)piperidine-1-carboxylate (74)

Prepared according to the procedure for 49 to give 100 mg of desired product in 10% yield. m/z expected: 595.4, observed: 596.79

### (S)-N-(5-(5-(1-acryloylpiperidin-3-yl)-1,2,4-oxadiazol-3-yl)-3-(difluoromethoxy)pyridin-2-yl)-6-(1H-pyrazol-5-yl)picolinamide (Compound 014)

Prepared according to the procedure for 038 to give 10 mg of the product in 22.4% yield. m/z expected: 536.5, observed: 537.33

### 6-bromo-N-(5-cyano-3-(trifluoromethoxy)pyridin-2-yl)picolinamide (76)

Prepared according to the procedure for 72 to give 820 mg of the desired product in 78% yield. m/z expected: 387.12, observed: 387.12

### 6-bromo-N-(5-(N-hydroxycarbamimidoyl)-3-(trifluoromethoxy)pyridin-2-yl)picolinamide (77)

Prepared according to the procedure for 47 to give 400 mg that was carried forward without further purification. m/z expected: 420.15, observed: 420.09

### tert-butyl (S)-3-(3-(6-(6-bromopicolinamido)-5-(trifluoromethoxy)pyridin-3-yl)-1,2,4-oxadiazol-5-yl)piperidine-1-carboxylate (78)

Prepared according to the procedure for 49 to give mg of desired product in 10% yield. m/z expected: 613.39, observed: 613.28

### (S)-N-(5-(5-(1-acryloylpiperidin-3-yl)-1,2,4-oxadiazol-3-yl)-3-(trifluoromethoxy)pyridin-2-yl)-6-(1H-pyrazol-5-yl)picolinamide (Compound 015)

Prepared according to the procedure for 038 to give 9.4 mg of the product in 21% yield. m/z expected: 554.16, observed: 555.31

### (S)-N-(5-(5-(1-acryloylpiperidin-3-yl)-1,2,4-oxadiazol-3-yl)pyridin-2-yl)-6-(1-(2,2,2-trifluoroethyl)-1H-pyrazol-4-yl)picolinamide (Compound 012)

Prepared according to the procedure for 038 to give 19.5 mg of the product in 53% yield. m/z expected: 552.52, observed: 553.34

### (S)-N-(5-(5-(1-acryloylpiperidin-3-yl)-1,2,4-oxadiazol-3-yl)pyridin-2-yl)-6-(1-methyl-1H-pyrazol-4-yl)picolinamide (Compound 051)

Prepared according to the procedure for 038 to give 19.3 mg of the product in 49% yield. m/z expected: 484.52, observed: 485.33

### 5-(4,4,5,5-tetramethyl-1,3,2-dioxaborolan-2-yl)-1-(2,2,2-trifluoroethyl)-1H-pyrazole (82)

To a solution of 5-(4,4,5,5-tetramethyl-1,3,2-dioxaborolan-2-yl)-1H-pyrazole (1 g, 5.15 mmol) in DMF was added 2,2,2-trifluoroethyl trifluoromethanesulfonate (5.2 mL, 30.9 mmol), Cs₂CO₃ (3.35 g, 10.3 mmol) and stirred at 100°C. The reaction was quenched with water, diluted with ethyl acetate, washed with brine, dried over Na₂SO₄, concentrated and the crude was carried forward without further purification. m/z expected: 276.07, observed: 276.87

### (S)-N-(5-(5-(1-acryloylpiperidin-3-yl)-1,2,4-oxadiazol-3-yl)pyridin-2-yl)-6-(1-(2,2,2-trifluoroethyl)-1H-pyrazol-5-yl)picolinamide (Compound 016)

Prepared according to the procedure for 038 to give 49.4 mg of the product in 73% yield. m/z expected: 552.52, observed: 553.4

### tert-butyl (S)-3-(3-(6-(6-bromopicolinamido)pyridin-3-yl)-1,2,4-oxadiazol-5-yl)piperidine-1-carboxylate (84)

Prepared according to the procedure for 49 to give 42 mg of desired product in 84% yield. m/z expected: 533.61, observed: 534.77

### (S)-N-(5-(5-(1-acryloylpiperidin-3-yl)-1,2,4-oxadiazol-3-yl)pyridin-2-yl)-6-(thiazol-5-yl)picolinamide (Compound 052)

Prepared according to the procedure for 038 to give 19.6 mg of the product in 54% yield. m/z expected: 487.54, observed: 488.6

### 4-(4,4,5,5-tetramethyl-1,3,2-dioxaborolan-2-yl)-1-((1-(trifluoromethyl)cyclopropyl)methyl)-1H-pyrazole (86)

To a vial containing the solvent Toluene:THF (4:1) at 0°C was added PPh₃ (187.27 mg, 0.71 mmol) and DIAD (0.15 mL, 0.71 mmol) dropwise and stirred until a pale yellow suspension was formed. To this was added a cooled solution of (1-(trifluoromethyl)cyclopropyl)methanol (79.38 mg, 0.57 mmol) and 4-(4,4,5,5-tetramethyl-1,3,2-dioxaborolan-2-yl)-1H-pyrazole (100 mg, 0.51 mmol) in Toluene:THF (4:1) and the reaction mixture stirred at room temperature until completion of the reaction. The reaction was quenched with water, diluted with ethyl acetate, washed with brine, dried over Na₂SO₄, concentrated and the crude was purified by flash chromatography (20-70% ethyl acetate in hexanes) to give 462 mg of desired product in 71% yield. m/z expected: 316.13, observed: 317.3

### tert-butyl (S)-3-(3-(6-(6-(1-((1-(trifluoromethyl)cyclopropyl)methyl)-1H-pyrazol-4-yl)picolinamido)pyridin-3-yl)-1,2,4-oxadiazol-5-yl)piperidine-1-carboxylate (87)

Prepared according to the procedure for 13 to give 50 mg of the desired product in 83% yield. m/z expected: 638.65, observed: 639.45

### (S)-N-(5-(5-(1-acryloylpiperidin-3-yl)-1,2,4-oxadiazol-3-yl)pyridin-2-yl)-6-(1-((1-(trifluoromethyl)cyclopropyl)methyl)-1H-pyrazol-4-yl)picolinamide (Compound 053)

Prepared according to the procedure for 038 to give 21.3 mg of the desired product in 46% yield. m/z expected: 592.5, observed: 593.5

### (S)-N-(5-(5-(1-acryloylpiperidin-3-yl)-1,2,4-oxadiazol-3-yl)pyridin-2-yl)-6-(1-((1-(trifluoromethyl)cyclopropyl)methyl)-1H-pyrazol-5-yl)picolinamide (Compound 017)

Prepared according to the procedure for 038 to give 6.6 mg of the desired product in 20% yield. m/z expected: 592.5, observed: 593.4

### tert-butyl (S)-3-(3-(6-(6-bromopicolinamido)pyridin-3-yl)-1,2,4-oxadiazol-5-yl)piperidine-1-carboxylate (90)

Prepared according to the procedure for 49 to give 27.8 mg of the desired product in 53% yield. m/z expected: 556.63, observed: 557.75

### (S)-N-(5-(5-(1-acryloylpiperidin-3-yl)-1,2,4-oxadiazol-3-yl)pyridin-2-yl)-6-(1-cyclopropyl-1H-pyrazol-4-yl)picolinamide (Compound 054)

Prepared according to the procedure for 038 to give 13.3 mg of the desired product in 54% yield. m/z expected: 510.56, observed: 511.37

### tert-butyl (S)-3-(3-(6-(6-bromopicolinamido)pyridin-3-yl)-1,2,4-oxadiazol-5-yl)piperidine-1-carboxylate (92)

Prepared according to the procedure for 49 to give 35 mg of the desired product in 68% yield. m/z expected: 533.61, observed: 534.1

### (S)-N-(5-(5-(1-acryloylpiperidin-3-yl)-1,2,4-oxadiazol-3-yl)pyridin-2-yl)-6-(thiazol-4-yl)picolinamide (93 or Compound 055)

Prepared according to the procedure for 13 to give 5.2 mg of the desired product in 16% yield. m/z expected: 487.54, observed: 488.30.

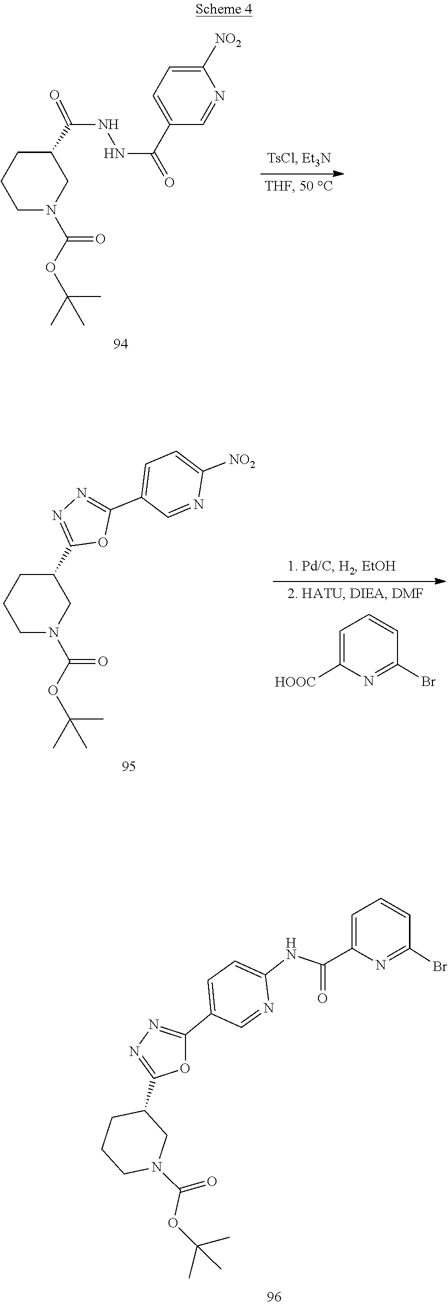 [SMILES: CC(C)(C)OC(=O)N1CCC[C@H](C(=O)NNC(=O)c2ccc([N+](=O)[O-])nc2)C1 ; CC(C)(C)OC(=O)N1CCC[C@H](c2nnc(-c3ccc([N+](=O)[O-])nc3)o2)C1 ; O=C(O)c1cccc(Br)n1 ; CC(C)(C)OC(=O)N1CCC[C@H](c2nnc(-c3ccc(NC(=O)c4cccc(Br)n4)nc3)o2)C1]

### tert-butyl (S)-3-(2-(6-nitronicotinoyl)hydrazine-1-carbonyl)piperidine-1-carboxylate (94)

Prepared according to the procedure for 15 to give 750 mg of the desired product in 47% yield. m/z expected: 393.4, observed: 394.3

### tert-butyl (S)-3-(5-(6-nitropyridin-3-yl)-1,3,4-oxadiazol-2-yl)piperidine-1-carboxylate (95)

To a solution of tert-butyl (S)-3-(2-(6-nitronicotinoyl)hydrazine-1-carbonyl)piperidine-1-carboxylate (750 mg, 1.90 mmol) in THE was added tosyl chloride (1.09 g, 5.7 mmol), T.E.A (1.59 mL, 11.4 mmol) and stirred at 50°C until completion of reaction. The solvents were evaporated in vacuo, the crude diluted with ethyl acetate, quenched with water, washed with brine, dried over Na₂SO₄, condensed and purified by flash chromatography (10-70% ethyl acetate in hexanes) to yield 510 mg of the desired product in 72% yield. m/z expected: 375.39, observed: 376.36

### tert-butyl (S)-3-(5-(6-(6-bromopicolinamido)pyridin-3-yl)-1,3,4-oxadiazol-2-yl)piperidine-1-carboxylate (96)

To a solution of tert-butyl (S)-3-(5-(6-nitropyridin-3-yl)-1,3,4-oxadiazol-2-yl)piperidine-1-carboxylate (500 mg, 1.33 mmol) in ethanol was added Pd on C (141.54 mg, 0.13 mmol) and stirred at 50°C under hydrogen. On completion of the reaction, the reaction mixture was filtered through celite and condensed. The crude was subjected to an amide coupling according to the procedure for the compound to give 240 mg of the product in 34% yield. m/z expected: 529.4, observed:

### (S)-N-(5-(5-(1-acryloylpiperidin-3-yl)-1,3,4-oxadiazol-2-yl)pyridin-2-yl)-6-(1H-pyrazol-5-yl)picolinamide (Compound 005)

Prepared according to the procedure for 038 to give 14.5 mg of the desired product in 31% yield. m/z expected: 470.49, observed: 470.85.

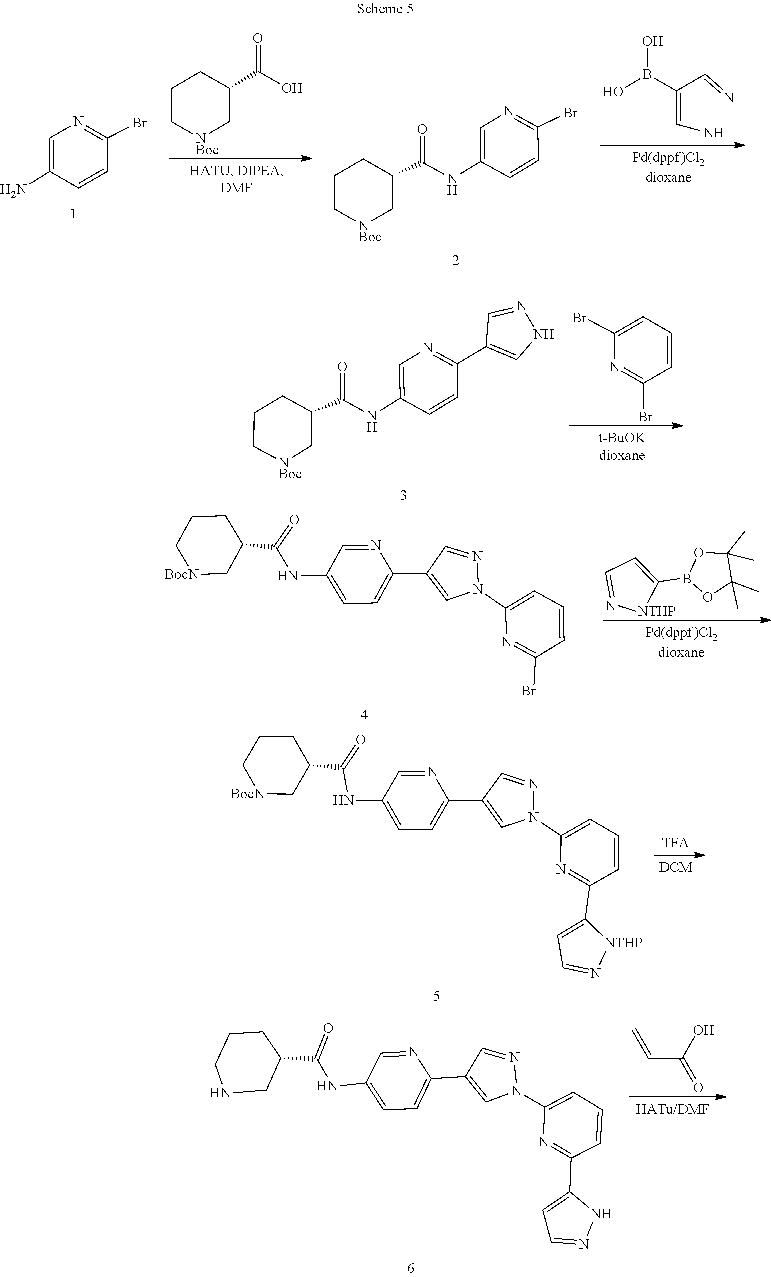

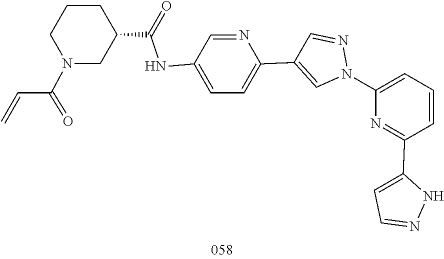 [SMILES: C=CC(=O)N1CCC[C@H](C(=O)Nc2ccc(-c3cnn(-c4cccc(-c5ccn[nH]5)n4)c3)nc2)C1]

### Synthesis of tert-butyl (S)-3-((6-bromopyridin-3-yl)carbamoyl)piperidine-1-carboxylate (2)

To a solution of 6-bromopyridin-3-amine (500 mg, 2.91 mmol) in DMF (10 mL) was added (S)-1-(tert-butoxycarbonyl)piperidine-3-carboxylic acid (798.8 mg, 3.49 mmol), HATU (1.66 g, 4.36 mmol), DIPEA (562.5 mg, 4.36 mmol). The resulting mixture was stirred at room temperature overnight, and then diluted with water (20 mL), the aqueous phase was extracted with ethyl acetate (30 mL×3). The combined organic phase was concentrated and the residue was purified by flash chromatography (eluting with petrol ether: ethyl acetate=0-52%) to give compound 2 (1 g, white solid, yield 89.8%). LCMS (m/z): 384.0 [M+H]⁺.

### Synthesis of tert-butyl (S)-3-((6-(1H-pyrazol-4-yl)pyridin-3-yl)carbamoyl)piperidine-1-carboxylate (3)

To a solution of tert-butyl (S)-3-((6-bromopyridin-3-yl)carbamoyl)piperidine-1-carboxylate (1 g, 2.61 mmol) in 1,4-dioxane/water (20 mL/5 mL) was added (1H-pyrazol-4-yl)boronic acid (467.9 mg, 4.18 mmol), Pd(dppf)C₁₂ (191.1 mg, 0.26 mmol) and K₂CO₃ (720.6 mg, 5.22 mmol) The resulting mixture was stirred at 95°C overnight under nitrogen atmosphere, and then concentrated. The residue was purified by flash chromatography (eluting with dichloromethane: methanol=0-20%) to give compound 3 (700 mg, yellow oil, yield72.3%). LCMS (m/z): 372.2 [M+H]⁺.

### Synthesis of tert-butyl (S)-3-((6-(1-(6-bromopyridin-2-yl)-1H-pyrazol-4-yl)pyridin-3-yl)carbamoyl)piperidine-1-carboxylate (4)

To a solution of tert-butyl (S)-3-((6-(1H-pyrazol-4-yl)pyridin-3-yl)carbamoyl)piperidine-1-carboxylate (700 mg, 1.89 mmol) in 1,4-dioxane (15 mL) was added 2,6-dibromopyridine (598.6 mg, 2.55 mmol) and t-BuOK (295.8 mg, 2.64 mmol) The resulting mixture was stirred at 100°C overnight and concentrated. The residue was purified by flash chromatography (eluting with petrol ether: ethyl acetate=0-100%) to give compound 4 (350 mg, yellow oil, yield 63%). LCMS (m/z): 527.2 [M+H]⁺.

### Synthesis of tert-butyl (3S)-3-((6-(1-(6-(1-(tetrahydro-2H-pyran-2-yl)-1H-pyrazol-5-yl)pyridin-2-yl)-1H-pyrazol-4-yl)pyridin-3-yl)carbamoyl)piperidine-1-carboxylate (5)

To a solution of tert-butyl (S)-3-((6-(1-(6-bromopyridin-2-yl)-1H-pyrazol-4-yl)pyridin-3-yl)carbamoyl)piperidine-1-carboxylate (350 mg, 0.67 mmol) in 1,4-dioxane/water (20 mL/5 mL) was added 1-(tetrahydro-2H-pyran-2-yl)-5-(4,4,5,5-tetramethyl-1,3,2-dioxaborolan-2-yl)-1H-pyrazole (240.5 mg, 0.87 mmol), Pd(dppf)Cl₂ (24.4 mg, 0.033 mmol) and K₂CO₃ (165.3 mg, 1.12 mmol). The resulting mixture was stirred at 95°C overnight under nitrogen atmosphere, and then concentrated. The residue was purified by flash chromatography (eluting with dichloromethane: methanol=0-10%) to give compound 5 (280 mg, yellow oil, yield 70.5%). LCMS (m/z): 599.3 [M+H]⁺.

### Synthesis of (S)-N-(6-(1-(6-(1H-pyrazol-5-yl)pyridin-2-yl)-1H-pyrazol-4-yl)pyridin-3-yl)piperidine-3-carboxamide (6)

To a solution of tert-butyl (3S)-3-((6-(1-(6-(1-(tetrahydro-2H-pyran-2-yl)-1H-pyrazol-5-yl)pyridin-2-yl)-1H-pyrazol-4-yl)pyridin-3-yl)carbamoyl)piperidine-1-carboxylate (280 mg, 0.47 mmol) in dichloromethane (7 mL) was added TFA (4 mL). The resulting mixture was stirred at room temperature for 2 hs and concentrated to give compound 6 (180 mg, brown oil, crude), which was used directly for the next stepLCMS (m/z): 415.2 [M+H]⁺.

### Synthesis of (S)-N-(6-(1-(6-(1H-pyrazol-5-yl)pyridin-2-yl)-1H-pyrazol-4-yl)pyridin-3-yl)-1-acryloylpiperidine-3-carboxamide (Compound 058)

To a solution of (S)-N-(6-(1-(6-(1H-pyrazol-5-yl)pyridin-2-yl)-1H-pyrazol-4-yl)pyridin-3-yl)piperidine-3-carboxamide (180 mg, crude) in DMF (6 mL) was added acrylic acid (62.8 mg, 0.87 mmol), HATU (414 mg, 1.09 mmol), DIPEA (140.6 mg, 1.09 mmol). The resulting mixture was stirred at room temperature overnight, and then diluted with water (15 ml), the aqueous phase was extracted with ethyl acetate (15 mL×3). The combined organic phase was concentrated and the residue was purified by Prep-HPLC to give compound DFCI-001-7 (3.8 mg, white solid, yield 2% two step). LCMS (m/z): 469.1 [M+H]⁺. ¹H NMR (400 MHz, DMSO-d₆) δ 13.62-13.42 (m, 1H), 10.34 (s, 1H), 9.74-9.28 (m, 1H), 8.78 (s, 1H), 8.37 (s, 1H), 8.14 (d, J=8.0 Hz, 1H), 8.09-8.00 (m, 1H), 7.97-7.71 (m, 4H), 7.21-7.02 (m, 1H), 6.97-6.78 (m, 1H), 6.12 (dd, J=16.8, 2.0 Hz, 1H), 5.73-5.65 (m, 1H), 4.68-4.45 (m, 1H), 4.33-4.13 (m, 1H), 4.08-3.99 (m, 1H), 3.14-2.73 (m, 2H), 2.06-1.98 (m, 1H), 1.83-1.69 (m, 2H), 1.42-1.35 (m, 1H).

### (S)-N-(4-(1-(6-(1H-pyrazol-5-yl)pyridin-2-yl)-1H-pyrazol-4-yl)phenyl)-1-acryloylpiperidine-3-carboxamide (Compound 072)

Prepared according to the procedure for 058 to give 29 mg of the desired product in 9.3% yield. LCMS (m/z): 468.2 [M+H]⁺. ¹H NMR (400 MHz, DMSO-d₆) δ 13.70-13.14 (m, 1H), 10.09 (s, 1H), 9.60-9.20 (m, 1H), 8.29 (s, 1H), 8.09-7.98 (m, 1H), 7.90 (d, J=6.4 Hz, 1H), 7.85-7.65 (m, 6H), 7.22-7.06 (m, 1H), 6.96-6.78 (m, 1H), 6.12 (d, J=16.8 Hz, 1H), 5.73-5.62 (m, 1H), 4.57-4.27 (m, 1H), 4.20-4.00 (m, 1H), 3.29-3.05 (m, 1H), 2.88-2.56 (m, 2H), 2.03-1.96 (m, 1H), 1.83-1.67 (m, 2H), 1.43-1.32 (m, 1H).

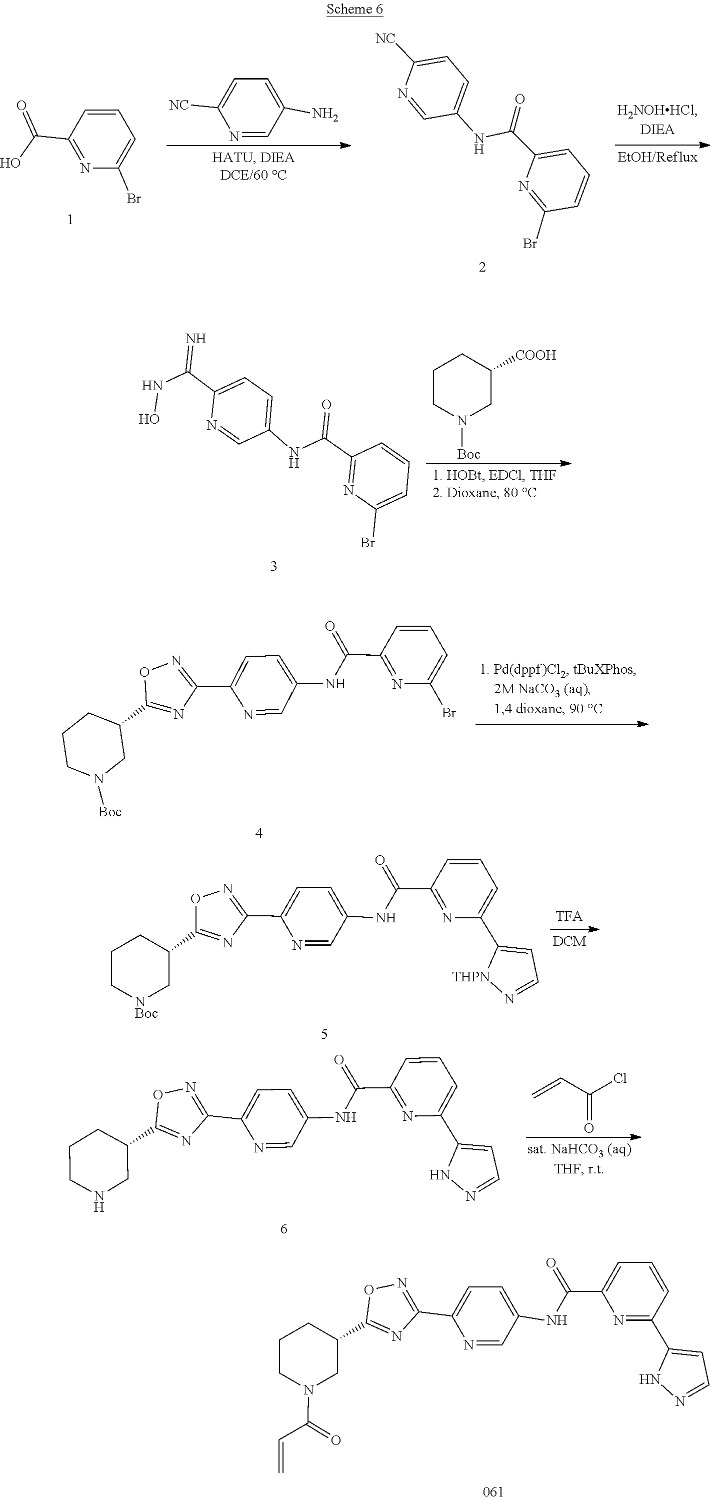

### Synthesis of 6-bromo-N-(6-cyanopyridin-3-yl)picolinamide (2)

To a solution of 6-bromopicolinic acid (1 g, 4.98 mmol) in 1,2-dichloroethane (15 mL) was added 5-aminopicolinonitrile (514 mg, 4.32 mmol), HATU (3.4 g, 8.96 mmol), DIPEA (1.16 g, 8.96 mmol). The resulting mixture was stirred at 60°C overnight and added water (15 mL), the aqueous phase was extracted with dichloromethane (15 mL×3). The combined organic phase was concentrated, the residue was purified by flash chromatography (eluting with dichloromethane: methanol=0-5%) to give compound 2 (629 mg, yellow oil, yield 41.9%). LCMS (m/z): 303.0 [M+H]⁺.

### Synthesis of 6-bromo-N-(6-(N-hydroxycarbamimidoyl)pyridin-3-yl)picolinamide (3)

To a solution of 6-bromo-N-(6-cyanopyridin-3-yl)picolinamide(629 mg, 2.08 mmol) in ethanol (20 ml) was added hydroxylamine hydrochloride (431 mg, 6.25 mmol) and DIPEA (806 mg, 6.25 mmol). The resulting mixture was stirred at 80°C overnight, and then diluted with aq. sat. NH₄Cl (20 mL), and the aqueous phase was extracted with ethyl acetate (15 mL×3). The combined organic phase was washed with brine (30 mL), dried by Na₂SO₄ and concentrated to give compound 3 (580 mg, white solid, crude), which was used directly for the next step. LCMS (m/z): 336.0 [M+H]⁺.

### Synthesis of tert-butyl (S)-3-(3-(5-(6-bromopicolinamido)pyridin-2-yl)-1,2,4-oxadiazol-5-yl)piperidine-1-carboxylate (4)

To a solution (S)-1-(tert-butoxycarbonyl)piperidine-3-carboxylic acid (467 mg, 2.04 mmol) in THE (15 mL) was added EDCI (1.17 g, 6.13 mmol) and HOBt (551 mg, 4.08 mmol) and the mixture was stirred at room temperature for 15 min. 6-bromo-N-(6-(N-hydroxycarbamimidoyl)pyridin-3-yl)picolinamide (580 mg, crude) in THE (5 mL) was added and the mixture was stirred at room temperature under nitrogen atmosphere overnight. The clear liquid was filtered and concentrated, the resiude was dissovled with 1,4-dioxane (20 mL), and the mixture was continue stirred at 80°C overnight and concentrated. The residue was purified by flash chromatography (eluting with petroleum ether: ethyl acetate=0-50%) to give compound 5 (580 mg, yellow oil, yield 52.8% two step). LCMS (m/z): 529.2 [M+H]⁺.

### Synthesis of tert-butyl (3S)-3-(3-(5-(6-(1-(tetrahydro-2H-pyran-2-yl)-1H-pyrazol-5-yl)picolinamido)pyridin-2-yl)-1,2,4-oxadiazol-5-yl)piperidine-1-carboxylate (5)

To a solution of tert-butyl (S)-3-(3-(5-(6-bromopicolinamido)pyridin-2-yl)-1,2,4-oxadiazol-5-yl)piperidine-1-carboxylate (530 mg, 1.00 mmol) in 1,4-dioxane/2N Na₂CO₃ (20 mL/5 mL) was added 1-(tetrahydro-2H-pyran-2-yl)-5-(4,4,5,5-tetramethyl-1,3,2-dioxaborolan-2-yl)-1H-pyrazole (363 mg, 1.31 mmol), Pd(dppf)Cl₂ (73.5 mg, 0.10 mmol) and t-BuXPhos(85.3 mg, 0.20 mmol). The resulting mixture was stirred at 95°C overnight under nitrogen atmosphere and concentrated. The residue was purified by flash chromatography (eluting with petroleum ether: ethyl acetate=0-52%) to give compound 5 (280 mg, yellow oil, yield 46.5%). LCMS (m/z): 601.3 [M+H]⁺.

### Synthesis of (S)-N-(6-(5-(piperidin-3-yl)-1,2,4-oxadiazol-3-yl)pyridin-3-yl)-6-(1H-pyrazol-5-yl)picolinamide (6)

To a solution tert-butyl (3S)-3-(3-(5-(6-(1-(tetrahydro-2H-pyran-2-yl)-1H-pyrazol-5-yl)picolinamido)pyridin-2-yl)-1,2,4-oxadiazol-5-yl)piperidine-1-carboxylate (280 mg, 0.47 mmol) in dichloromethane (7 mL) was added TFA (4 mL). The resulting mixture was stirred at room temperature for 2 hs and concentrated to give compound 6 (200 mg, yellow oil, crude), which was used directly for the next step. LCMS (m/z): 417.2 [M+H]⁺.

### Synthesis of (S)-N-(6-(5-(1-acryloylpiperidin-3-yl)-1,2,4-oxadiazol-3-yl)pyridin-3-yl)-6-(1H-pyrazol-5-yl)picolinamide (Compound 061)

To a solution of (S)-N-(6-(5-(piperidin-3-yl)-1,2,4-oxadiazol-3-yl)pyridin-3-yl)-6-(1H-pyrazol-5-yl)picolinamide (200 mg, crude) and aq. sat. NaHCO₃ (3 mL) in THE (3 mL) at 0°C was added dropwisely a solution of acryloyl chloride (60 mg, 0.66 mmol) in THE (1 mL). The resulting mixture was stirred at 0°C for 1 h, and diluted with water (10 mL), the aqueous phase was extracted by ethyl acetate (15 mL×3). The combined organic phase was concentrated, the residue was purified by Prep-HPLC to give compound DFCI-001-14 (86.4 mg, white solid, two steps yield 39.4%). LCMS (m/z): 471.1 [M+H]⁺. ¹H NMR (400 MHz, DMSO-d₆) δ 14.31-12.87 (m, 1H), 11.32-10.69 (m, 1H), 9.23 (s, 1H), 8.59 (d, J=8.4 Hz, 1H), 8.27-7.04 (m, 6H), 6.96-6.80 (m, 1H), 6.11 (t, J=16.8 Hz, 1H), 5.70 (d, J=9.6 Hz, 1H), 4.69-3.72 (m, 3H), 3.32-3.18 (m, 2H), 2.29-2.17 (m, 1H), 2.06-1.67 (m, 2H), 1.64-1.50 (m, 1H).

### (S)-N-(4-(5-(piperidin-3-yl)-1,2,4-oxadiazol-3-yl)phenyl)-6-(1H-pyrazol-5-yl)picolinamide (Compound 075)

Prepared according to the procedure for 061 to give 13.3 mg of the desired product in 24.3% yield. LCMS (m/z): 470.2 [M+H]⁺. ¹H NMR (400 MHz, DMSO-d₆) 514.31-12.87 (m, 1H), 11.09-10.54 (m, 1H), 8.26-7.46 (m, 9H), 6.98-6.81 (m, 1H), 6.11 (t, J=14.6 Hz, 1H), 5.70 (d, J=10.4 Hz, 1H), 4.64-3.80 (m, 3H), 3.31-3.23 (m, 2H), 2.30-2.15 (m, 1H), 2.04-1.69 (m, 2H), 1.62-1.50 (m, 1H).

### (S)-N-(4-(5-(1-acryloylpiperidin-3-yl)-1,2,4-oxadiazol-3-yl)-2-fluorophenyl)-6-(1H-pyrazol-5-yl)picolinamide (Compound 076)

Prepared according to the procedure for 061 to give 19.6 mg of the desired product in 4.4% yield. LCMS (m/z): 488.1 [M+H]⁺. ¹H NMR (400 MHz, DMSO-d₆) δ 13.98-13.24 (m, 1H), 10.94-10.73 (m, 1H), 8.50-7.61 (m, 7H), 7.18-7.05 (m, 1H), 6.98-6.79 (m, 1H), 6.11 (t, 1H), 5.70 (d, J=10.0 Hz, 1H), 4.73-3.65 (m, 3H), 3.31-3.08 (m, 2H), 2.29-2.16 (m, 1H), 2.05-1.66 (m, 2H), 1.61-1.50 (m, 1H).

### (S)-N-(4-(5-(1-acryloylpiperidin-3-yl)-1,2,4-oxadiazol-3-yl)-2-chlorophenyl)-6-(1H-pyrazol-5-yl)picolinamide (Compound 079)

Prepared according to the procedure for 061 to give 152.4 mg of the desired product in 21.3% yield. LCMS (m/z): 504.0 [M+H]⁺. ¹H NMR (400 MHz, DMSO) δ 13.58 (m, 1H), 11.03-10.99 (m, 1H), 8.76-7.62 (m, 7H), 7.04 (s, 1H), 6.96-6.81 (m, 1H), 6.12 (dd, J=16.4, 8.0 Hz, 1H), 5.71 (d, J=10.4 Hz, 1H), 4.66-3.73 (m, 3H), 3.46-3.38 (m, 1H), 3.29-3.21 (m, 1H), 2.29-2.15 (m, 1H), 2.05-1.64 (m, 2H), 1.59-1.49 (m, 1H).

### (S)-N-(4-(5-(1-acryloylpiperidin-3-yl)-1,2,4-oxadiazol-3-yl)-2-(trifluoromethyl)phenyl)-6-(1H-pyrazol-5-yl)picolinamide (Compound 080)

Prepared according to the procedure for 061 to give 16 mg of the desired product in 14% yield. LCMS (m/z): 538.2 [M+H]⁺. ¹H NMR (400 MHz, MeOD-d₄) δ 8.80 (s, 1H), 8.42-8.32 (m, 2H), 8.25 (s, 1H), 8.20-8.15 (m, 1H), 8.13-8.05 (m, 1H), 7.81 (s, 1H), 7.08 (s, 1H), 6.98-6.76 (m, 1H), 6.21 (dd, J=16.8, 2.0 Hz, 1H), 5.77 (dd, J=10.8, 2.0 Hz, 1H), 4.67-4.10 (m, 1H), 4.07-3.96 (m, 1H), 3.90-3.56 (m, 1H), 3.54-3.36 (m, 2H), 2.40-2.23 (m, 1H), 2.21-1.83 (m, 2H), 1.74-1.62 (m, 1H).

### (S)-N-(4-(5-(1-acryloylpiperidin-3-yl)-1,2,4-oxadiazol-3-yl)-2-(trifluoromethoxy)phenyl)-6-(1H-pyrazol-5-yl)picolinamide (Compound 081)

Prepared according to the procedure for 061 to give 115.8 mg of the desired product in 15.3% yield. LCMS (m/z): 554.1 [M+H]⁺. ¹H NMR (400 MHz, DMSO) δ 13.95-13.28 (m, 1H), 10.99-10.86 (m, 1H), 8.98-7.56 (m, 7H), 7.11-6.79 (m, 2H), 6.11 (dd, J=16.4, 8.4 Hz, 1H), 5.71 (s, 1H), 4.75-3.69 (m, 3H), 3.49-3.34 (m, 2H), 2.29-2.15 (m, 1H), 2.08-1.66 (m, 2H), 1.61-1.49 (m, 1H).

### (S)-N-(4-(5-(1-acryloylpiperidin-3-yl)-1,2,4-oxadiazol-3-yl)-2-methoxyphenyl)-6-(1H-pyrazol-5-yl)picolinamide (Compound 084)

Prepared according to the procedure for 061 to give 34.6 mg of the desired product in 15.5% yield. LCMS (m/z): 500.2 [M+H]⁺. ¹H NMR (400 MHz, DMSO-d₆) δ 13.92-13.26 (m, 1H), 10.94-10.73 (m, 1H), 8.63 (d, J=8.4 Hz, 1H), 8.31-7.92 (m, 4H), 7.75-7.62 (m, 2H), 7.07-6.80 (m, 2H), 6.12 (dd, J=16.8, 9.6 Hz, 1H), 5.71 (d, J=10.8 Hz, 1H), 4.65-3.76 (m, 6H), 3.46-3.34 (m, 2H), 2.30-2.14 (m, 1H), 2.06-1.76 (m, 2H), 1.61-1.50 (m, 1H).

### (S)-N-(4-(5-(1-acryloylpiperidin-3-yl)-1,2,4-oxadiazol-3-yl)phenyl)-6-(4-methyl-1H-pyrazol-5-yl)picolinamide (Compound 085)

Prepared according to the procedure for 061 to give 63.9 mg of the desired product in 56.8% yield. LCMS (m/z): 484.1 [M+H]⁺. ¹H NMR (400 MHz, DMSO-d₆) δ 13.84-12.96 (m, 1H), 10.91-10.51 (m, 1H), 8.28-7.48 (m, 8H), 6.97-6.78 (m, 1H), 6.11 (t, J=14.8 Hz, 1H), 5.70 (d, J=10.4 Hz, 1H), 4.67-4.12 (m, 1H), 4.03-3.17 (m, 4H), 2.68-2.51 (m, 1H), 2.39 (s, 2H), 2.27-2.14 (m, 1H), 2.05-1.69 (m, 2H), 1.63-1.50 (m, 1H).

### (S)-N-(4-(5-(1-acryloylpiperidin-3-yl)-1,2,4-oxadiazol-3-yl)phenyl)-6-(4-chloro-1H-pyrazol-5-yl)picolinamide (Compound 086)

Prepared according to the procedure for 061 to give 66.8 mg of the desired product in 49.7% yield. LCMS (m/z): 504.2 [M+H]⁺. ¹H NMR (400 MHz, DMSO-d₆) δ 14.38 (s, 1H), 10.90 (s, 1H), 8.48-7.81 (m, 8H), 6.99-6.78 (m, 1H), 6.11 (t, J=15.2 Hz, 1H), 5.70 (d, J=10.0 Hz, 1H), 4.63-4.11 (m, 1H), 4.05-3.41 (m, 2H), 3.29-3.23 (m, 2H), 2.29-2.16 (m, 1H), 2.05-1.67 (m, 2H), 1.62-1.52 (m, 1H).

### (S)-N-(4-(5-(1-acryloylpiperidin-3-yl)-1,2,4-oxadiazol-3-yl)-2-fluorophenyl)-6-(4-methyl-1H-pyrazol-5-yl)picolinamide (Compound 088)

Prepared according to the procedure for 061 to give 119.5 mg of the desired product in 46.4% yield. LCMS (m/z): 502.0 [M+H]⁺. ¹H NMR (400 MHz, CDCl₃) δ 10.52 (s, 1H), 8.80 (t, J=8.0 Hz, 1H), 8.28-8.12 (m, 2H), 8.03-7.91 (m, 2H), 7.87 (d, J=11.6 Hz, 1H), 7.52 (s, 1H), 6.84-6.54 (m, 1H), 6.32 (d, J=16.8 Hz, 1H), 5.74 (d, J=10.8 Hz, 1H), 5.15-3.01 (m, 5H), 2.57 (s, 3H), 2.33 (s, 1H), 2.15-1.71 (m, 3H).

### (S)-N-(4-(5-(1-acryloylpiperidin-3-yl)-1,2,4-oxadiazol-3-yl)-2-fluorophenyl)-6-(4-chloro-1H-pyrazol-5-yl)picolinamide (Compound 089)

Prepared according to the procedure for 061 to give 18.1 mg of the desired product in 27% yield. LCMS (m/z): 522.1 [M+H]⁺. ¹H NMR (400 MHz, DMSO) δ 13.13 (s, 1H), 10.79 (s, 1H), 8.52-7.85 (m, 7H), 6.99-6.77 (m, 1H), 6.16-6.06 (m, 1H), 5.70 (d, J=10.4 Hz, 1H), 4.55-3.32 (m, 5H), 2.28-2.15 (m, 1H), 2.05-1.70 (m, 2H), 1.61-1.49 (m, 1H).

### (S)-N-(4-(5-(1-acryloyl-5,5-difluoropiperidin-3-yl)-1,2,4-oxadiazol-3-yl)-2-fluorophenyl)-6-(1H-pyrazol-5-yl)picolinamide (Compound 090)

Prepared according to the procedure for 061 to give 68.1 mg of the desired product in 44.3% yield. LCMS (m/z): 524.1 [M+H]⁺. ¹H NMR (400 MHz, DMSO-d₆) δ 13.64 (s, 1H), 10.85 (s, 1H), 8.45-7.64 (m, 7H), 7.14-6.82 (m, 2H), 6.22 (d, J=16.4 Hz, 1H), 5.82 (t, J=12.0 Hz, 1H), 4.87-3.27 (m, 5H), 2.84-2.56 (m, 2H).

### N-(4-(5-(1-acryloyl-5,5-difluoropiperidin-3-yl)-1,2,4-oxadiazol-3-yl)-2-fluorophenyl)-6-(4-chloro-1H-pyrazol-5-yl)picolinamide (Compound 092)

Prepared according to the procedure for 061 to give 36.2 mg of the desired product in 41.4% yield. LCMS (m/z): 558.0 [M+H]⁺. ¹H NMR (400 MHz, DMSO-d₆) δ 13.95 (s, 1H), 10.80 (s, 1H), 8.55-7.89 (m, 7H), 7.12-6.86 (m, 1H), 6.21 (d, J=16.8 Hz, 1H), 5.82 (t, J=12.0 Hz, 1H), 4.90-3.16 (m, 5H), 2.85-2.59 (m, 2H).

### N-(4-(5-(1-acryloyl-5,5-difluoropiperidin-3-yl)-1,2,4-oxadiazol-3-yl)-2-fluorophenyl)-6-(4-methyl-1H-pyrazol-5-yl)picolinamide (Compound 093)

Prepared according to the procedure for 061 to give 51.6 mg of the desired product in 27% yield. LCMS (m/z): 538.0 [M+H]⁺. ¹H NMR (400 MHz, DMSO-d₆) δ 13.71-13.01 (m, 1H), 10.83-10.52 (m, 1H), 8.85-7.44 (m, 7H), 7.13-6.86 (m, 1H), 6.22 (dd, J=16.8, 5.6 Hz, 1H), 5.82 (t, J=12.0 Hz, 1H), 4.82-3.23 (m, 7H), 2.89-2.54 (m, 3H).

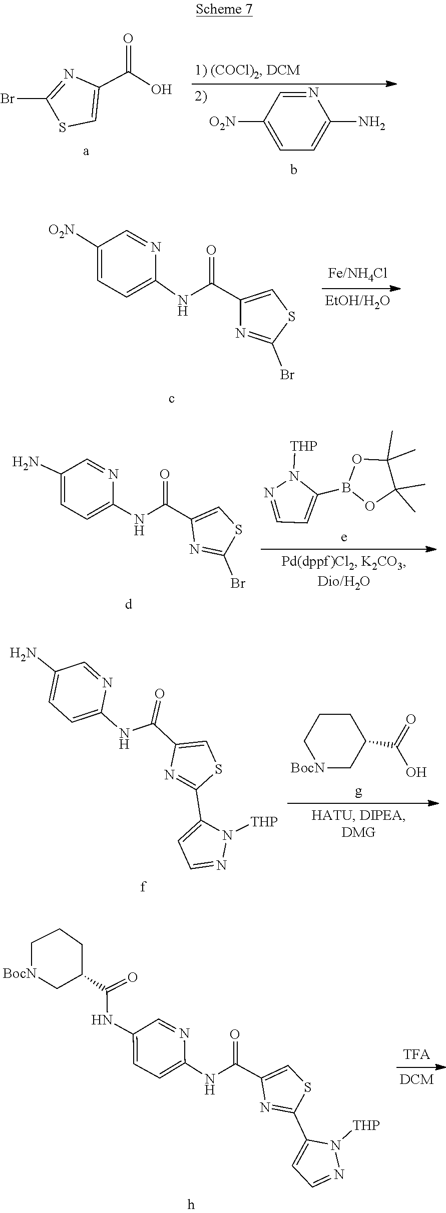

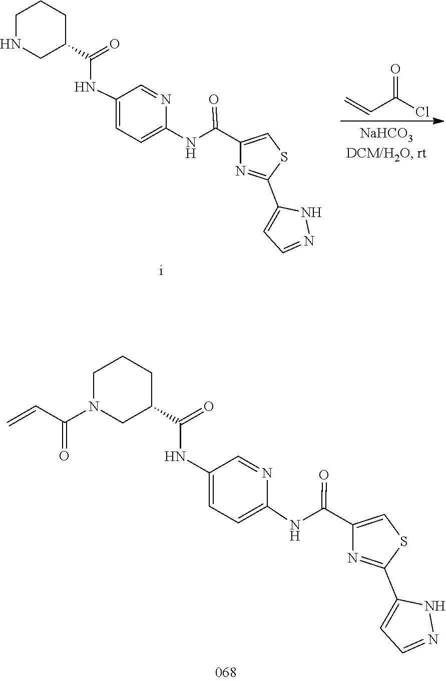

### Synthesis of 2-bromo-N-(5-nitropyridin-2-yl)thiazole-4-carboxamide(c)

To a solution of 2-bromothiazole-4-carboxylic acid (1 g, 4.8 mmol) in DCM (10 mL) and DMF (2 drops) was added oxalyl chloride (915 mg, 7.2 mmol) at 0°C, the mixture was stirred at 0°C for 15 min, and then warmed to room temperature. The mixture was stirred at room temperature for 2 hs, and then concentrated. The residue was dissolved with DCM (20 mL), and this solution was added dropwisely to a solution of 5-nitropyridin-2-amine (738 mg, 5.3 mmol) and Et3N (1.4 g, 13.2 mmol) in DCM (20 mL) at 0°C, and the resulting mixture was stirred at room temperature overnight. The mixture was diluted with DCM (20 mL), and the organic phase was washed with water (30 mL), brine (30 mL) dried over anhydrous Na₂SO₄ and concentrated. The residue was purified by flash column chromatography on silica gel (eluting with petroleum ether/EtOAc=20/1-5/1) to afford compound b as pale solid. (760 mg, 52% isolated yield). LCMS (m/z): 328.9 [M+H]⁺.

### Synthesis of N-(5-aminopyridin-2-yl)-2-bromothiazole-4-carboxamide (d)

To a solution of 2-bromo-N-(5-nitropyridin-2-yl)thiazole-4-carboxamide (760 mg, 3.23 mmol) and NH₄Cl (985 mg 18.59 mmol) in EtOH/H2O (20 mL/10 mL) was added Fe (520 mg, 9.29 mmol) at 80°C, and filtered through the celite. The filtrate was concentrated, the residue was purified by flash column chromatography on silica gel (eluting with petroleum ether/EtOAc=20/1-4/1) to afford compound d as pale solid. (300 mg, 43% isolated yield). LCMS (m/z): 298.9 [M+H]⁺.

### Synthesis of N-(5-aminopyridin-2-yl)-2-(1-(tetrahydro-2H-pyran-2-yl)-1H-pyrazol-5-yl)thiazole-4-carboxamide (f)

To a solution of N-(5-aminopyridin-2-yl)-2-bromothiazole-4-carboxamide (300 mg, 1 mmol) in 1,4-dioxane/water (20/5 mL) was added 1-(tetrahydro-2H-pyran-2-yl)-5-(4,4,5,5-tetramethyl-1,3,2-dioxaborolan-2-yl)-1H-pyrazole (389 mg, 1.2 mmol), Pd(dppf)Cl₂ (73 mg, 0.1 mmol) and K₂CO₃ (292 mg, 2 mmol). The resulting mixture was stirred at 100°C under nitrogen atmosphere for overnight and concentrated. The residue was purified by flash chromatography (eluting with petroleum ether/EtOAc=20/1-4/1) to afford compound f as pale solid. (150 mg, 40% isolated yield). LCMS (m/z): 317.0 [M+H]⁺.

### Synthesis of tert-butyl (3S)-3-((6-(2-(1-(tetrahydro-2H-pyran-2-yl)-1H-pyrazol-5-yl)thiazole-4-carboxamido)pyridin-3-yl)carbamoyl)piperidine-1-carboxylate (h)

To a solution of N-(5-aminopyridin-2-yl)-2-(1-(tetrahydro-2H-pyran-2-yl)-1H-pyrazol-5-yl)thiazole-4-carboxamide (150 mg, 0.41 mmol) in DMF (15 mL) was added (S)-1-(tert-butoxycarbonyl)piperidine-3-carboxylic acid (111 mg, 0.48 mmol), HATU (231 g, 0.61 mmol) and DIPEA (79 mg, 0.61 mmol). The resulting mixture was stirred at room temperature overnight, and quenched with water (50 mL), the aqueous phase was extracted with EtOAc (40 mL*3). The combined organic phase was concentrated, the residue was purified by flash chromatography (eluting with petroleum ether/EtOAc=20/1-2/1) to afford compound h as brown solid. (200 mg, 85% isolated yield). LCMS (m/z): 582.2 [M+H]⁺.

### Synthesis of (S)-N-(5-(piperidine-3-carboxamido)pyridin-2-yl)-2-(1H-pyrazol-5-yl)thiazole-4-carboxamide (i)

To a solution of tert-butyl (3S)-3-((6-(2-(1-(tetrahydro-2H-pyran-2-yl)-1H-pyrazol-5-yl)thiazole-4-carboxamido)pyridin-3-yl)carbamoyl)piperidine-1-carboxylate (200 mg, 0.34 mmol) in DCM (5 mL) was added TFA (1 mL) at 0°C. The resulting mixture was stirred at room temperature for 16 hours, and then diluted with DCM (30 mL). The organic phase was washed with aq. sat. Na₂CO₃ (15 mL×2), water (20 mL) and dried over anhydrous Na₂SO₄ and concentrated to afford compound i as brown oil. (100 mg, 73% isolated yield). LCMS (m/z): 398.2 [M+H]⁺.

### Synthesis of (S)-N-(5-(1-acryloylpiperidine-3-carboxamido)pyridin-2-yl)-2-(1H-pyrazol-5-yl)thiazole-4-carboxamide (Compound 068)

To a solution of (S)-N-(5-(piperidine-3-carboxamido)pyridin-2-yl)-2-(1H-pyrazol-5-yl)thiazole-4-carboxamide (100 mg, 0.25 mmol) in DCM (4 mL) and 5% NaHCO₃ (1.0 mL) was added a solution of acryloyl chloride (27 mg, 0.31 mmol) in DCM (1 mL) at 0°C, the mixture was stirred at 0°C for 1 hour. The resulting mixture was diluted with water (10 mL), and the aqueous phase was extracted with DCM (10 mL×3). The combined organic was concentrated, the residue was purified by Prep-HPLC to afford compound DFCI-001-3 as white solid. (45 mg, 40% isolated yield). LCMS (m/z): 452.1 [M+H]⁺. ¹H NMR (400 MHz, MeOD-d₄) δ 8.76 (d, J=13.2 Hz, 1H), 8.41 (s, 1H), 8.17 (s, 2H), 7.80 (d, J=2.4 Hz, 1H), 7.00 (d, J=2.0 Hz, 1H), 6.90-6.69 (m, 1H), 6.21 (d, J=16.8 Hz, 1H), 5.84-5.66 (m, 1H), 4.61-4.37 (m, 1H), 4.21-4.05 (m, 1H), 3.53-3.23 (m, 1H), 3.15-2.86 (m, 1H), 2.66-2.52 (m, 1H), 2.18-2.04 (m, 1H), 1.94-1.79 (m, 2H), 1.65-1.48 (m, 1H).

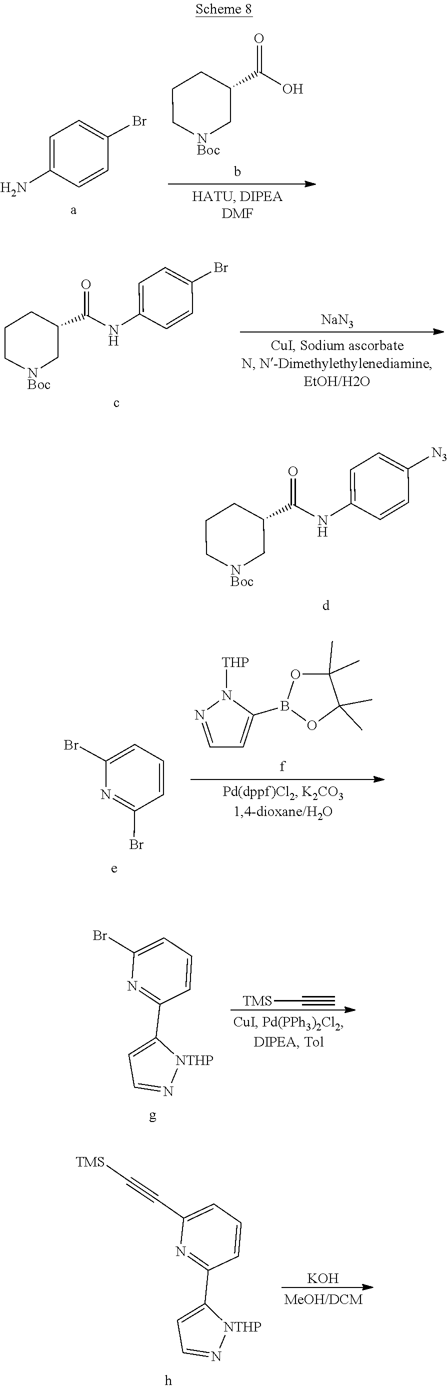

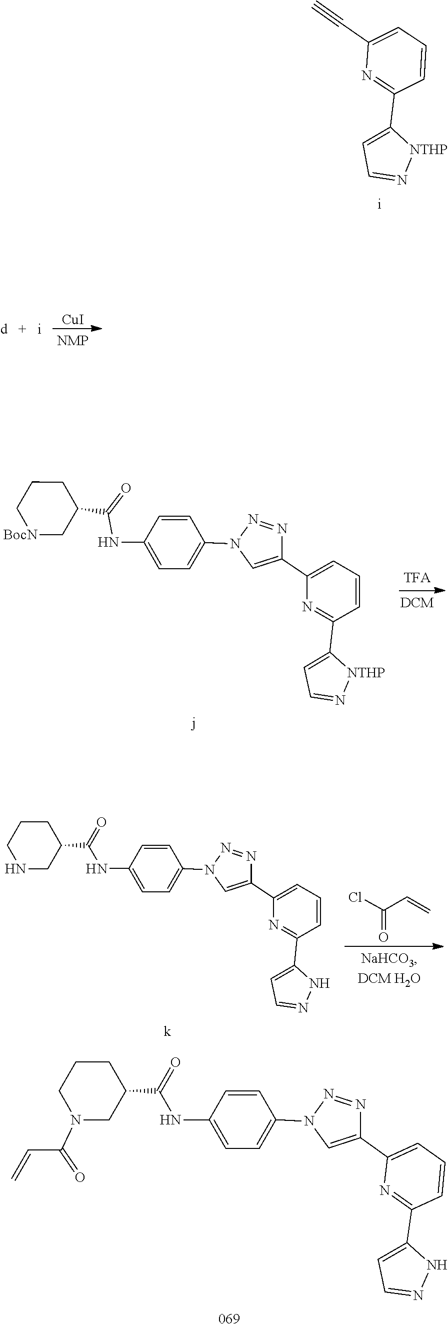

### Synthesis of tert-butyl (S)-3-((4-bromophenyl)carbamoyl)piperidine-1-carboxylate (c)

To a solution of 4-bromoaniline (860 mg, 5.06 mmol) and (S)-1-(tert-butoxycarbonyl)piperidine-3-carboxylic acid (1.39 g, 6.07 mmol) in DMF (5 mL) was added HATU (2.88 g, 7.59 mmol) and DIPEA (1.31 g, 10.1 mmol). The reaction mixture was stirred at room temperature for 16 hours, diluted with water (50 mL). The aqueous phase was extracted with EtOAc (30 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/1) to afford compound c as a light yellow oil. (1.8 g, 93% isolated yield). LCMS (m/z): 327.0 [M+H−56]⁺.

### Synthesis of tert-butyl (S)-3-((4-azidophenyl)carbamoyl)piperidine-1-carboxylate (d)

To a solution of tert-butyl (S)-3-((4-bromophenyl)carbamoyl)piperidine-1-carboxylate (400 mg, 1.05 mmol), sodium azide (123 mg, 1.88 mmol), sodium ascorbate (10 mg, 0.05 mmol,) and N, N′-dimethylethylenediamine (14 mg, 0.16 mmol) in EtOH/H₂O(7/3, 10 mL) was added Cul (20 mg, 0.10 mmol). The reaction mixture was heated for 1.5 hours under nitrogen atmosphere in a Boitage Initiator Eight Microwave Reactor at a constant temperature of 100°C. The resulting mixture was diluted with water (30 mL), the aqeous phase was extracted with EtOAc (30 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/1) to afford compound d as a pale solid. (130 mg, 36% isolated yield).LCMS (m/z): 290.1 [M+H-56]⁺.

### Synthesis of 2-bromo-6-(1-(tetrahydro-2H-pyran-2-yl)-1H-pyrazol-5-yl)pyridine (g)

To a solution of 2,6-dibromopyridine (2.35 g, 10 mmol) in 1,4-dioxane/water (20 mL/5 mL) was added 1-(tetrahydro-2H-pyran-2-yl)-5-(4,4,5,5-tetramethyl-1,3,2-dioxaborolan-2-yl)-1H-pyrazole (1.81 g, 6.5 mmol), Pd(dppf)Cl₂ (366 mg, 0.5 mmol) and K₂CO₃ (2.76 g, 20 mmol). The resulting mixture was stirred at 95°C overnight under nitrogen atmosphere and concentrated. The residue was purified by flash chromatography (eluting with dichloromethane: methanol=0-68%) to give compound g (998 mg, yellow oil, yield 32.5%). LCMS (m/z): 598.2 [M+H]⁺.

### Synthesis of 2-(1-(tetrahydro-2H-pyran-2-yl)-1H-pyrazol-5-yl)-6-((trimethylsilyl)ethynyl)pyridine (h)

A mixture of 2-bromo-6-(1-(tetrahydro-2H-pyran-2-yl)-1H-pyrazol-5-yl)pyridine (998 mg, 3.25 mmol), Pd(PPh₃)₂Cl₂ (57.1 g, 0.08 mmol) and Cul (15.2 mg, 0.08 mmol) in Tol (10 mL) was stirred at room temperature under nitrogen atmosphere for 10 min. Ethynyltrimethylsilane (382.3 mg, 3.90 mmol) was added and stirred at room temperature for 5 min, then warming to 40°C DIPEA (10 mL) was added, the resulting mixture was stirred at 40°C under nitrogen atmosphere for 2 hs and then queched with water (20 mL). The aqueous phase was exratcted with ethyl acetate (15 mL×3), and the combined organic phase was concentrated, the resiude was purified by purified by flash chromatography (eluting with petrol ether: ethyl acetate=0-35%) to give compound h (1 g, yellow oil, yield 94.6%). LCMS (m/z): 326.1 [M+H]⁺.

### Synthesis of 2-ethynyl-6-(1-(tetrahydro-2H-pyran-2-yl)-1H-pyrazol-5-yl)pyridine (i)

To a solution of 2-(1-(tetrahydro-2H-pyran-2-yl)-1H-pyrazol-5-yl)-6-((trimethylsilyl)ethynyl)pyridine (1 g, 3.08 mmol) in MeOH/DCM (12 mL/6 mL) was added KOH (344.6 mg, 6.15 mmol), and the resulting mixture was stirred at room temperature overnight and concentrated. The residue was added water (20 mL) and DCM (15 mL), the organic phase was sepreated and the aqueous phase was exratcted with DCM (15 mL×2). The combined organic phase was concentrated, the residue was purified by flash chromatography (eluting with petrol ether: ethyl acetate=0-48%) to give compound i (500 mg, colorless oil, yield 64.3%). LCMS (m/z): 326.1 [M+H]⁺.

### Synthesis of tert-butyl (3S)-3-((6-(4-(6-(1-(tetrahydro-2H-pyran-2-yl)-1H-pyrazol-5-yl)pyridin-2-yl)-1H-1,2,3-triazol-1-yl)pyridin-3-yl)carbamoyl)piperidine-1-carboxylate (j)

Toa solution of tert-butyl (S)-3-((4-azidophenyl)carbamoyl)piperidine-1-carboxylate (130 mg, 0.38 mmol) and 2-ethynyl-6-(1-(tetrahydro-2H-pyran-2-yl)-1H-pyrazol-5-yl)pyridine (114 mg, 0.45 mmol) in NMP (5 mL) was added Cul (107 mg, 0.57 mmol), the reaction mixture was stirred at 80°C for 16 hours under nitrogen atmosphere. The resulting mixture was diluted with water (30 mL), the aqeous phase was extracted with EtOAc (30 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/1) to afford compound j as a pale solid. (120 mg, 53% isolated yield). LCMS (m/z): 599.2 [M+H]⁺.

### Synthesis of (S)-N-(4-(4-(6-(1H-pyrazol-5-yl)pyridin-2-yl)-1H-1,2,3-triazol-1-yl)phenyl)piperidine-3-carboxamide (k)

To a solution of tert-butyl (3S)-3-((6-(4-(6-(1-(tetrahydro-2H-pyran-2-yl)-1H-pyrazol-5-yl)pyridin-2-yl)-1H-1,2,3-triazol-1-yl)pyridin-3-yl)carbamoyl)piperidine-1-carboxylate (120 mg, 8.8 mmol) in DCM (5 mL) was added TFA (1 mL) at 0°C. The resulting mixture was stirred at room temperature for 16 hours, and then diluted with DCM (30 mL). The organic phase was washed with aq. sat. Na₂CO₃ (15 mL×2), water (20 mL) and dried over anhydrous Na₂SO₄ and concentrated to afford compound k as brown oil (120 mg), which was used directly for the next step. LCMS (m/z): 415.1 [M+H]⁺.

### Synthesis of (S)-N-(4-(4-(6-(1H-pyrazol-5-yl)pyridin-2-yl)-1H-1,2,3-triazol-1-yl)phenyl)-1-acryloylpiperidine-3-carboxamide (Compound 069)

To a solution of (S)-N-(4-(4-(6-(1H-pyrazol-5-yl)pyridin-2-yl)-1H-1,2,3-triazol-1-yl)phenyl)piperidine-3-carboxamide (120 mg, crude) in DCM (4 mL) and 5% NaHCO₃ (1.0 mL) was added a solution of acryloyl chloride (22 mg, 0.24 mmol) in DCM (1 mL) at 0°C, the mixture was stirred at 0°C for 1 hour. The resulting mixture was diluted with water (10 mL), and the aqueous phase was extracted with DCM (10 mL×3). The combined organic was concentrated, the residue was purified by Prep-HPLC to give compound Compound 069 (10 mg, two steps yield 11%) as white solid. LCMS (m/z): 469.1 [M+H]⁺. ¹H NMR (400 MHz, MeOD-d₄) δ 9.20 (s, 1H), 8.11-7.56 (m, 8H), 7.08-6.93 (m, 1H), 6.89-6.77 (m, 1H), 6.21 (dd, J=16.8, 4.4 Hz, 1H), 5.83-5.67 (m, 1H), 4.73-4.41 (m, 1H), 4.23-4.07 (m, 1H), 3.54-3.18 (m, 1H), 3.06-2.87 (m, 1H), 2.66-2.53 (m, 1H), 2.17-2.01 (m, 1H), 1.96-1.83 (m, 2H), 1.67-1.49 (m, 1H).

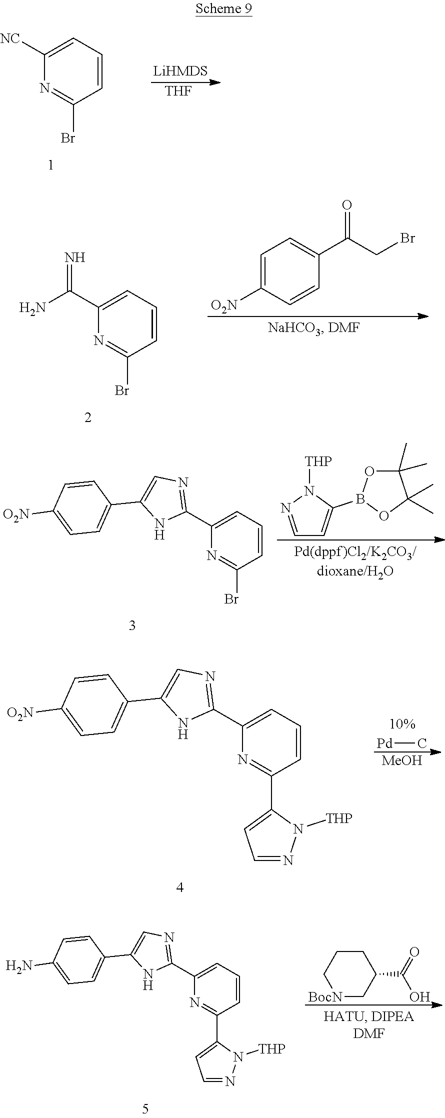

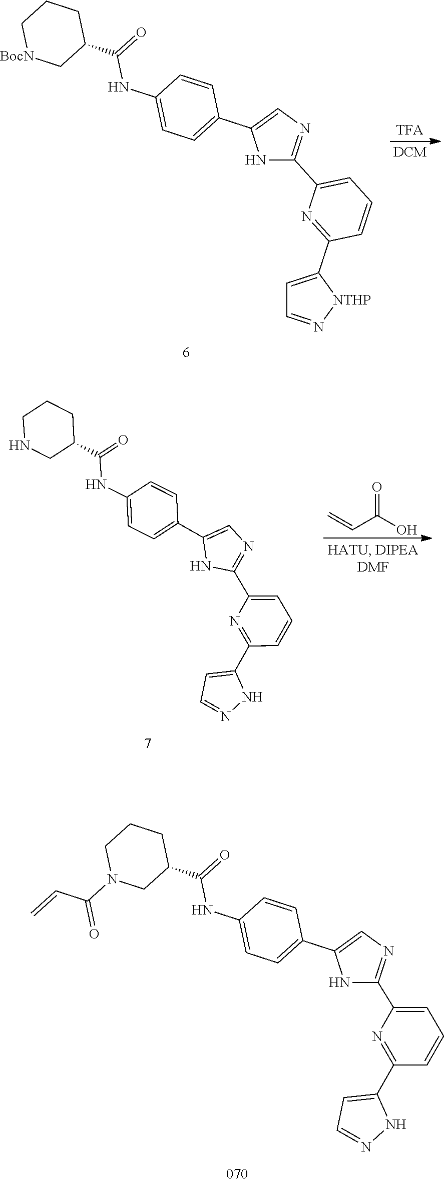

### Synthesis of 6-bromopicolinimidamide (2)

To a solution of 6-bromopicolinonitrile (2 g, 10.99 mmol) in THE (30 mL) at −78°C was added dropwisely LiHMDS (1M, 33 mL) and the mixture was stirred at room temperature overnight. The resulting mixture was quenched with water (10 mL) at 0°C, and the aqueous phase was extracted by ethyl acetate (30 mL×3). The combined organic phase was dried by Na₂SO₄, filtered and concentrated to give compound 2 (1.78 g, white solid, yield 81.4%), which was used directly for the next step. LCMS (m/z): 200.0 [M+H]⁺.

### Synthesis of 2-bromo-6-(5-(4-nitrophenyl)-1H-imidazol-2-yl)pyridine (3)

To a solution of 6-bromopicolinimidamide (1.78 g, 8.94 mmol) in DMF (25 mL) was added 2-bromo-1-(4-nitrophenyl)ethan-1-one (2.61 g, 10.73 mmol) and NaHCO₃ (1.50 g, 17.89 mmol). The resulting mixture was stirred at 80°C overnight, and then diluted with water (30 mL), and the aqueous phase was extracted by ethyl acetate (30 mL×3). The combined organic phase was washed by water (30 mL×3) and concentrated, the residue was purified by flash chromatography (eluting with petroleum ether: ethyl acetate=0-30%) to give compound 3 (730 mg, yellow solid, yield 23.7%). LCMS (m/z): 345.0 [M+H]⁺.

### Synthesis of 2-(5-(4-nitrophenyl)-1H-imidazol-2-yl)-6-(1-(tetrahydro-2H-pyran-2-yl)-1H-pyrazol-5-yl)pyridine (4)

To a solution of 2-bromo-6-(5-(4-nitrophenyl)-1H-imidazol-2-yl)pyridine (460 mg, 1.34 mmol) in 1,4-dioxane/water (20 mL/5 mL) was added 1-(tetrahydro-2H-pyran-2-yl)-5-(4,4,5,5-tetramethyl-1,3,2-dioxaborolan-2-yl)-1H-pyrazole (558 mg, 2.00 mmol), Pd(dppf)Cl₂ (48.9 mg, 0.07 mmol) and K₂CO₃ (332 mg, 2.41 mmol). The resulting mixture was stirred at 95°C overnight under nitrogen atmosphere, and then concentrated. The residue was purified by flash chromatography (eluting with petroleum ether: ethyl acetate=0-50%) to give compound 4 (210 mg, red-brown solid, yield 37.7%). LCMS (m/z): 417.1 [M+H]⁺.

### Synthesis of 4-(2-(6-(1-(tetrahydro-2H-pyran-2-yl)-1H-pyrazol-5-yl)pyridin-2-yl)-1H-imidazol-5-yl)aniline (5)

To a solution 2-(5-(4-nitrophenyl)-1H-imidazol-2-yl)-6-(1-(tetrahydro-2H-pyran-2-yl)-1H-pyrazol-5-yl)pyridine (205 mg, 0.49 mmol) in methanol (10 mL) was added 10% Pd-C(200 mg) and the mixture was stirred at room temperature under H₂ atmosphere for 2 hs. The mixture was filtered through the celite, the filtrate was cocnentrated and the residue was purified by flash chromatography (eluting with dichloromethane: methanol=0-25%) to give compound 5 (180 mg, yellow oil, yield 94.6%). LCMS (m/z): 387.2 [M+H]⁺.

### Synthesis of tert-butyl (3S)-3-((4-(2-(6-(1-(tetrahydro-2H-pyran-2-yl)-1H-pyrazol-5-yl)pyridin-2-yl)-1H-imidazol-5-yl)phenyl)carbamoyl)piperidine-1-carboxylate (6)

To a solution of 4-(2-(6-(1-(tetrahydro-2H-pyran-2-yl)-1H-pyrazol-5-yl)pyridin-2-yl)-1H-imidazol-5-yl)aniline (180 mg, 0.47 mmol) in DMF (8 mL) was added (S)-1-(tert-butoxycarbonyl)piperidine-3-carboxylic acid (128 mg, 0.56 mmol), HATU (266 mg, 0.70 mmol) and DIPEA (90.2 mg, 0.70 mmol). The mixture was stirred at room temperature overnight, and diluted with water (15 mL), the aqueous phase was extracted with ethyl acetate (15 mL×3). The combined organic phase was concentrated and the residue was purified by flash chromatography (eluting with petrol ether: ethyl acetate=0-50%) to give compound 6 (200 mg, yellow oil, yield 71.8%). LCMS (m/z): 598.3 [M+H]⁺.

### Synthesis of (S)-N-(4-(2-(6-(1H-pyrazol-5-yl)pyridin-2-yl)-1H-imidazol-5-yl)phenyl)piperidine-3-carboxamide (7)

To a solution of tert-butyl (3S)-3-((4-(2-(6-(1-(tetrahydro-2H-pyran-2-yl)-1H-pyrazol-5-yl)pyridin-2-yl)-1H-imidazol-5-yl)phenyl)carbamoyl)piperidine-1-carboxylate (200 mg, 0.34 mmol) in dichloromethane (7 mL) was added TFA (4 mL). The resulting mixture was stirred at room temperature for 2 hs and concentrated to give compound 7 (160 mg, crude), which was used directly for the next step. LCMS (m/z): 414.1 [M+H]⁺.

### Synthesis of (S)-N-(4-(2-(6-(1H-pyrazol-5-yl)pyridin-2-yl)-1H-imidazol-5-yl)phenyl)-1-acryloylpiperidine-3-carboxamide (Compound 070)

To a solution of (S)-N-(4-(2-(6-(1H-pyrazol-5-yl)pyridin-2-yl)-1H-imidazol-5-yl)phenyl)piperidine-3-carboxamide (135 mg, crude) in DMF (6 mL) was added acrylic acid (30.6 mg, 0.42 mmol), HATU (186.3 mg, 0.49 mmol) and DIPEA (63.3 mg, 0.49 mmol). The mixture was stirred at room temperature overnight, and diluted with water (15 mL), the aqueous phase was extracted with ethyl acetate (15 mL×3). The combined organic phase was concentrated and the residue was purified by Prep-HPLC to give compound 070 (17.7 mg, white solid, yield 11.3% two steps). LCMS (m/z): 468.1 [M+H]⁺. ¹H NMR (400 MHz, MeOD-d₄) δ 8.12-7.43 (m, 9H), 7.01 (s, 1H), 6.87-6.72 (m, 1H), 6.25-6.15 (m, 1H), 5.75 (dd, J=10.8, 4.4 Hz, 1H), 4.62-4.07 (m, 2H), 3.53-2.81 (m, 2H), 2.65-2.49 (m, 1H), 2.15-2.03 (m, 1H), 1.94-1.82 (m, 2H), 1.59-1.51 (m, 1H).

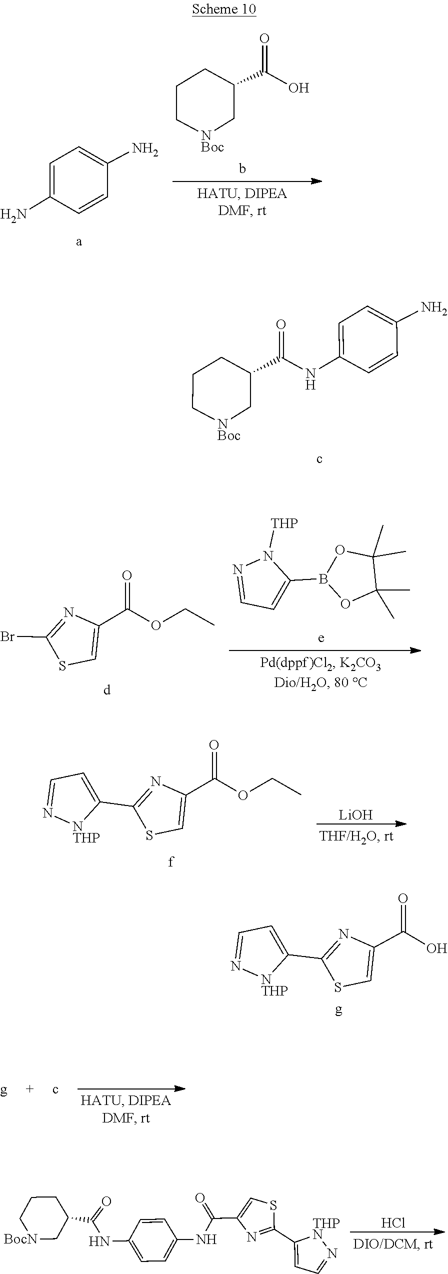

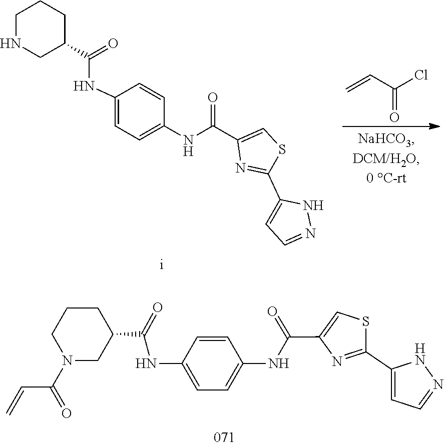

### Synthesis of tert-butyl (S)-3-((4-aminophenyl)carbamoyl)piperidine-1-carboxylate (c)

To a solution of benzene-1,4-diamine (540 mg, 5 mmol) in DMF (15 mL) was added (S)-1-(tert-butoxycarbonyl)piperidine-3-carboxylic acid (954 mg, 4.2 mmol), HATU (2.3 g, 6.3 mmol) and DIPEA (806 mg, 6.3 mmol). The resulting mixture was stirred at room temperature overnight, and quenched with water (30 mL), and the aqueous phase was extracted with EtOAc (20 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/1) to afford compound c as light brown oil. (1.4 g, 87% isolated yield). LCMS (m/z): 264.1 [M+H-Boc]⁺.

### Synthesis of ethyl 2-(1-(tetrahydro-2H-pyran-2-yl)-1H-pyrazol-5-yl)thiazole-4-carboxylate (f)

To a solution of ethyl 2-bromothiazole-4-carboxylate (2.16 g, 10 mmol) in 1,4-dioxane/water (25 mL/5 mL) were added 1-(tetrahydro-2H-pyran-2-yl)-5-(4,4,5,5-tetramethyl-1,3,2-dioxaborolan-2-yl)-1H-pyrazole (2.78 g, 10 mmol), Pd(dppf)Cl₂ (731 mg, 1 mmol) and K₂CO₃(2.76 g, 20 mmol). The resulting mixture was stirred at 95°C overnight under nitrogen atmosphere, and then concentrated. The residue was purified by reverse phase chromatography (eluting with petroleum ether:ethyl acetate=0-20%) to afford compound f as white solid (1 g, 33% isolated yield). LCMS (m/z): 308.1 [M+H]⁺.

### Synthesis of 2-(1-(tetrahydro-2H-pyran-2-yl)-1H-pyrazol-5-yl)thiazole-4-carboxylic acid (g)

To a solution of ethyl 2-(1-(tetrahydro-2H-pyran-2-yl)-1H-pyrazol-5-yl)thiazole-4-carboxylate (1 g, 3.47 mmol) in THE (10 mL) and H₂O (10 mL) was added LiOH·H2O (555 mg, 13.8 mmol). The resulting solution was stirred at room temperature for 2 hs, and then concentrated. The residue was added H₂O (20 mL), and the pH of the solution was adjusted with 1N HCl to 4-5, and then the aqueous phase was extracted with EtOAc (20 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/3) to afford compound g as yellow solid. (700 mg, 78% isolated yield). LCMS (m/z): 280.1 [M+H]⁺.

### Synthesis of tert-butyl (3S)-3-((4-(2-(1-(tetrahydro-2H-pyran-2-yl)-1H-pyrazol-5-yl)thiazole-4-carboxamido)phenyl)carbamoyl)piperidine-1-carboxylate (h)

To a solution of tert-butyl (S)-3-((4-aminophenyl)carbamoyl)piperidine-1-carboxylate (318 mg, 1 mmol) in DMF (5 mL) was added 2-(1-(tetrahydro-2H-pyran-2-yl)-1H-pyrazol-5-yl)thiazole-4-carboxylic acid (280 mg, 1 mmol), HATU (570 mg, 1.5 mmol) and DIPEA (200 mg, 1.5 mmol). The resulting mixture was stirred at room temperature overnight, and quenched with water (30 mL), and the aqueous phase was extracted with EtOAc (20 mL×3), and the combined organic phase was washed with water (50 mL), brine (50 mL) dried over anhydrous Na₂SO₄ and concentrated. The residue was purified by flash column chromatography on silica gel (eluting with petroleum ether/EtOAc=20/1-1/1) to afford compound h as yellow solid. (200 mg, 35% isolated yield). LCMS (m/z): 581.1 [M+H]⁺.

### Synthesis of (S)-N-(4-(piperidine-3-carboxamido)phenyl)-2-(1H-pyrazol-5-yl)thiazole-4-carboxamide (i)

To a solution of tert-butyl (3S)-3-((4-(2-(1-(tetrahydro-2H-pyran-2-yl)-1H-pyrazol-5-yl)thiazole-4-carboxamido)phenyl)carbamoyl)piperidine-1-carboxylate (200 mg, 0.34 mmol) in 1,4-dioxane (5 mL) was added HCl (5 mL, 4 N n 1,4-dioxane). The mixture was stirred at room temperature for 2 hs and concentrated to afford compound i as brown solid (100 mg, crude), which was used directly for the next step. LCMS (m/z): 397.2 [M+H]⁺.

### Synthesis of (S)-N-(4-(1-acryloylpiperidine-3-carboxamido)phenyl)-2-(1H-pyrazol-5-yl)thiazole-4-carboxamide (Compound 071)

To a solution of (S)-N-(4-(piperidine-3-carboxamido)phenyl)-2-(1H-pyrazol-5-yl)thiazole-4-carboxamide (100 mg, crude) and aq. sat. NaHCO₃ (2 mL) in DCM (6 mL) was added a solution of acryloyl chloride (27 mg, 0.30 mmol) in DCM (2 mL). The resulting mixture was stirred at 0°C for 1 h, and diluted with water (10 mL), the aqueous phase was extracted by DCM (15 mL×3). The combined organic phase was concentrated, the residue was purified by Prep-HPLC to afford Compound 071 as white solid. (45 mg, 39% isolated yield of two steps). LCMS (m/z): 451.1 [M+H]⁺. ¹H NMR (400 MHz, CDCl₃) δ 10.82 (s, 1H), 9.20 (s, 1H), 8.89 (s, 1H), 8.17 (s, 1H), 7.71-7.51 (m, 5H), 6.94 (s, 1H), 6.60 (dd, J=16.8, 10.4 Hz, 1H), 6.37 (d, J=16.8 Hz, 1H), 5.77 (d, J=11.2 Hz, 1H), 4.23-3.80 (m, 2H), 3.70-3.50 (m, 2H), 2.94-1.67 (m, 5H).

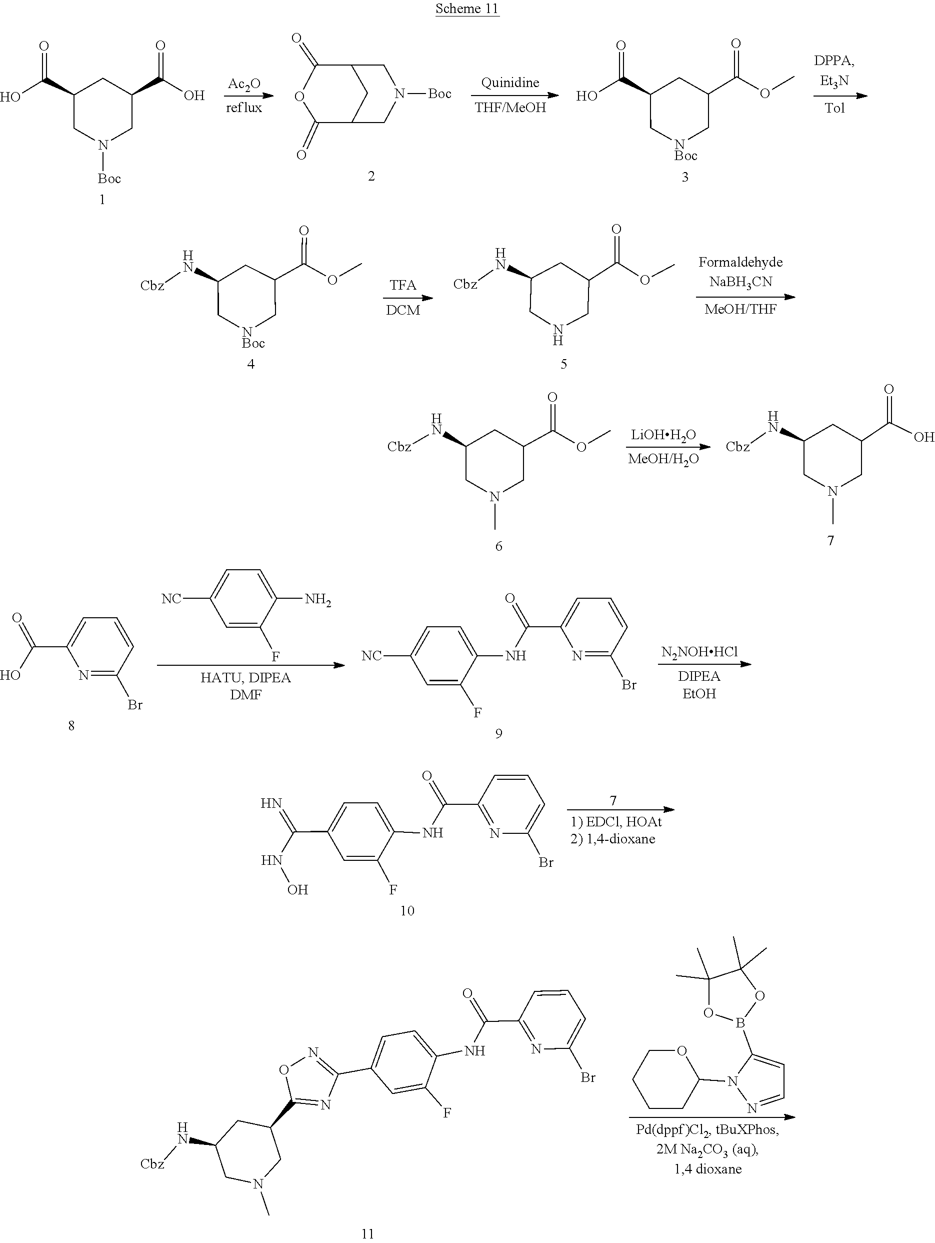

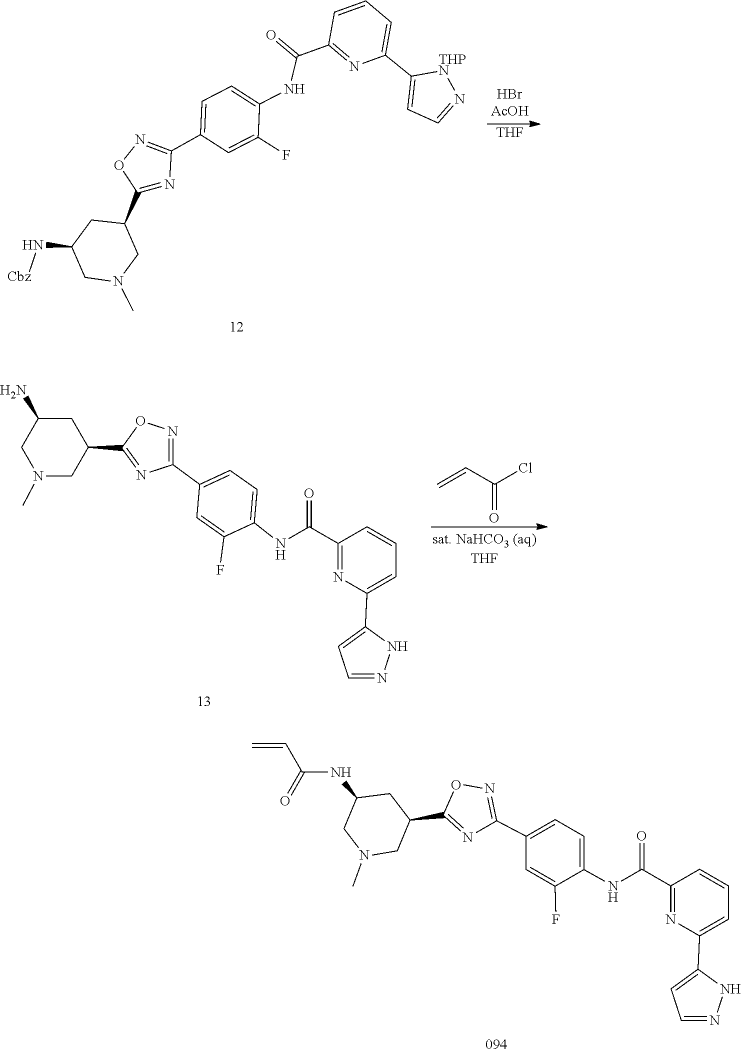

### Synthesis of tert-butyl 2,4-dioxo-3-oxa-7-azabicyclo[3.3.1]nonane-7-carboxylate (2)

A solution of (3S,5R)-1-(tert-butoxycarbonyl)piperidine-3,5-dicarboxylic acid (15.5 g, 56.78 mmol) in Ac₂O (100 mL) was refluxed for 5 hs. The resulting solution was concentrated, the residue was dissolved with toluene (100 mL), and then concentrated to give compound 2 (13.6 g, white solid, crude), which was used directly for the next step. LCMS (m/z): 200.1 [M+H-55]⁺.

### Synthesis of (3S)-1-(tert-butoxycarbonyl)-5-(methoxycarbonyl)piperidine-3-carboxylic acid (3)

A solution of tert-butyl 2,4-dioxo-3-oxa-7-azabicyclo[3.3.1]nonane-7-carboxylate (7.38 g, 28.94 mmol) and quinidine(14.07 g, 43.41 mmol) in THF (70 mL) was stirred at −40°C under nitrogen atmosphere for 10 mins. The resulting solution was added methanol (11 mL in 22 mL THF) drop-wisely at −40°C and stirred at −40°C for 6 hs, and then diluted with HCl (1M, 100 mL). The organic phase was extracted with ethyl acetate (100 mL×3), and the combined organic phase was washed by brine, dried by Na₂SO₄, filtered and concentrated to give compound 3 (6.4 g, white solid, crude), which was used directly for the next step. LCMS (m/z): 232.1 [M+H-55]⁺.

### Synthesis of 1-(tert-butyl) 3-methyl (5S)-5-(((benzyloxy)carbonyl)amino)piperidine-1,3-dicarboxylate (4)

To a solution of (3S)-1-(tert-butoxycarbonyl)-5-(methoxycarbonyl)piperidine-3-carboxylic acid (4 g, 13.94 mmol) in toluene (30 mL) was added DPPA (4.6 g, 16.72 mmol) and Et₃N (2.12 g, 21.0 mmol). The resulting solution was stirred at 100°C under nitrogen atmosphere for 3 hs, and then added benzy alcohol (1.81 g, 16.72 mmol) and stirred at 100°C for 2 hs. The resulting solution was concentrated, the residue was purified by flash chromatography (eluing with petroleum ether: ethyl acetate=0-30%) to give compound 4 (4.4 g, white solid, yield 69.2%). LCMS (m/z): 293.1 [M+H-100]⁺.

### Synthesis of methyl (5S)-5-(((benzyloxy)carbonyl)amino)piperidine-3-carboxylate (5)

To a solution of 1-(tert-butyl) 3-methyl (5S)-5-(((benzyloxy)carbonyl)amino)piperidine-1,3-dicarboxylate (4.4 g, 11.22 mmol) in dichloromethane (20 mL) was added TFA(10 mL), and the resulting solution was stirred at room temperature for 3 hs and concentrated. The residue was purified by flash chromatography (eluting with dichloromethane: methanol=0-7%) to give compound 5 (3.4 g, yellow oil, yield 88.8%). LCMS (m/z): 293.1 [M+H]⁺.

### Synthesis of methyl (5S)-5-(((benzyloxy)carbonyl)amino)-1-methylpiperidine-3-carboxylate (6)

To a solution of methyl (5S)-5-(((benzyloxy)carbonyl)amino)piperidine-3-carboxylate (3.4 g, 11.64 mmol) in MeOH/THF (1:1, 40 mL) was added 38% HCHO (1.84 g, 23.29 mmol) and NaBH₃CN (1.1 g, 17.47 mmol). The reaction solution was stirred at room temperature for 3 hs and concentrated, the residue was purified by flash chromatography (eluting with dichloromethane: methanol=0-20%) to give compound 6 (2.68 g, yellow oil, yield 75.2%). LCMS (m/z): 307.0 [M+H]⁺.

### Synthesis of (5S)-5-(((benzyloxy)carbonyl)amino)-1-methylpiperidine-3-carboxylic acid (7)

To a solution of (5S)-5-(((benzyloxy)carbonyl)amino)-1-methylpiperidine-3-carboxylate (2.68 g, 8.76 mmol) in methanol (20 mL) was added a solution of LiOH H₂O (736 mg, 17.52 mmol) in H₂O (5 mL), the resulting solution was stirred at room temperature for 3 hs and concentrated. The residue was dissolved with H₂O (30 mL), and pH of the solution was adjusted to 4-5 with 1N HCl, and the aqueous phase was extracted with ethyl acetate (30 mL×3). The combined organic phase was washed by brine, dried by Na₂SO₄, filtered and concentrated, the residue was purified by flash chromatography (eluting with dichloromethane: methanol=0-20%) to give compound 7 (2.54 g, yellow oil, yield 99.3%). LCMS (m/z): 293.1 [M+H]⁺.

### Synthesis of 6-bromo-N-(4-cyano-2-fluorophenyl)picolinamide (9)

To a solution of 6-bromopicolinic acid (5 g, 24.88 mmol) in DMF (50 mL) was added 4-amino-3-fluorobenzonitrile (4.4 g, 32.34 mmol), HATU (17 g, 44.78 mmol) and DIPEA (5.78 g, 44.78 mmol). The resulting mixture was stirred at room temperature overnight and diluted with water (100 mL), and the aqueous phase was extracted with dichloromethane (80 mL×3). The combined organic phase was concentrated, the residue was purified by flash chromatography (eluting with dichloromethane: methanol=0-5%) to give compound 9 (4.77 g, yellow oil, yield 60.1%).LCMS (m/z): 319.9 [M+H]⁺.

### Synthesis of 6-bromo-N-(2-fluoro-4-(N-hydroxycarbamimidoyl)phenyl)picolinamide (10)

To a solution of 6-bromo-N-(4-cyano-2-fluorophenyl)picolinamide (3.7 g, 11.6 mmol) in ethanol (40 mL) was added hydroxylamine hydrochloride (2.4 g, 34.8 mmol) and DIPEA (4.49 g, 34.8 mmol). The resulting mixture was stirred at 80°C overnight, and then diluted with aq. sat. NH₄Cl (80 mL), and the aqueous phase was extracted with ethyl acetate (50 mL×3). The combined organic phase was washed with brine, dried by Na₂SO₄ and concentrated, the residue was purified by flash chromatography (eluting with dichlorometahne: methanol=0-100%) to give compound 10 (3 g, white solid, yield 73.5%). LCMS (m/z): 352.9 [M+H]⁺.

### Synthesis of benzyl ((3S,5R)-5-(3-(4-(6-bromopicolinamido)-3-fluorophenyl)-1,2,4-oxadiazol-5-yl)-1-methylpiperidin-3-yl)carbamate (11)

To a solution of 6-bromo-N-(2-fluoro-4-(N-hydroxycarbamimidoyl)phenyl)picolinamide (2.0 g, 5.68 mmol) in THE (30 mL) was added (5S)-5-(((benzyloxy)carbonyl)amino)-1-methylpiperidine-3-carboxylic acid (1.66 g, 5.68 mmol), EDCI (3.26 g, 17.1 mmol) and HOBt (1.53 g, 11.4 mmol) and the mixture was stirred at room temperature under nitrogen atmosphere overnight. The clear liquid was filtered and concentrated, the residue was dissolved with 1,4-dioxane (20 mL). The mixture was stirred at 80°C overnight and concentrated, the residue was purified by flash chromatography (eluting with dichloromethane: methanol=0-20%) to give compound 11 (530 mg, yellow solid, yield 15.4%). LCMS (m/z): 608.9 [M+H]⁺.

### Synthesis of benzyl ((3S,5R)-5-(3-(3-fluoro-4-(6-(1-(tetrahydro-2H-pyran-2-yl)-1H-pyrazol-5-yl)picolinamido)phenyl)-1,2,4-oxadiazol-5-yl)-1-methylpiperidin-3-yl)carbamate (12)

To a solution of benzyl ((3S,5R)-5-(3-(4-(6-bromopicolinamido)-3-fluorophenyl)-1,2,4-oxadiazol-5-yl)-1-methylpiperidin-3-yl)carbamate(200 mg, 0.36 mmol) in 1,4-dioxane/2N Na₂CO₃ (10 mL/2 mL) was added 1-(tetrahydro-2H-pyran-2-yl)-5-(4,4,5,5-tetramethyl-1,3,2-dioxaborolan-2-yl)-1H-pyrazole (131 mg, 0.47 mmol), Pd(dppf)Cl₂ (26.5 mg, 0.036 mmol), t-BuXPhos (30.8 mg, 0.072 mmol). The resulting mixture was stirred at 90°C under nitrogen atmosphere overnight and concentrated. The residue was purified by flash chromatography (eluting with dichloromathane: 10% methanol in dichloromethane=0-23%) to give compound 12 (115 mg, yellow oil, yield 89.4%). LCMS (m/z): 598.0 [M+H−THP]⁺.

### Synthesis of N-(4-(5-((3R,5S)-5-amino-1-methylpiperidin-3-yl)-1,2,4-oxadiazol-3-yl)-2-fluorophenyl)-6-(1H-pyrazol-5-yl)picolinamide (13)

To a solution of benzyl ((3S,5R)-5-(3-(3-fluoro-4-(6-(1-(tetrahydro-2H-pyran-2-yl)-1H-pyrazol-5-yl)picolinamido)phenyl)-1,2,4-oxadiazol-5-yl)-1-methylpiperidin-3-yl)carbamate (110 mg, 0.162 mmol) in THE (6 mL) was added HBr (3.5 mL, 33% in AcOH), and the resulting mixture was stirred at room temperature overnight. The reaction solution was concentrated, and the residue was purified by reverse phase chromatography (10 mmol NH₄HCO₃: CH₃CN=0-60%) to give compound 13 (50 mg, yellow oil, yield 66.9%). LCMS (m/z): 463.0 [M+H−THP]⁺.

### Synthesis of N-(4-(5-((3R,5S)-5-acrylamido-1-methylpiperidin-3-yl)-1,2,4-oxadiazol-3-yl)-2-fluorophenyl)-6-(1H-pyrazol-5-yl)picolinamide (Compound 094)

To a solution of N-(4-(5-((3R,5S)-5-amino-1-methylpiperidin-3-yl)-1,2,4-oxadiazol-3-yl)-2-fluorophenyl)-6-(1H-pyrazol-5-yl)picolinamide (50 mg, 0.108 mmol) and aq. sat. NaHCO₃ (1 mL) in THE (3 mL) at 0°C was added dropwisely a solution of acryloyl chloride (10.8 mg, 0.119 mmol) in THE (1 mL). The resulting mixture was stirred at 0°C for 1 h, and diluted with water (10 mL), the aqueous phase was extracted by ethyl acetate (15 mL×3). The combined organic phase was concentrated, the residue was purified by Prep-HPLC to give compound 094 (4.2 mg, white solid, yield 7.5%). LCMS (m/z): 517.0 [M+H]⁺. ¹H NMR (400 MHz, DMSO-d₆) δ 10.84 (s, 1H), 8.53-8.50 (m, 1H), 8.22-8.07 (m, 4H), 7.96-7.80 (m, 4H), 7.11 (d, J=2.0 Hz, 1H), 6.24-6.13 (m, 2H), 5.70 (dd, J=9.2, 2.4 Hz, 1H), 4.26-4.20 (m, 1H), 3.96-3.74 (m, 2H), 2.93 (s, 3H), 2.86-2.79 (m, 1H), 2.49-2.45 (m, 1H), 1.86-1.84 (m, 2H), 1.25-1.22 (m, 1H).

### N-(4-(5-((3R,5S)-5-acrylamido-1-methylpiperidin-3-yl)-1,2,4-oxadiazol-3-yl)-2-fluorophenyl)-6-(4-methyl-1H-pyrazol-5-yl)picolinamide (Compound 096)

Prepared according to the procedure for 094 to give 4.5 mg of the desired product in 7.5% yield. LCMS (m/z): 531.0[M+H]⁺. ¹H NMR (400 MHz, DMSO-d₆) δ 10.54 (s, 1H), 8.73 (s, 1H), 8.18-8.08 (m, 5H), 7.95-7.50 (m, 3H), 6.24-6.13 (m, 2H), 5.61 (dd, J=10.0, 2.4 Hz, 1H), 3.96-3.90 (m, 1H), 3.46-3.40 (m, 1H), 3.26-2.90 (m, 3H), 2.53 (s, 3H), 2.46-2.39 (m, 1H), 2.28 (s, 3H), 2.13-2.06 (m, 1H), 1.80-1.70 (m, 1H), 1.58-1.50 (m, 1H).

### N-(4-(5-((3R,5S)-5-acrylamidopiperidin-3-yl)-1,2,4-oxadiazol-3-yl)-2-fluorophenyl)-6-(1H-pyrazol-5-yl)picolinamide (Compound 098)

Prepared according to the procedure for 094 to give 6 mg of the desired product in 41% yield. LCMS (m/z): 503.0 [M+H]⁺. ¹H NMR (400 MHz, DMSO-d₆) δ 13.97-13.24 (m, 1H), 10.93-10.74 (m, 1H), 8.71-7.50 (m, 8H), 7.13 (s, 1H), 6.23-6.07 (m, 2H), 5.60 (dd, J=10.0, 2.4 Hz, 1H), 3.87-3.76 (m, 1H), 3.31-3.24 (m, 2H), 3.09-3.00 (m, 1H), 2.69-2.54 (m, 2H), 2.40-2.32 (m, 1H), 2.27 (t, J=11.2 Hz, 1H), 1.72 (q, J=12.0 Hz, 1H).

### Example 2: Biological Assays

To measure the IC₅₀ values of compounds herein against IRAK4, a Z′-LYTE assay (ThermoFisher) was used. Briefly, 2.5 μL of different concentrations of the compounds in 1% DMSO were added to 2.4 μL kinase buffer (50 mM HEPES pH 7.5, 0.01% BRIJ-35, 10 mM MgCl₂, 1 mM EGTA) in each well of a 384-well plate (Corning Cat. #3676). 5 μL of 2× IRAK4/Ser/Thr 07 mixture (prepared in 50 mM HEPES pH 7.5, 0.01% BRIJ-35, 10 mM MnCl₂, 2 mM DTT, and 0.02% NaN₃) and 2.5 μL of 4× ATP solution (4× ATP, 50 mM HEPES, pH 7.5, 0.01% BRIJ-35, 10 mM MgCl₂, 1 mM EGTA) were added to each well. The plate was shaken for 30 seconds, and then incubated at room temperature for 60 minutes. 5 μL of a 1:100000 dilution of Development Reagent A was added to each well. The plate was shaken for 30 seconds and incubated for 60 minutes at room temperature. The plate was subsequently read on a fluorescence plate reader, and the emissions ratio was calculated to determine the ratio of Ser/Thr 07 phosphorylated by the reaction. Emissions Ratio=Coumarin Emission (443 nm)/Flourescein Emission (520 nm).

To measure the IC₅₀ values of the compounds herein against IRAK1, the Adapta Universal Kinase Assay (ThermoFisher) was used. Briefly, 100 nL of different concentrations of the compounds in 100% DMSO were added to each well of a 384-well plate (Corning Cat. #4512). 2.4 μL of 30 mM HEPES, 2.5 μL of 4× ATP solution (in water), and 5 μL of 2× IRAK1/Histone H3 (1-20) peptide mixture (prepared in 50 mM HEPES pH 7.5, 0.01% BRIJ-35, 10 mM MgCl₂, 1 mM EGTA) were added to each well. The plate was shaken for 30 seconds and centrifuged for 1 minute at 1000×g. The plate was then incubated at room temperature for 60 minutes. 5 μL of Detection Mix was added to each well. The plate was shaken for 30 seconds and centrifuged for 1 minute at 1000×g. The plate was then incubated at room temperature for 60 minutes. The plate was subsequently read on a fluorescence plate reader, and the emissions ratio was calculated to determine the ratio of ATP to ADP. Emissions Ratio=AF647 Emission (665 nm)/Europium Emission (615 nm).

The data obtained from these assays are shown in Table A below.

TABLE A

| Compound No. | IRAK1 IC₅₀ (nM) | IRAK4 IC₅₀ (nM) |
|---|---|---|
| 001 | 19.8 | >10,000 |
| 002 | 6.42 | >10,000 |
| 005 | 96.1 | 9340 |
| 006 | 8.21 | >10,000 |
| 007 | 3.98 | >10,000 |
| 008 | 5.11 | >10,000 |
| 009 | 3.98 | >10,000 |
| 010 | 5.11 | >10,000 |
| 017 | >10,000 | >10,000 |
| 018 | 3.95 | 125 |
| 019 | 5.0 | 331 |
| 020 | 2.6 | 333 |
| 022 | 5.35 | 376 |
| 023 | 6.07 | 244 |
| 024 | 6.54 | 474 |
| 025 | 2.22 | 353 |
| 026 | 6.86 | 365 |
| 061 | 84.3 | >10,000 |
| 062 | 94.2 | >10,000 |
| 063 | 14.8 | >10,000 |
| 064 | 30.4 | >10,000 |
| 068 | 324 | 5760 |
| 070 | 1200 | 3330 |
| 071 | 609 | 8220 |
| 075 | 36.6 | >10,000 |
| 076 | 16.4 | >10,000 |
| 077 | 284 | >10,000 |
| 078 | 521 | >10,000 |
| 079 | 4.46 | 978 |
| 080 | 8.25 | 3620 |
| 081 | 9.0 | 794 |
| 082 | 17.6 | >10,000 |
| 083 | 209 | >10,000 |
| 084 | 1.67 | 204 |
| 085 | 6.82 | 8750 |
| 086 | 9.16 | >10,000 |
| 087 | 5720 | >10,000 |
| 088 | 12.7 | >10,000 |
| 089 | 21.1 | >10,000 |
| 090 | 6.62 | >10,000 |
| 091 | 52.5 | 4970 |
| 092 | 9.93 | >10,000 |
| 093 | 8.3 | >10,000 |
| 098 | 45.9 | >10,000 |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1.-61. (canceled)

62. A compound of Formula: 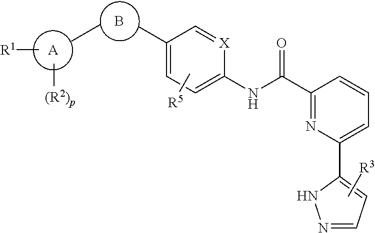
or a pharmaceutically acceptable salt thereof;
wherein
A is 3-8 membered heterocycloalkyl;
B is oxadiazolyl;
X is CH or N;
R² is independently, at each occurrence, selected from the group consisting of halogen, C₁-C₆ alkyl, C₁-C₆ haloalkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, 5-10 membered heteroaryl, OR⁴, N(R⁴)₂, SO₂R⁴, and SR⁴;
alternatively, two R², together with the carbon atom to which they are attached, form 3-8 membered cycloalkyl or 3-8 membered heterocycloalkyl;
R³ is selected from the group consisting of hydrogen, C₁-C₆ alkyl, OH, CN, NO₂, halogen, C₁-C₆ haloalkyl, C₁-C₆ alkoxy, C₁-C₆ alkylamine, C₁-C₆ haloalkyl-C₃-C₆ cycloalkyl;
R⁴ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R⁵ is selected from the group consisting of hydrogen, halogen, C₁-C₆ alkyl, C₁-C₆ haloalkyl, C₁-C₆ haloalkoxy, and C₁-C₆ alkoxy;
R^{t} is selected from the group consisting of:

| 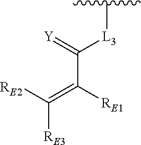 | (i-1) |
|---|---|
| 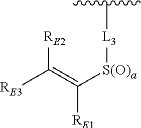 | (i-2) |
| 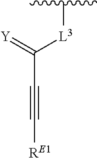 | (i-3) |
| 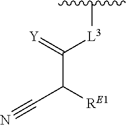 | (i-4) |
| 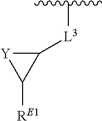 | (i-6) |
| 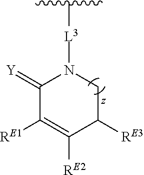 | (i-7) |
| 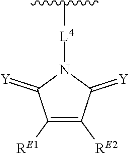 | (i-8) |
| 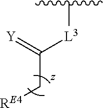 | (i-9) |
| 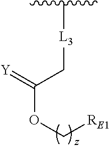 | (i-11) |
| 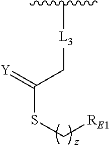 | (i-12) |
| 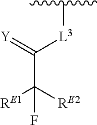 | (i-13) |
| 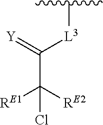 | (i-14) |
| 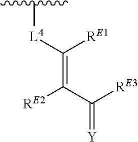 | (i-16) |
| 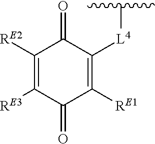 | (i-17) |
| 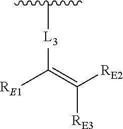 | (i-18) |
| 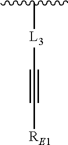 | (i-19) |
| 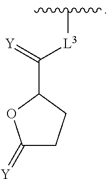 | (i-21) |
| 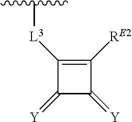 | (i-22) |
| 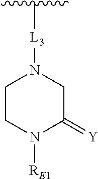 | (i-23) |
| 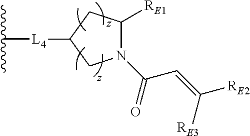 | (i-24) |
| 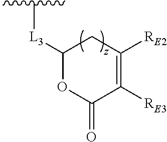 | (i-26) |
| 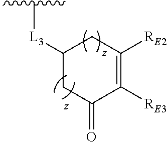 | (i-27) |
| 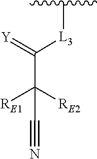 | (i-28) |
| 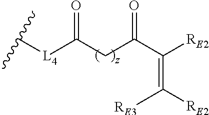 | (i-29) |
| 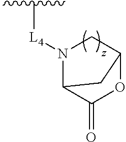 | (i-31) |
| 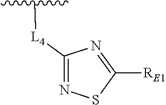 | (i-32) |
| 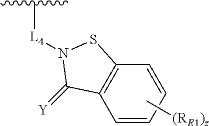 | (i-33) |
| 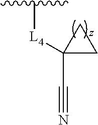 | (i-34) |
| 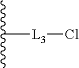 | (i-36) |
| 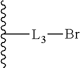 | (i-37) |
| 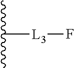 | (i-38) |
| 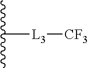 | (i-39) |
| 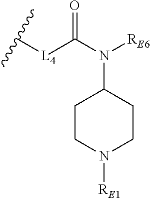 | (i-40) |
| and | |
| 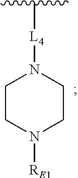 | (i-41) |

L3 is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, CiS-CR_{L3b}═CR_{L3b}—, —C≡C—, —S(O)—, —S(O)O—, —OS(O)—, —S(O)NR_{L3a}, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L4 is a bond or C₁-C₆ alkyl optionally substituted with one, two, or three R⁷;
each of R_{E1}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7};
R_{E7} is hydrogen or C₁-C₆ alkyl; and
p is 0, 1, or 2.

63. The compound of claim 62, wherein the compound is of Formula 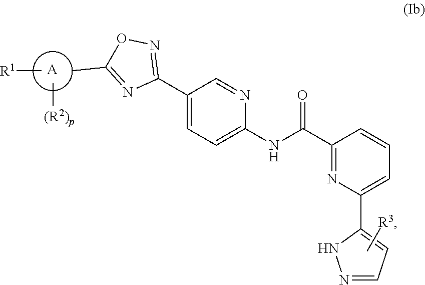
or a pharmaceutically acceptable salt thereof.

64. The compound of claim 62, wherein R^{t} is selected from the group consisting of

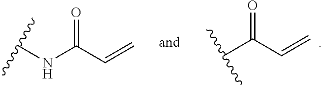

65. The compound of claim 62, wherein A is selected from the group consisting of

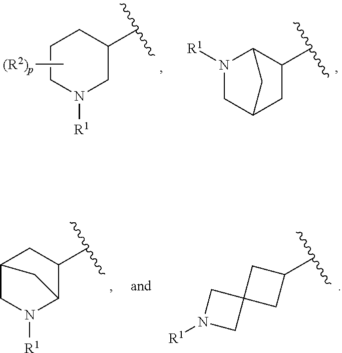

66. The compound of claim 62, wherein the compound is selected from the group consisting of: 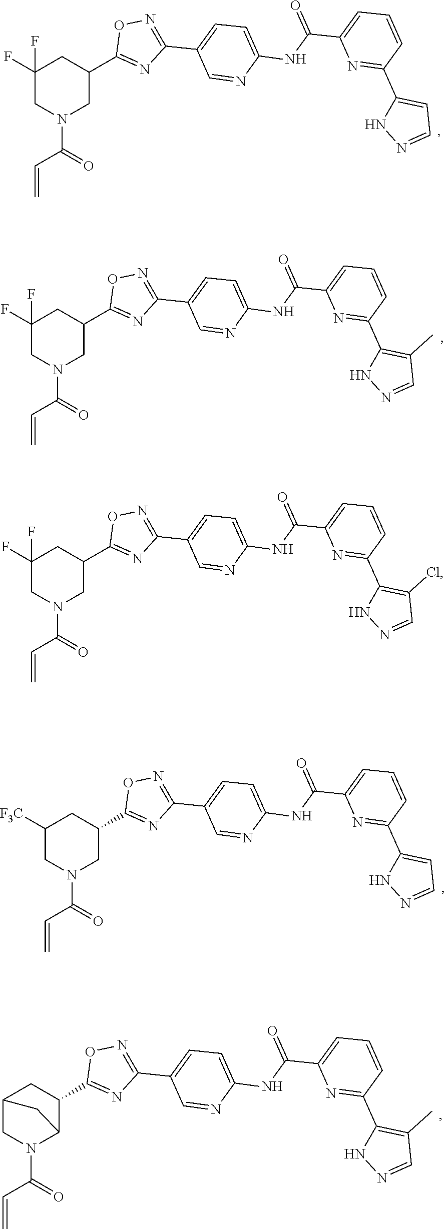 [SMILES: C=CC(=O)N1CC(c2nc(-c3ccc(NC(=O)c4cccc(-c5ccn[nH]5)n4)nc3)no2)CC(F)(F)C1 ; C=CC(=O)N1CC(c2nc(-c3ccc(NC(=O)c4cccc(-c5[nH]ncc5C)n4)nc3)no2)CC(F)(F)C1 ; C=CC(=O)N1CC(c2nc(-c3ccc(NC(=O)c4cccc(-c5[nH]ncc5Cl)n4)nc3)no2)CC(F)(F)C1 ; C=CC(=O)N1CC(C(F)(F)F)C[C@H](c2nc(-c3ccc(NC(=O)c4cccc(-c5ccn[nH]5)n4)nc3)no2)C1 ; C=CC(=O)N1CC2CC1[C@@H](c1nc(-c3ccc(NC(=O)c4cccc(-c5[nH]ncc5C)n4)nc3)no1)C2] 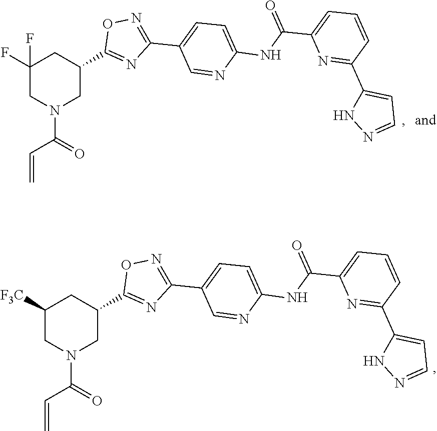 [SMILES: C=CC(=O)N1C[C@@H](c2nc(-c3ccc(NC(=O)c4cccc(-c5ccn[nH]5)n4)nc3)no2)CC(F)(F)C1 ; C=CC(=O)N1C[C@@H](c2nc(-c3ccc(NC(=O)c4cccc(-c5ccn[nH]5)n4)nc3)no2)C[C@H](C(F)(F)F)C1]
or a pharmaceutically acceptable salt thereof.

67. The compound of claim 62, wherein the compound is selected from the group consisting of: 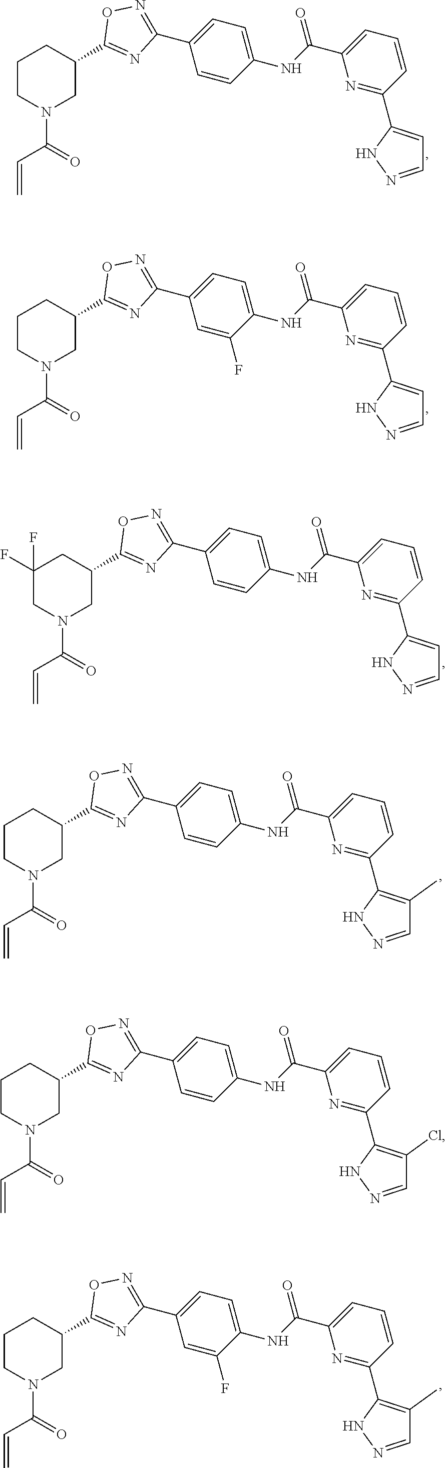 [SMILES: C=CC(=O)N1CCC[C@H](c2nc(-c3ccc(NC(=O)c4cccc(-c5ccn[nH]5)n4)cc3)no2)C1 ; C=CC(=O)N1CCC[C@H](c2nc(-c3ccc(NC(=O)c4cccc(-c5ccn[nH]5)n4)c(F)c3)no2)C1 ; C=CC(=O)N1C[C@@H](c2nc(-c3ccc(NC(=O)c4cccc(-c5ccn[nH]5)n4)cc3)no2)CC(F)(F)C1 ; C=CC(=O)N1CCC[C@H](c2nc(-c3ccc(NC(=O)c4cccc(-c5[nH]ncc5C)n4)cc3)no2)C1 ; C=CC(=O)N1CCC[C@H](c2nc(-c3ccc(NC(=O)c4cccc(-c5[nH]ncc5Cl)n4)cc3)no2)C1 ; C=CC(=O)N1CCC[C@H](c2nc(-c3ccc(NC(=O)c4cccc(-c5[nH]ncc5C)n4)c(F)c3)no2)C1] 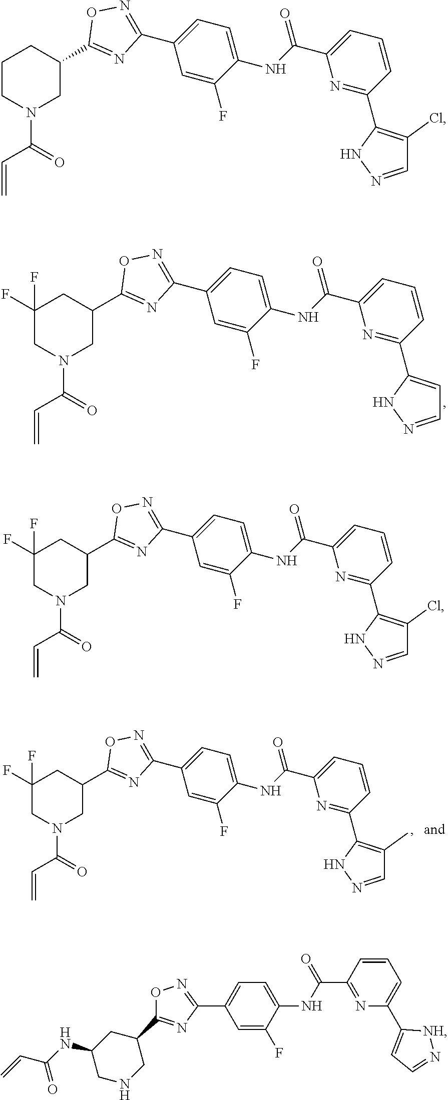
or a pharmaceutically acceptable salt thereof.

68. A pharmaceutical composition comprising a compound of claim 62, or a pharmaceutically acceptable salt thereof, and a pharmaceutically acceptable carrier.

69. A method of inhibiting interleukin-1 receptor-associated kinase 1 (IRAK1) in a subject in need thereof comprising administering to the subject a therapeutically effective amount of a compound of clam 62.

70. A method of treating a proliferative disease associated with overexpression of interleukin-1 receptor-associated kinase 1 (IRAK1) in a subject in need thereof comprising administering to the subject a therapeutically effective amount of a compound of claim 62, wherein the proliferative disease is cancer, an inflammatory disease, or an autoimmune disease.

71. The method of claim 70, wherein the proliferative disease is cancer, including breast cancer, Waldenstrom macroglobulinemia, myelodysplastic syndrome (MDS), leukemia, and lymphoma.

72. The method of claim 71, wherein the cancer is triple-negative breast cancer (TNBC).

73. The method of claim 71, wherein the cancer is acute myeloid leukemia (AML).

74. The method of claim 70, wherein the method further comprises administering a second pharmaceutical agent, including a Bruton's tyrosine kinase (BTK) inhibitor.

75. A compound of Formula: 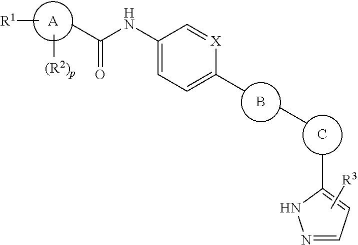
or a pharmaceutically acceptable salt thereof;
wherein
A is 3-8 membered heterocycloalkyl;
B is selected from the group consisting of —NHC(O)—, triazolyl, oxadiazolyl, oxazolyl, thiazolyl, imidazolyl, isothiazolopyridinylamino, and pyrazolyl;
C is thiazolyl or pyridinyl; provided that C is not pyridinyl when B is —NHC(O)—;
X is N or CH;
R² is independently, at each occurrence, selected from the group consisting of halogen, C₁-C₆ alkyl, C₁-C₆ haloalkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, 5-10 membered heteroaryl, OR⁴, N(R⁴)₂, SO₂R⁴, and SR⁴;
alternatively, two R², together with the carbon atom to which they are attached, form 3-8 membered cycloalkyl or 3-8 membered heterocycloalkyl;
R³ is selected from the group consisting of hydrogen, C₁-C₆ alkyl, OH, CN, NO₂, halogen, C₁-C₆ haloalkyl, C₁-C₆ alkoxy, and C₁-C₆ alkylamine;
R⁴ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R¹ is selected from the group consisting of:

| 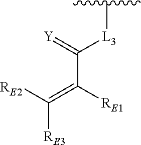 | (i-1) |
|---|---|
| 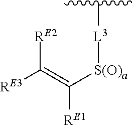 | (i-2) |
| 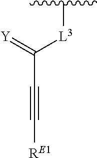 | (i-3) |
| 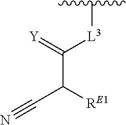 | (i-4) |
| 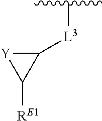 | (i-6) |
| 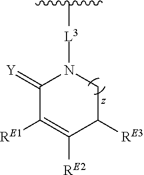 | (i-7) |
| 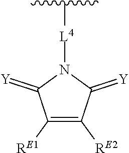 | (i-8) |
| 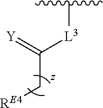 | (i-9) |
| 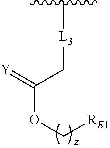 | (i-11) |
| 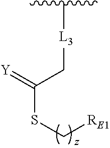 | (i-12) |
| 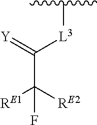 | (i-13) |
| 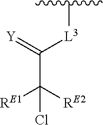 | (i-14) |
| 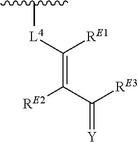 | (i-16) |
| 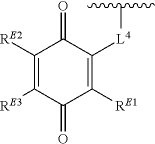 | (i-17) |
| 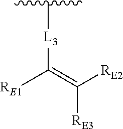 | (i-18) |
| 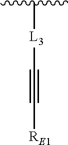 | (i-19) |
| 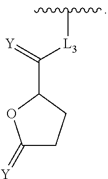 | (i-21) |
| 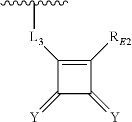 | (i-22) |
| 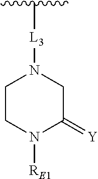 | (i-23) |
| 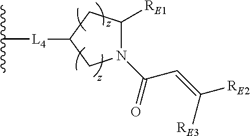 | (i-24) |
| 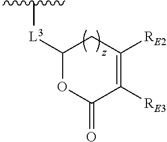 | (i-26) |
| 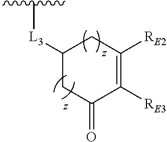 | (i-27) |
| 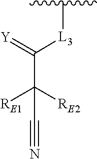 | (i-28) |
| 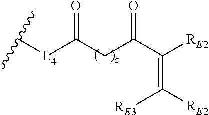 | (i-29) |
| 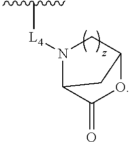 | (i-31) |
| 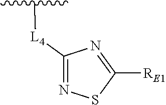 | (i-32) |
| 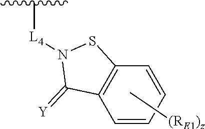 | (i-33) |
| 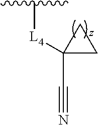 | (i-34) |
| 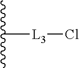 | (i-36) |
| 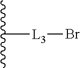 | (i-37) |
| 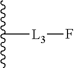 | (i-38) |
| 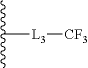 | (i-39) |
| 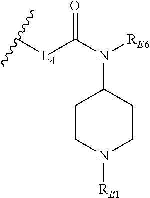 | (i-40) |
| and | |
| 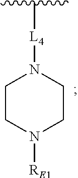 | (i-41) |

L₃ is a bond, —NH—, or C₁-C₄ alkylene, optionally wherein one or more carbon is independently replaced with —C(O)—, —O—, —S—, —NR_{L3a}, —NR_{L3a}C(O)—, —C(O)NR_{L3a}—, SC(O)——C(O)S—, —OC(O)—, —C(O)O—, —NR_{L3a}C(S)—, —C(S)NR_{L3a}—, trans-CR_{L3b}═CR_{L3b}—, CiS-CR_{L3b}═CR_{L3b}—, —C≡C—, —S(O)—, —S(O)O—, —OS(O)—, —S(O)NR_{L3a}, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E1}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7};
R_{E7} is hydrogen or C₁-C₆ alkyl; and
p is 0, 1, or 2.

76. The compound of a claim 75, wherein R^{t} is selected from the group consisting of

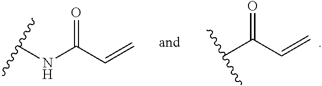

77. The compound of claim 75, wherein the compound is selected from the group consisting of: 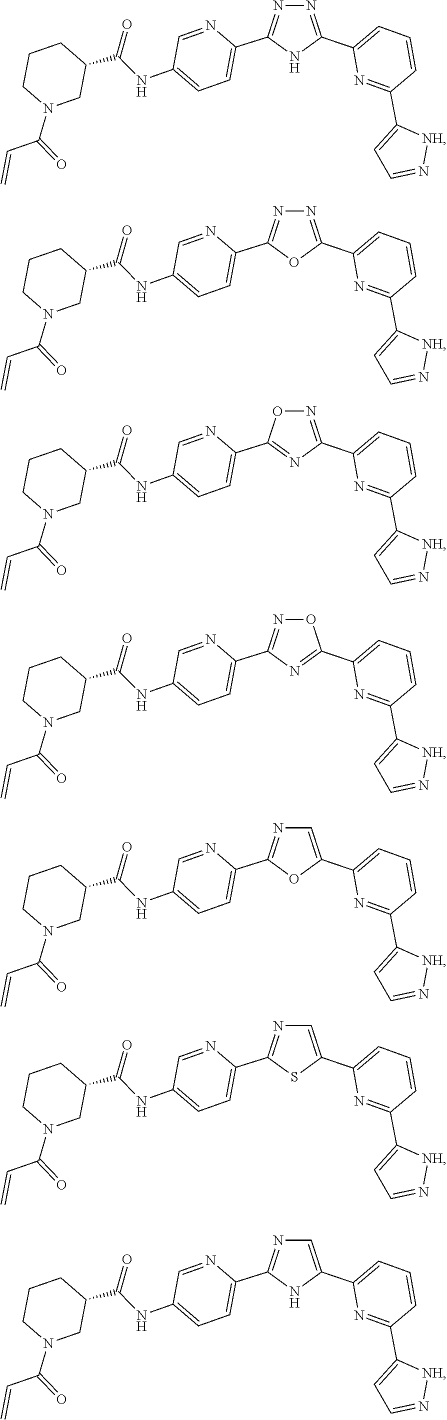 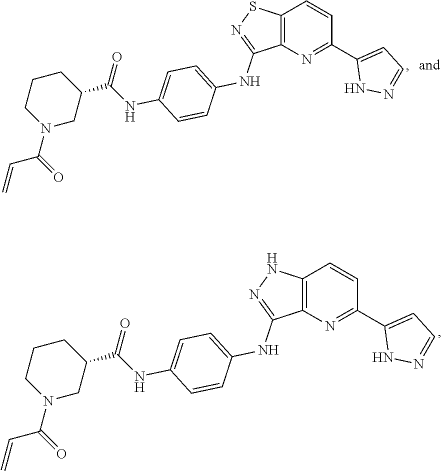 [SMILES: C=CC(=O)N1CCC[C@H](C(=O)Nc2ccc(Nc3nsc4ccc(-c5ccn[nH]5)nc34)cc2)C1 ; C=CC(=O)N1CCC[C@H](C(=O)Nc2ccc(Nc3n[nH]c4ccc(-c5ccn[nH]5)nc34)cc2)C1]
or a pharmaceutically acceptable salt thereof.

78. A pharmaceutical composition comprising a compound of claim 75, or a pharmaceutically acceptable salt thereof, and a pharmaceutically acceptable carrier.

79. A method of inhibiting interleukin-1 receptor-associated kinase 1 (IRAK1) in a subject in need thereof comprising administering to the subject a therapeutically effective amount of a compound of claim 75.

80. A method of treating a proliferative disease associated with overexpression of interleukin-1 receptor-associated kinase 1 (IRAK1) in a subject in need thereof comprising administering to the subject a therapeutically effective amount of a compound of claim 75, wherein the proliferative disorder is cancer, an inflammatory disease, or an autoimmune disease.

81. The method of claim 79, wherein the method further comprises administering a second pharmaceutical agent, including a Bruton's tyrosine kinase (BTK) inhibitor.

82. A compound of Formula: 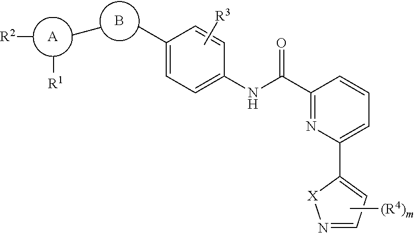
or a pharmaceutically acceptable salt thereof;
wherein
X is NH or O;
A is 6-8 membered heterocycloalkyl or C₅ cycloalkyl;
B is triazolyl;
R² is H or C₁-C₆ alkyl;
R³ is H or C₁-C₆ alkoxy;
R⁴ is halo;
m is 0 or 1;
R¹ is selected from the group consisting of:

| 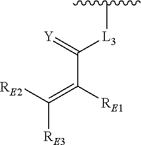 | (i-1) |
|---|---|
| 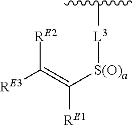 | (i-2) |
| 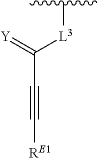 | (i-3) |
| 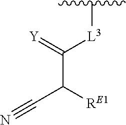 | (i-4) |
| 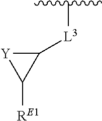 | (i-6) |
| 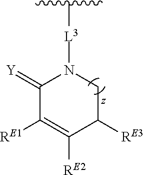 | (i-7) |
| 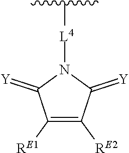 | (i-8) |
| 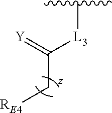 | (i-9) |
| 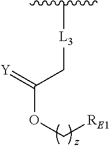 | (i-11) |
| 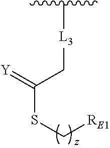 | (i-12) |
| 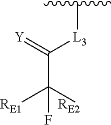 | (i-13) |
| 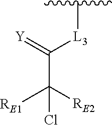 | (i-14) |
| 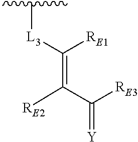 | (i-16) |
| 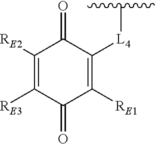 | (i-17) |
| 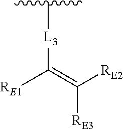 | (i-18) |
| 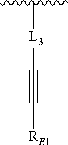 | (i-19) |
| 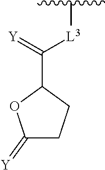 | (i-21) |
| 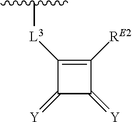 | (i-22) |
| 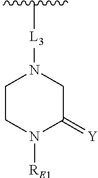 | (i-23) |
| 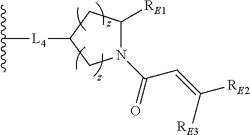 | (i-24) |
| 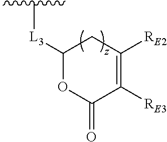 | (i-26) |
| 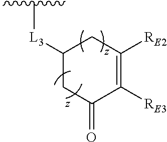 | (i-27) |
| 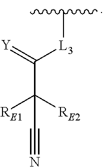 | (i-28) |
| 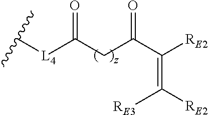 | (i-29) |
| 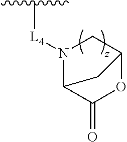 | (i-31) |
| 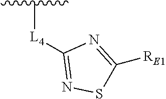 | (i-32) |
| 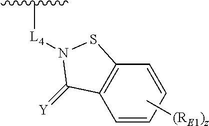 | (i-33) |
| 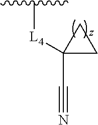 | (i-34) |
| 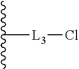 | (i-36) |
| 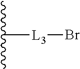 | (i-37) |
| 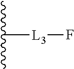 | (i-38) |
| 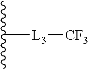 | (i-39) |
| 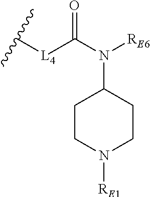 | (i-40) |
| and | |
| 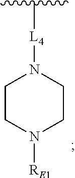 | (i-41) |

L₃ is a bond, —NH—, or C₁-C₄ alkylene, optionally wherein one or more carbon is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, CiS-CR_{L3b}═CR_{L3b}—, —C≡C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen or C₁-C₆ alkyl optionally substituted with R⁷;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁷;
or, alternatively, two R_{L3b} groups, together with the atoms to which they are attached, form a 3-8 membered cycloalkyl or 4-7 membered heterocycloalkyl, both of which are optionally substituted with one, two, or three R⁷;
L₄ is a bond or C₁-C₆ alkyl optionally substituted with one, two, or three R⁷;
each of R_{E1}, R_{E2}, and R_{E3} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁷;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁷;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁷;
or, alternatively, two R_{EE} groups, together with the atom to which they are attached, form 4-7 membered heterocycloalkyl;
R⁷ is independently, at each occurrence, selected from the group consisting of hydrogen, halogen, C₁-C₆ alkyl, C(O)C₁-C₆ alkyl, C₂-C₆ alkenyl, C₂-C₆ alkynyl, 3-8 membered cycloalkyl, 3-8 membered heterocycloalkyl, 6-10 membered aryl, and 5-10 membered heteroaryl;
R_{E5} is halogen;
R_{E6} is hydrogen or C₁-C₆ alkyl;
each Y is independently O, S, or NR_{E7}; and
R_{E7} is hydrogen or C₁-C₆ alkyl.

83. The compound of a claim 82, wherein R³ is methoxy.

84. The compound of claim 82, wherein the compound is selected from the group consisting of: 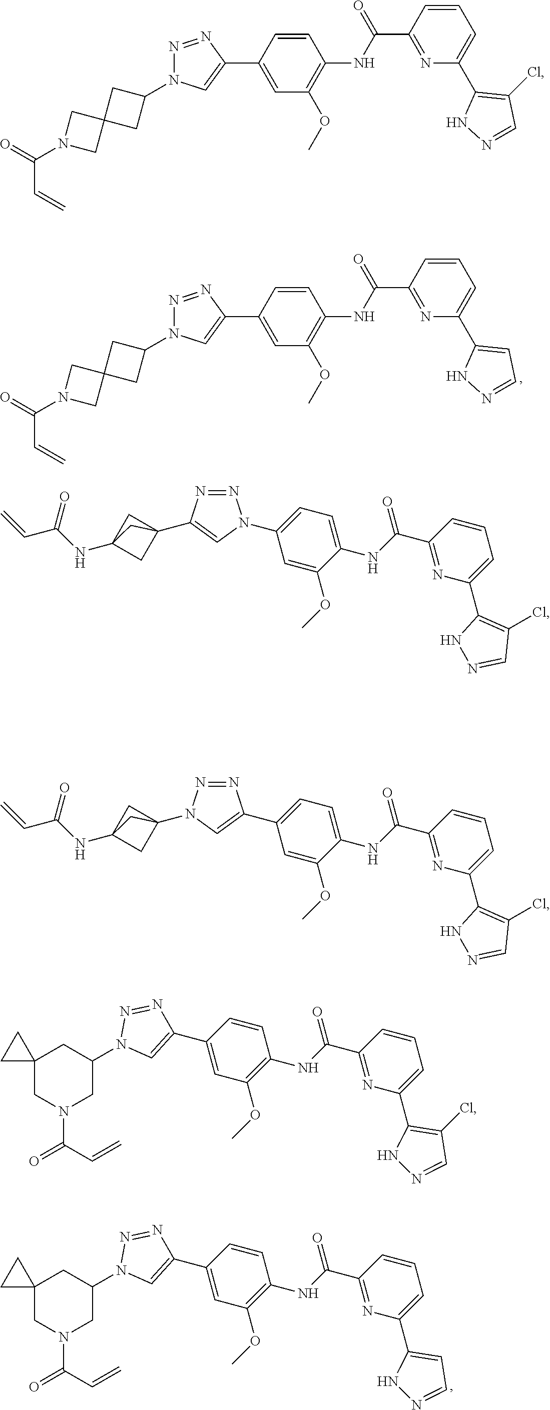 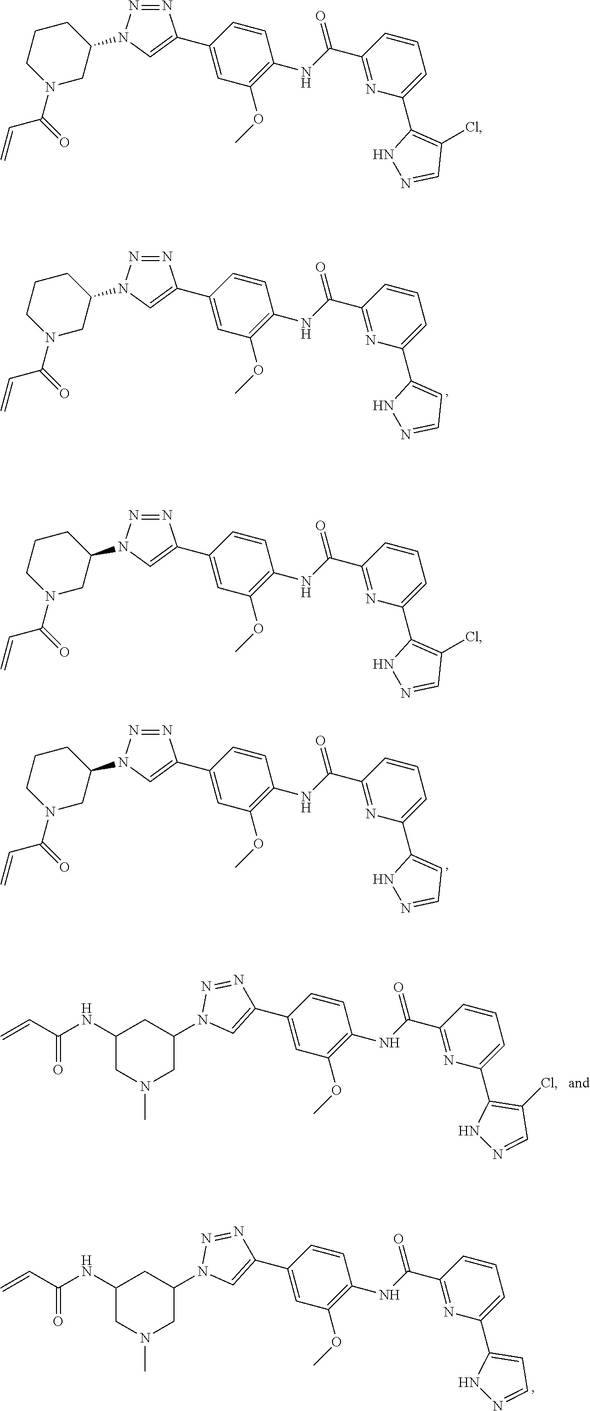
or a pharmaceutically acceptable salt thereof.

85. A compound selected from the group consisting of: 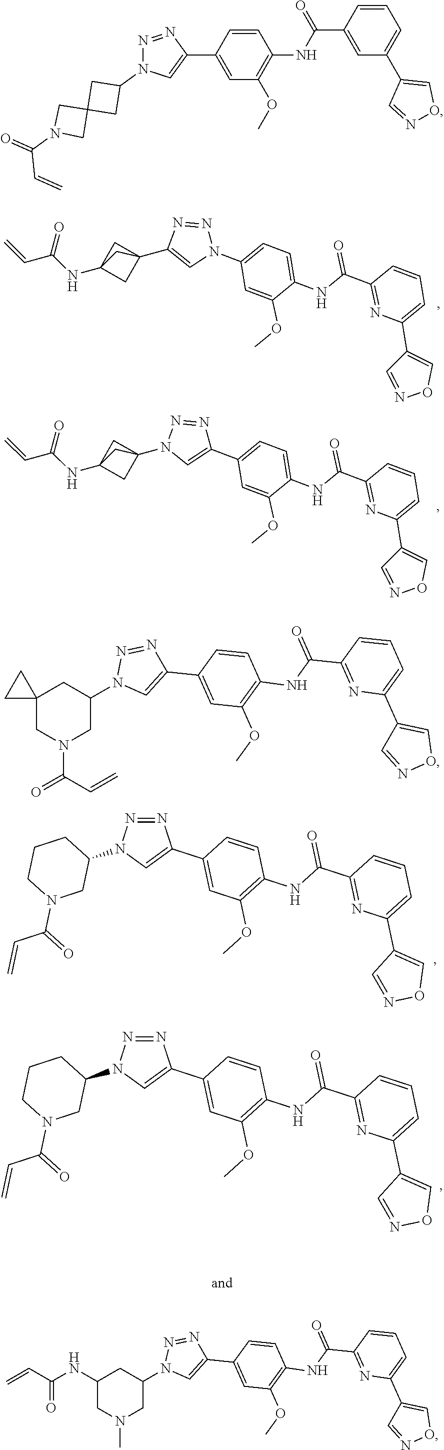
or a pharmaceutically acceptable salt thereof.
